SEQUENCE LISTING (155 sequences)

SEQ ID NO: 1 - protein, 245 aa, Artificial
  EBV BZLF1 Strain AG876
MMDPNSTSEDVKFTPDPYQVPFVQAFDQATRVYQDLGGPSQAPLPCVLWPVLPEPLPQGQLTAYHVSAAPTGSWFPAPQPAPENAYQAYAAPQLFPVSDITQNQQTNQAGGEAPQPGDNSTVQPAAAVVFACPGANQGQQLADIGAPQPAPAAAPARRTRKPLQPESLEECDSELDIKRYKNRVASRKCRAKFKHLLQHYREVASAKSSENDRLRLLLKQMCPSLDVDSIIPRTPDVLHEDLLNF

SEQ ID NO: 2 - protein, 245 aa, Artificial
  EBV BZLF1 Strain B95-8
MMDPNSTSEDVKFTPDPYQVPFVQAFDQATRVYQDLGGPSQAPLPCVLWPVLPEPLPQGQLTAYHVSTAPTGSWFSAPQPAPENAYQAYAAPQLFPVSDITQNQQTNQAGGEAPQPGDNSTVQTAAAVVFACPGANQGQQLADIGVPQPAPVAAPARRTRKPQQPESLEECDSELEIKRYKNRVASRKCRAKFKQLLQHYREVAAAKSSENDRLRLLLKQMCPSLDVDSIIPRTPDVLHEDLLNF

SEQ ID NO: 3 - protein, 245 aa, Artificial
  EBV BZLF1 Strain GD1
MMDPNSTSEDVKFTPDPYQVPFVQAFDQATRVYQDLGGPSQAPLPCVLWPVLPEPLPQGQLTAYHVSAAPTGSWFPAPQPAPENAYQAYAAPQLFPVSDITQNQLTNQAGGEAPQPGDNSTVQPAAAVVLACPGANQEQQLADIGAPQPAPAAAPARRTRKPLQPESLEECDSELEIKRYKNRVASRKCRAKFKHLLQHYREVASAKSSENDRLRLLLKQMCPSLDVDSIIPRTPDVLHEDLLNF

SEQ ID NO: 4 - protein, 641 aa, Artificial
  EBV EBNA1 Strain AG876
MSDEGPGTGPGNGLGQKEDTSGPDGSSGSGPQRRGGDNHGRGRGRGRGRGGGRPGAPGGSGSGPRHRDGVRRPQKRPSCIGCKGAHGGTGAGGGAGAGGAGAGGAGAGGAGAGGAGAGGAGAGGAGAGGAGAGGAGAGGAGAGGGAGAGGAGAGGAGAGGGAGAGGGAGAGGGAGAGGGAGAGGGAGAGGGAGAGGGAGAGGGAGAGGGAGAGGAGAGGAGAGGGAGAGGGAGAGGGAGAGGGAGAGGGAGAGGGAGAGGGAGAGGGAGAGGGAGAGGGAGAGGGAGAGGGAGAGGGAGAGGGAGAGGGAGAGGGAGAGGGAGAGGGGRGRGGSGGRGRGGSGGRGRGGSGGRRGRGRERARGGSRERARGRGRGRGEKRPRSPSSQSSSSGSPPRRPPPGRRPFFHPVAEADYFEYHQEGGPDGEPDMPPGAIEQGPADDPGEGPSTGPRGQGDGGRRKKGGWYGKHRGEGGSSQKFENIAEGLRLLLARCHVERTTEDGNWVAGVFVYGGSKTSLYNLRRGIGLAIPQCRLTPLSRLPFGMAPGPGPQPGPLRESIVCYFIVFLQTHIFAEGLKDAIKDLVLPKPAPTCNIKVTVCSFDDGVDLPPWFPPMVEGAAAEGDDGDDGDEGGDGDEGEEGQE

SEQ ID NO: 5 - protein, 641 aa, Artificial
  EBV EBNA1 Strain B95-8
MSDEGPGTGPGNGLGEKGDTSGPEGSGGSGPQRRGGDNHGRGRGRGRGRGGGRPGAPGGSGSGPRHRDGVRRPQKRPSCIGCKGTHGGTGAGAGAGGAGAGGAGAGGGAGAGGGAGGAGGAGGAGAGGGAGAGGGAGGAGGAGAGGGAGAGGGAGGAGAGGGAGGAGGAGAGGGAGAGGGAGGAGAGGGAGGAGGAGAGGGAGAGGAGGAGGAGAGGAGAGGGAGGAGGAGAGGAGAGGAGAGGAGAGGAGGAGAGGAGGAGAGGAGGAGAGGGAGGAGAGGGAGGAGAGGAGGAGAGGAGGAGAGGAGGAGAGGGAGAGGAGAGGGGRGRGGSGGRGRGGSGGRGRGGSGGRRGRGRERARGGSRERARGRGRGRGEKRPRSPSSQSSSSGSPPRRPPPGRRPFFHPVGEADYFEYHQEGGPDGEPDVPPGAIEQGPADDPGEGPSTGPRGQGDGGRRKKGGWFGKHRGQGGSNPKFENIAEGLRALLARSHVERTTDEGTWVAGVFVYGGSKTSLYNLRRGTALAIPQCRLTPLSRLPFGMAPGPGPQPGPLRESIVCYFMVFLQTHIFAEVLKDAIKDLVMTKPAPTCNIRVTVCSFDDGVDLPPWFPPMVEGAAAEGDDGDDGDEGGDGDEGEEGQE

SEQ ID NO: 6 - protein, 641 aa, Artificial
  EBV EBNA1 Strain GD1
MSDEGPGTGPGNGLGQKEDSSGPEGSGGSGPQRRGGDNHGRGRGRGRGRGGGRPGAPGGSGSGPRHRDGVRRPQKRPSCIGCKGAHGGTGSGAGAGGAGAGGAGAGGGAGAGGGAGGAGGAGGAGAGGGAGAGGGAGGAGGAGAGGGAGAGGGAGGAGAGGGAGGAGGAGAGGGAGAGGGAGGAGAGGGAGGAGGAGAGGGAGAGGAGGAGGAGAGGAGAGGGAGGAGGAGAGGAGAGGAGAGGAGAGGAGGAGAGGAGGAGAGGAGGAGAGEEAGGAGAGGGAGGAGAGGAGGAGAGGAGGAGAGGAGGAGAGGGAGAGGAGAGGGGRGRGGSGGRGRGGSGGRGRGGSGGRRGRGRERARGRSRERARGRGRGRGEKRPRSPSSQSSSSGSPPRRPPPGRRPFFHPVGDADYFEYLQEGGPDGEPDVPPGAIEQGPTDDPGEGPSTGPRGQGDGGRRKKGGWFGKHRGQGGSNPKFENIAEGLRVLLARSHVERTTEEGNWVAGVFVYGGSKTSLYNLRRGIALAVPQCRITPLSRLPFGMAPGPGPQPGPLRESIVCYFMVFLQTHIFAEVLKDAIKDLVMTKPAPTCNIKVTVCSFDDGVDLPPWFPPMVEGAAAEGDDGDDGDEGGDGDEGEEGQE

SEQ ID NO: 7 - protein, 925 aa, Artificial
  EBNA3 Strain AG876
MDKDRPGLPAPDDNIEEVPSTSGVQERASEGDWENVLIEISDSSSEEEAEDAHLEPSQRGKKRKRVDDDAGGSAPAQHVPPPQLDHPGREAILYRFPLDLRRFIQAIGAAATHPDTRAIDQFFGSQISNTDLYVMYAMAIRQAIRDRRRNPASRRSQVKWRMTTLAAGWPMGYQAYSSWMYSYTDPQVTATIIHLQATLGCASGRRCHVTFSAGTFRPPRCSPGDRQWLYVQSRVGDLVQSSNPCYSIFFDYMAIHRSLTKIWDEVVTPDQRVTFMEFLGFLQRTELVYIKSFVSYALGTTSIETPWMDENPSTETAQAWNAGLLRGRAYGQDLLRTEGEHGEGATCETREESEDTESDGDDEELPRVVSRDGTKHRRPPIFLRRLHRLLLMRAGKGKERARETLAKAPRRTYGTPRPPVQKPRPEVPQSYETATSHGSAQVPEPPPTHPLHQQHSMAPCMVAQNPRAPLGDQLPGVPKDGRGACAPVPALAGPIVRPWESSLLQSPGRAFAPVSPQPMPVEPVPVPTVALERPVCPAPPEIAMQGPGEPSGIKRTRERWRPAPWTPNPPRSPSQMSVRDRLARLRAEAQARQASVEVQPTQLTQVSPQQPMERPLEPEQQMFPGSPFSQVADVARESGVPAMQPQYFDLPLTQPISQGAPAAPLRASMGPVPPVPATQPQYFDIPLTEPINQGASAAHFLPQQPMEGPLVPERWMFQGATLSQSVRPGVAQSQYFDLPLTQPINHGAPAAHFLHQPPMEGPWVPEQWMFQGAPPSQGTDVVQHQLDDLGYPLHDLNHPGVPVSPAVNQYHFSQAAFGLPIDEDESGERSDTSEPYEALDLSIHGRPCPQAPEWPVQGEGGQDATEVLDLSIHGRPRPRTPEWPVQGESGQNVTDHEPRRVVVSAIVHMCQDDEFPDLQDPPDEA

SEQ ID NO: 8 - protein, 944 aa, Artificial
  EBV EBNA3 Strain B95-8
MDKDRPGPPALDDNMEEEVPSTSVVQEQVSAGDWENVLIELSDSSSEKEAEDAHLEPAQKGTKRKRVDHDAGGSAPARPMLPPQPDLPGREAILRRFPLDLRTLLQAIGAAATRIDTRAIDQFFGSQISNTEMYIMYAMAIRQAIRDRRRNPASRRDQAKWRLQTLAAGWPMGYQAYSSWMYSYTDHQTTPTFVHLQATLGCTGGRRCHVTFSAGTFKLPRCTPGDRQWLYVQSSVGNIVQSCNPRYSIFFDYMAIHRSLTKIWEEVLTPDQRVSFMEFLGFLQRTDLSYIKSFVSDALGTTSIQTPWIDDNPSTETAQAWNAGFLRGRAYGIDLLRTEGEHVEGATGETREESEDTESDGDDEDLPCIVSRGGPKVKRPPIFIRRLHRLLLMRAGKRTEQGKEVLEKARGSTYGTPRPPVPKPRPEVPQSDETATSHGSAQVPEPPTIHLAAQGMAYPLHEQHGMAPCPVAQAPPTPLPPVSPGDQLPGVFSDGRVACAPVPAPAGPIVRPWEPSLTQAAGQAFAPVRPQHMPVEPVPVPTVALERPVYPKPVRPAPPKIAMQGPGETSGIRRARERWRPAPWTPNPPRSPSQMSVRDRLARLRAEAQVKQASVEVQPPQLTQVSPQQPMEGPLVPEQQMFPGAPFSQVADVVRAPGVPAMQPQYFDLPLIQPISQGAPVAPLRASMGPVPPVPATQPQYFDIPLTEPINQGASAAHFLPQQPMEGPLVPEQWMFPGAALSQSVRPGVAQSQYFDLPLTQPINHGAPAAHFLHQPPMEGPWVPEQWMFQGAPPSQGTDVVQHQLDALGYTLHGLNHPGVPVSPAVNQYHLSQAAFGLPIDEDESGEGSDTSEPCEALDLSIHGRPCPQAPEWPVQEEGGQDATEVLDLSIHGRPRPRTPEWPVQGEGGQNVTGPETRRVVVSAVVHMCQDDEFPDLQDPPDEA

SEQ ID NO: 9 - protein, 935 aa, Artificial
  EBV EBNA3 Strain GD1
MDKDRPGDPALDDNMEEEVPSTSVVQEQVSAGDWENVLIELSDSSSEKEAEDAQLEPAQKGTKRKRVDHDAGGSAPARPMLPPQPDLPGREAILRRFPLDLRTLLQAIGAAATRIDTRAIDQFFGSQISNTEMYIMYAMAIRQAIRDRRRNPASRRDQAKWRLQTLAAGWPMGYQAYSSWMYSYTDHQTAPTFVQLQATLGCTGGRRCHVTFSAGTFKPPRCTPGDRQWLYVQSSVGNIVQSCNPRYSIFFDYMAIHRSLTKIWEEVLTPDQRVSFMEFLGFLQRTDLSYIKSFVSDALGTTSIQTPWIDDNSSTETAQAWNAGFLRGRAYGLDLLRTEGEHVEGATGETREESEDTESDGDDEDLPCIVSRGGPKVKRPPIFIRRLHRLLLMRAGKRTEQGKEVLEKARGSTYGTPRPPVPKPRPEVPQSDETATSHGSAQVPEPPTIHLAAQGMAYPLHEQRGMAPCPVAQAPHTPLPPVSPGDQLPGVSSDGRVACAPVPAPAGPIVRPWEPSLTQAAGQAFAPVRPQHMPVEPVPVPTVALERPVYPKPVRPAPPKIAMQGPGETSGIRRARERWRPAPWTPNPPRSPSQMSVRDRLARLRAEAQVKQASVEVQPPQVTQVSPQQPMEGPGAPFSQVADVVHTPGVPAMQPQYFDLPLIQPISQGAPVAPLRASMGPVPPVPATQPQYFDIPLTEPINQGASAAHFLPQQPMEGPLVPEQWMFPGAALSQSVRPGVAQSQYFDLPLTQPITHGAPAAHFLHQPPMEGPWVPEQWMFQGAPPSQGTDVVQHQLDALGYPLHALNHPGVPVSPAVNQYHLSQAAFGLPIDEDESGEGSDTSEPCEALDLSIHGRPCPQAPEWPVQGEGGQDATEVLDLSIHGRPRPRTPEWPVQGESGQNVTDHEPRRVVVSAIVHMCQDDEFPDLQDPPDEA

SEQ ID NO: 10 - protein, 946 aa, Artificial [sequence omitted]

SEQ ID NO: 11 - protein, 938 aa, Artificial
  EBV EBNA4 Strain B95-8
MKKAWLSRAQQADAGGASGSEDPPDYGDQGNVTQVGSEPISPEIGPFELSAASEDDPQSGPVEENLDAAAREEEEPHEQEHNGGDDPLDVHTRQPRFVDVNPTQAPVIQLVHAVYDSMLQSDLRPLGSLFLEQNLNIEEFIWMCMTVRHRCQAIRKKPLPIVKQRRWKLLSSCRSWRMGYRTHNLKVNSFESGGDNVHPVLVTATLGCDEGTRHATTYSAGIVQIPRISDQNQKIETAFLMARRARSLSAERYTLFFDLVSSGNTLYAIWIGLGTKNRVSFIEFVGWLCKKDHTHIREWFRQCTGRPKAAKPWLRAHPVAIPYDDPLTNEEIDLAYARGQAMNIEAPRLPDDPIIVEDDDESEEIEAESDEEEDKSGMESLKNIPQTLPYNPTVYGRPAVFDRKSDAKSTKKCRAIVTDFSVIKAIEEEHRKKKAARTEQPRATPESQAPTVVLQRPPTQQEPGPVGPLSVQARLEPWQPLPGPQVTAVLLHEESMQGVQVHGSMLDLLEKDDEVMEQRVMATLLPPVPQQPRAGRRGPCVFTGDLGIESDEPASTEPVHDQLLPAPGPDPLEIQPLTSPTTSQLSSSAPSCAQTPWPVVQPSQTPDDPTKQSRPPETAAPRQWPMPLRPIPMRPLRMQPIPFNHPVGPTPHQTPQVEITPYKPTWAQIGHIPYQPTPTGPATMLLRQWAPATMQTPPRAPTPMSPPEVPPVPRQRPRGAPTPTPPPQVPPVPRQRPRGAPTPTPPPQVLPTPMQLALRAPAGQQGPTKQILRQLLTGGVKKGRPSLKLQAALERQAAAGWQPSPGSGTSDKIVQAPIFYPPVLQPIQVMGQGGSPTAMAASAVTQAPTEYTRERRGVGPMPPTDIPPSKRAKIEAYTEPEMPHGGASHSPVVILENVGQGQQQTLECGGTAKQERDMLGLGDIAVSSPSSSETSNDE

SEQ ID NO: 12 - protein, 938 aa, Artificial
  EBV EBNA4 Strain GD1
MKKAWLSRARQADAGGASGSEDPPDYGDQGNVQQVGSGPISPEIGPFELSAASEDDPQSGPVEENLDAAAREEQEPHEQEHNGGDDPLDVHTRQPRFVDVNPTQAPVIQLVHAVYDSMLQSDLRPLGSLFLEQNLNIEEFIWMCMTVRHRCQAIRKKPLPIVKQRRWKLLSSCRSWRMGYRTHNLKVNSFESGGDNVHPVLVTATLGCDEGTRHATTYSAGIVQIPRISDQNQKIETAFLMARRARSLSAERYTLFFDLVSSGNTLYAIWIGLGTKNRVSFIEFVGWLCKKDHTHIREWFRQCTGRPKAAKPWLRAHPVAIPYDDPLTNEEIDLAYARGQAMNIEAPRLPDDPIIVEDDDESEEIEAESDEEEDKSGMESLKNIPQTLPYNPTVYGRPAVFDRKSDAKSTKKCRAILTDFSVIKAIEEEHRKKKAARTEQPRATPESQAPTVVLQRPPTQHEPGPAGPLSVQARLEPWQPLPGPQVTTVLLHEESMQGVQVHGSMLDLLEKDDEVMEQRVMATLLPPVPQQPQAGRRGPCVYTGDLGIESDELASTEPVHDQLLPAPGPDPLEIQPLTSPTTSQLSSSAPSCAQTPWPVVQPSQTPDDPMKQSRPPETAAPRQWPMPLRPIPMRPLRMQPIPFNHPVGPTPHQTPQVELTPYKPTWAQMGHIPYQPTPTGPATMLLRQWAPATMQTPPRAPTPMSPPEVPPVPRQRSRGAPTPTPPPQVPPVPRQRPRGAPTPTPPPQVLPTPMQLAPRAPAGQQGPTKQILRQLLTGGVKKGRPSLKLQAALERQAAAGWQPSTGSGTSAKIVRAPVFYPPVLQPIQVMGQGGSPTAMAASTVTQEPTEYTRERRGVGPMPPTDIPPSKRAKIEAYTEPEMPHGGASHSPVVILENVGQGQQQTLECGGTAKQERDVLGLGDIAVSSPSSSETSNDE

SEQ ID NO: 13 - protein, 1069 aa, Artificial
  EBV EBNA6 Strain AG876
MESFEGEGDSIQSPDNARGDDVQNTGEHIQDPGPGPSTGGASEGLVQNEPDSRDQQSRGQRRGDENRGWMQRIRRRRRRRAALSGHLLDMEDNVPPWFPPHDITPYVARNIRDAACQAVKHSHLQALSNLILDSGLDTQHLLCFVMAARQRLQDIRRGPLVVEGGVGWRHWLLTSPSRSWSMGYRTATLRTLTPVPNRVGADSIMLTATFGCQNGALAINTFSATVWIPPPAGPREQERYAREAEVRFLRGKWQRRFRRIFDLIELCGSLHHVWQNMLQTEENLLDFVRFMGVMSSCNSSSVNYWFHKTIGNFKPYYPWNAPPNENPYHARRGIKEQVIQKAFLKAQRQGLSMLATGGGPRGDATSETSSDEDTGRQGSDVELESSDDELPYIDPNMEPVQQRPVMFVSRVPVRKPRTLPWPTPKTHPVKRTIVKTSYRSDEAEEAQSTPERPGPSKQPSEPVEPAHTTPAGRSTVILHEPPREPEAVSFKPPPPPSRRRRGACVVYDDDIIEVIDVETTEEETTSMQRQPPLGQQPPPPVISTGSAMSSSHTDPSVTQPSKPHRKPQDGFQRSGRRQKRAMPPPVSPSDAGPPSTRPRVMAPPSTGPRVMATPSTGPRDMAPPSTGPRDMAPPSTGPRDMAPPSTGPRDMAPTVVHMFTRERLLTQSTGPAPRSFWEMRAGRDAPKIQQEPSSQQQPATQSTPPCQSWVPSVYVLPAVDAGNAQPLQISHLSSMSPTQPISHEEQPRYEDPDTPLDLSLHPDTATLPPTQDLYPGREDLQATQAPYPGYEEPRPPQAPFVGDYGFVQIPSAQWEPHPSQGTYQGHIDPQLPAALDLGPEQPRFPQDPYVYSGGQLSSCPGYAGPWPSRPQHPRYRHTLALWPREPRHGHSQGPWKPWSAHLPPQWDGSAGHGQDQVSQFPHLHSETGPPRLQLSSVPQVLYPQPLVSSSAPSWSSPQPRAPIRPIPTRFPPPPMPLQDSMAVGCDSSGTACPSMPFASDYSQGAFTPLDINAPTPKSPRVEESSHGPARCSQATSEAQEILSDNSEISVFPKDAKQTDYDASTESELD

SEQ ID NO: 14 - protein, 992 aa, Artificial
  EBV EBNA6 Strain B95-8
MESFEGQGDSRQSPDNERGDNVQTTGEHDQDPGPGPPSSGASERLVPEESYSRDQQPWGQSRGDENRGWMQRIRRRRRRRAALSGHLLDTEDNVPPWLPPHDITPYTARNIRDAACRAVKQSHLQALSNLILDSGLDTQHILCFVMAARQRLQDIRRGPLVAEGGVGWRHWLLTSPSQSWPMGYRTATLRTLTPVPNRVGADSIMLTATFGCQNAARTLNTFSATVWTPPHAGPREQERYAREAEVRFLRGKWQRRYRRIYDLIELCGSLHHIWQNLLQTEENLLDFVRFMGVMSSCNNPAVNYWFHKTIGNFKPYYPWNAPPNENPYHARRGIKEHVIQNAFRKAQIQGLSMLATGGEPRGDATSETSSDEDTGRQGSDVELESSDDELPYIDPNMEPVQQRPVMFVSRVPAKKPRKLPWPTPKTHPVKRTNVKTSDRSDKAEAQSTPERPGPSEQSSVTVEPAHPTPVEMPMVILHQPPPVPKPVPVKPTPPPSRRRRGACVVYDDDVIEVIDVETTEDSSSVSQPNKPHRKHQDGFQRSGRRQKRAAPPTVSPSDTGPPAVGPPAAGPPAAGPPAAGPPAAGPPAAGPPAAGPRILAPLSAGPPAAGPHIVTPPSARPRIMAPPVVRMFMRERQLPQSTGRKPQCFWEMRAGREITQMQQEPSSHLQSATQPTTPRPSWAPSVCALSVMDAGKAQPIESSHLSSMSPTQPISHEEQPRYEDPDAPLDLSLHPDVAAQPAPQAPYQGYQEPPAPQAPYQGYQEPPPPQAPYQGYQEPPAHGLQSSSYPGYAGPWTPRSQHPCYRHPWAPWSQDPVHGHTQGPWDPRAPHLPPQWDGSAGHGQDQVSQFPHLQSETGPPRLQLSLVPLVSSSAPSWSSPQPRAPIRPIPTRFPPPPMPLQDSMAVGCDSSGTACPSMPFASDYSQGAFTPLDINATTPKRPRVEESSHGPARCSQATAEAQEILSDNSEISVFPKDAKQTDYDASTESELD

SEQ ID NO: 15 - protein, 1009 aa, Artificial
  EBV EBNA6 Strain GD1
MESFEGQGDSRQSPDNERGDNVQTTGEHDQDPGPGPPSSGASERLVPEESYSRDQQPWGQSRGDENRGWMQRIRRRRRRRAALSGHLLDTEDNVPPWLPPHDIAPYVARNIRDAACRAVKQSHLQALSNLILDSGLDTQHILCFVMAARQRLQDIRRGPLVVEGGVGWRHWLLTSPSQSWPMGYRTATLRTLTPVPNRVGADSIMLTATFGCQNAARTLNTFSATVWTPPHAGPREQERYAREAEVRFLRGKWQRRYRRIYDLIELCGSLHHIWQNLLQTEENLLDFVRFMGVMSSCNNPAVNYWFHKTIGNFKPYYPWNAPPNENPYHARRGIKEHVIQNAFRKAQIQGLSMLATVGEPRGDATSETSSDEDTGRQGSDVELESSDDELPYIDPNMEPVQQRPVMFVSRVPAKKPRKLPWPTPKTHPVKRTNVKTSDRSDKAEAQSTPERPGPSEQSSVTVEPAHPTPVEMPMVILHQPPPVPKPVPVKPTPPPSRRRRGACVVYDDDVIEVIDVETTEDSSSVSQPNKPHRKHQDGFQRSGRRQKRAAPPTVSPSDTGPPAAGPPAAGPPAAGPHILTPPSARPRIMAPPVVRMFMRERQLPQSTGRKPQCFWEMRASREITQMQQEPSSHLQSATQPTMPRPSWVPSVCALSVMDAGKAQPIQSSHLSSMSPTQPISHEEQPRYEDPDAPLDLSLHPDVAAPPAPRAPYQGYQEQPAPQAPYQGYQEQPAPQAPYQGYQEQPAPQAPYQGYQEQPAPQAPYQGYQEQPPPQAPYQGYQEQPAPQAPYQGYQEPPAHGLQSSSYPGYAGPWTPRSQHPCYRHPWAPWSQDPVHGHTQGPWDPRAPHLPPQWDGSAGHGQDQVSQFPHLQSETGPPRLQLSSVPLVSSSAPSWSSPQPRAPIRPIPTRFPPPPMPLQDSMAVGCDSSGTACPSMPFASDYSQGAFTPLDINATTPKRPRVEESSHGPARCSQATAEAQEILSDNSEISVFPKDAKQTDYDASTESELD

SEQ ID NO: 16 - protein, 371 aa, Artificial
  EBV LMP1 Strain AG876
MEHDLERGPPGPRRPPRGPPLSSSLGLALLLLLLALLFWLYIVMSDWTGGALLVLYSFALILIIIILIIFIFRRDLLCPLGALCLLLLMITLLLIALWNLHGQALYLGIVLFIFGCLLVLGLWIYLLEILWRLGATIWQLLAFFLAFFLDLILLIIALYLQQNWWTLLVDLLWLLLFLAILIWMYYHGQRHSDEHHHDDSLPHPQQATDDSGHESDSNSNEGRHHLLVTGAGDGPPLCSQNLGAPGGGPDNGPQDPDNTDDNGPQDPDNTDDNGPQDPDNTDDNGPQDPDNTADNGPHDPLPHNPSDSAGNDGGPPNLTEEVENKGGDRGPPSMTDGGGGDPHLPTLLLGTSGSGGDDDDPHGPVQLSYYD

SEQ ID NO: 17 - protein, 386 aa, Artificial
  EBV LMP1 Strain B95-8
MEHDLERGPPGPRRPPRGPPLSSSLGLALLLLLLALLFWLYIVMSDWTGGALLVLYSFALMLIIIILIIFIFRRDLLCPLGALCILLLMITLLLIALWNLHGQALFLGIVLFIFGCLLVLGIWIYLLEMLWRLGATIWQLLAFFLAFFLDLILLIIALYLQQNWWTLLVDLLWLLLFLAILIWMYYHGQRHSDEHHHDDSLPHPQQATDDSGHESDSNSNEGRHHLLVSGAGDGPPLCSQNLGAPGGGPDNGPQDPDNTDDNGPQDPDNTDDNGPHDPLPQDPDNTDDNGPQDPDNTDDNGPHDPLPHSPSDSAGNDGGPPQLTEEVENKGGDQGPPLMTDGGGGHSHDSGHGGGDPHLPTLLLGSSGSGGDDDDPHGPVQLSYYD

SEQ ID NO: 18 - protein, 404 aa, Artificial
  EBV LMP1 Strain Cao
MERDLESAPPSAPRPPLGPPLSSSIGLALLLLLLALLFWLYIVMSDWTGGALLVLYSFALMLIIIILIIFIFRRDLLCPLGGLGLLLLMITLLLIALWNLHGQALYLGIVLFIFGCLLVFGIWIYFLEILWRLGATLWQLLAFILAFFLAIILLIIALYLQQNWWTLLVDLLWLLLFMAILIWMYYHGPRHTDEHHHDDSLPHPQQATDDSSHESDSNSNEGRHHLLVSGAGDGPPLCSQNLGAPGGGPDNGPQDPDNTDDNGPQDPDNTDDNGPQDPDNTDDNGPQDPDNTDDNGPQDPDNTDDNGPQDPDNTDDNGPQDPDNTDDNGPHDPLPHSPSDSAGNDGGPPNLTEEVANKGGDRGPPSMTDGGGGDPHLPTLLLGTSGSGGDDDDPHGPVQLSYYD

SEQ ID NO: 19 - protein, 366 aa, Artificial
  EBV LMP1 Strain GD1
MERDLERGPPGPPRPPLGPPLSSSIGLALLLLLLALLFWLYIVLSNWTGGALLVLYSFALMLIIIILIIFIFRRDLLCPLGGLGLLLLMVTLLLIALWNLHGQALYLGIVLFIFGCLLVLGLWIYFLEILWRLGATIWQLLAFILAFFLAIILLIIALYLQQNWWTLLVDLLWLLLFMAILIWMYFHGPRHTDEHHHDDSLPHPQQATDDSSHESDSNSNEGRHHLLVSGAGDGPPLCSQNLGAPGGGPDNGPQDPDNTDDNGPQDPDNTDDNGNTDDNGPQDPDNTDDNGPHDPLPHNPSDSAGNDGGPPNLTEEVENKGGDRGPPSMTDGGGGDPHLPTLLLGTSGSGGDDDDPHGPVQLSYYD

SEQ ID NO: 20 - protein, 386 aa, Artificial
  EBV LMP1 Strain Raji
MDLDLERGPPGPRRPPRGPPLSSSIGLALLLLLLALLFWLYIIMSNWTGGALLVLYAFALMLVIIILIIFIFRRDLLCPLGALCLLLLMITLLLIALWNLHGQALYLGIVLFIFGCLLVLGLWIYLLEILWRLGATIWQLLAFFLAFFLDIILLIIALYLQQNWWTLLVDLLWLLLFLAILIWMYYHGQRHSDEHHHDDSLPHPQQATDDSSNQSDSNSNEGRHLLLVSGAGDGPPLCSQNLGAPGGGPNNGPQDPDNTDDNGPQDPDNTDDNGPHDPLPQDPDNTDDNGPQDPDNTDDNGPHDPLPHNPSDSAGNDGGPPQLTEEVENKGGDQGPPLMTDGGGGHSHDSGHDGIDPHLPTLLLGTSGSGGDDDDPHGPVQLSYYD

SEQ ID NO: 21 - protein, 176 aa, Artificial
  EBV VP26 Strain B95-8
MARRLPKPTLQGRLEADFPDSPLLPKFQELNQNNLPNDVFREAQRSYLVFLTSQFCYEEYVQRTFGVPRRQRAIDKRQRASVAGAGAHAHLGGSSATPVQQAQAAASAGTGALASSAPSTAVAQSATPSVSSSISSLRAATSGATAAASAAAAVDTGSGGGGQPHDTAPRGARKKQ

SEQ ID NO: 22 - protein, 15 aa, Unknown
  Peptide
AYQAYAAPQLFPVSD

SEQ ID NO: 23 - protein, 15 aa, Unknown
  Peptide
QQPESLEECDSELEI

SEQ ID NO: 24 - protein, 15 aa, Unknown
  Peptide
EECDSELEIKRYKNR

SEQ ID NO: 25 - protein, 15 aa, Unknown
  Peptide
DSELEIKRYKNRVAS

SEQ ID NO: 26 - protein, 15 aa, Unknown
  Peptide
FKQLLQHYREVAAAK

SEQ ID NO: 27 - protein, 15 aa, Unknown
  Peptide
LLQHYREVAAAKSSE

SEQ ID NO: 28 - protein, 15 aa, Unknown
  Peptide
PSLDVDSIIPRTPDV

SEQ ID NO: 29 - protein, 15 aa, Unknown
  Peptide
DVDSIIPRTPDVLHE

SEQ ID NO: 30 - protein, 15 aa, Unknown
  Peptide
GLRALLARSHVERTT

SEQ ID NO: 31 - protein, 15 aa, Unknown
  Peptide
KTSLYNLRRGTALAI

SEQ ID NO: 32 - protein, 15 aa, Unknown
  Peptide
LYNLRRGTALAIPQC

SEQ ID NO: 33 - protein, 15 aa, Unknown
  Peptide
GTALAIPQCRLTPLS

SEQ ID NO: 34 - protein, 15 aa, Unknown
  Peptide
LAIPQCRLTPLSRLP

SEQ ID NO: 35 - protein, 15 aa, Unknown
  Peptide
RLTPLSRLPFGMAPG

SEQ ID NO: 36 - protein, 15 aa, Unknown
  Peptide
PLSRLPFGMAPGPGP

SEQ ID NO: 37 - protein, 15 aa, Unknown
  Peptide
DRPGPPALDDNMEEE

SEQ ID NO: 38 - protein, 15 aa, Unknown
  Peptide
ALDDNMEEEVPSTSV

SEQ ID NO: 39 - protein, 15 aa, Unknown
  Peptide
PPQPDLPGREAILRR

SEQ ID NO: 40 - protein, 15 aa, Unknown
  Peptide
PGREAILRRFPLDLR

SEQ ID NO: 41 - protein, 15 aa, Unknown
  Peptide
EAILRRFPLDLRTLL

SEQ ID NO: 42 - protein, 15 aa, Unknown
  Peptide
KWRLQTLAAGWPMGY

SEQ ID NO: 43 - protein, 15 aa, Unknown
  Peptide
LQTLAAGWPMGYQAY

SEQ ID NO: 44 - protein, 15 aa, Unknown
  Peptide
YTDHQTTPTFVHLQA

SEQ ID NO: 45 - protein, 15 aa, Unknown
  Peptide
LQATLGCTGGRRCHV

SEQ ID NO: 46 - protein, 15 aa, Unknown
  Peptide
TLGCTGGRRCHVTFS

SEQ ID NO: 47 - protein, 15 aa, Unknown
  Peptide
TFSAGTFKLPRCTPG

SEQ ID NO: 48 - protein, 15 aa, Unknown
  Peptide
PRCTPGDRQWLYVQS

SEQ ID NO: 49 - protein, 15 aa, Unknown
  Peptide
NPRYSIFFDYMAIHR

SEQ ID NO: 50 - protein, 15 aa, Unknown
  Peptide
YSIFFDYMAIHRSLT

SEQ ID NO: 51 - protein, 15 aa, Unknown
  Peptide
FFDYMAIHRSLTKIW

SEQ ID NO: 52 - protein, 15 aa, Unknown
  Peptide
LGFLQRTDLSYIKSF

SEQ ID NO: 53 - protein, 15 aa, Unknown
  Peptide
LQRTDLSYIKSFVSD

SEQ ID NO: 54 - protein, 15 aa, Unknown
  Peptide
QAWNAGFLRGRAYGI

SEQ ID NO: 55 - protein, 15 aa, Unknown
  Peptide
FLRGRAYGIDLLRTE

SEQ ID NO: 56 - protein, 15 aa, Unknown
  Peptide
YGIDLLRTEGEHVEG

SEQ ID NO: 57 - protein, 15 aa, Unknown
  Peptide
VSRGGPKVKRPPIFI

SEQ ID NO: 58 - protein, 15 aa, Unknown
  Peptide
GGPKVKRPPIFIRRL

SEQ ID NO: 59 - protein, 15 aa, Unknown
  Peptide
STYGTPRPPVPKPRP

SEQ ID NO: 60 - protein, 15 aa, Unknown
  Peptide
PIVRPWEPSLTQAAG

SEQ ID NO: 61 - protein, 15 aa, Unknown
  Peptide
ALERPVYPKPVRPAP

SEQ ID NO: 62 - protein, 15 aa, Unknown
  Peptide
RPVYPKPVRPAPPKI

SEQ ID NO: 63 - protein, 15 aa, Unknown
  Peptide
LGYTLHGLNHPGVPV

SEQ ID NO: 64 - protein, 15 aa, Unknown
  Peptide
RWKLLSSCRSWRMGY

SEQ ID NO: 65 - protein, 15 aa, Unknown
  Peptide
LLSSCRSWRMGYRTH

SEQ ID NO: 66 - protein, 15 aa, Unknown
  Peptide
MGYRTHNLKVNSFES

SEQ ID NO: 67 - protein, 15 aa, Unknown
  Peptide
NLKVNSFESGGDNVH

SEQ ID NO: 68 - protein, 15 aa, Unknown
  Peptide
IETAFLMARRARSLS

SEQ ID NO: 69 - protein, 15 aa, Unknown
  Peptide
AFLMARRARSLSAER

SEQ ID NO: 70 - protein, 15 aa, Unknown
  Peptide
HIREWFRQCTGRPKA

SEQ ID NO: 71 - protein, 15 aa, Unknown
  Peptide
PKAAKPWLRAHPVAI

SEQ ID NO: 72 - protein, 15 aa, Unknown
  Peptide
AKPWLRAHPVAIPYD

SEQ ID NO: 73 - protein, 15 aa, Unknown
  Peptide
EIDLAYARGQAMNIE

SEQ ID NO: 74 - protein, 15 aa, Unknown
  Peptide
LPYNPTVYGRPAVFD

SEQ ID NO: 75 - protein, 15 aa, Unknown
  Peptide
VYGRPAVFDRKSDAK

SEQ ID NO: 76 - protein, 15 aa, Unknown
  Peptide
HRKKKAARTEQPRAT

SEQ ID NO: 77 - protein, 15 aa, Unknown
  Peptide
PQQPRAGRRGPCVFT

SEQ ID NO: 78 - protein, 15 aa, Unknown
  Peptide
AAPRQWPMPLRPIPM

SEQ ID NO: 79 - protein, 15 aa, Unknown
  Peptide
RQWPMPLRPIPMRPL

SEQ ID NO: 80 - protein, 15 aa, Unknown
  Peptide
LRPIPMRPLRMQPIP

SEQ ID NO: 81 - protein, 15 aa, Unknown
  Peptide
IPMRPLRMQPIPFNH

SEQ ID NO: 82 - protein, 15 aa, Unknown
  Peptide
RPLRMQPIPFNHPVG

SEQ ID NO: 83 - protein, 15 aa, Unknown
  Peptide
PTWAQIGHIPYQPTP

SEQ ID NO: 84 - protein, 15 aa, Unknown
  Peptide
PATMQTPPRAPTPMS

SEQ ID NO: 85 - protein, 15 aa, Unknown
  Peptide
LKLQAALERQAAAGW

SEQ ID NO: 86 - protein, 15 aa, Unknown
  Peptide
LGLGDIAVSSPSSSE

SEQ ID NO: 87 - protein, 15 aa, Unknown
  Peptide
WGQSRGDENRGWMQR

SEQ ID NO: 88 - protein, 15 aa, Unknown
  Peptide
DENRGWMQRIRRRRR

SEQ ID NO: 89 - protein, 15 aa, Unknown
  Peptide
IRRRRRRRAALSGHL

SEQ ID NO: 90 - protein, 15 aa, Unknown
  Peptide
RRRRRAALSGHLLDT

SEQ ID NO: 91 - protein, 15 aa, Unknown
  Peptide
LDTEDNVPPWLPPHD

SEQ ID NO: 92 - protein, 15 aa, Unknown
  Peptide
WLPPHDITPYTARNI

SEQ ID NO: 93 - protein, 15 aa, Unknown
  Peptide
ITPYTARNIRDAACR

SEQ ID NO: 94 - protein, 15 aa, Unknown
  Peptide
RNIRDAACRAVKQSH

SEQ ID NO: 95 - protein, 15 aa, Unknown
  Peptide
VMAARQRLQDIRRGP

SEQ ID NO: 96 - protein, 15 aa, Unknown
  Peptide
LVAEGGVGWRHWLLT

SEQ ID NO: 97 - protein, 15 aa, Unknown
  Peptide
EGGVGWRHWLLTSPS

SEQ ID NO: 98 - protein, 15 aa, Unknown
  Peptide
RHWLLTSPSQSWPMG

SEQ ID NO: 99 - protein, 15 aa, Unknown
  Peptide
QSWPMGYRTATLRTL

SEQ ID NO: 100 - protein, 15 aa, Unknown
  Peptide
PMGYRTATLRTLTPV

SEQ ID NO: 101 - protein, 15 aa, Unknown
  Peptide
YRTATLRTLTPVPNR

SEQ ID NO: 102 - protein, 15 aa, Unknown
  Peptide
AREAEVRFLRGKWQR

SEQ ID NO: 103 - protein, 15 aa, Unknown
  Peptide
HHIWQNLLQTEENLL

SEQ ID NO: 104 - protein, 15 aa, Unknown
  Peptide
LLQTEENLLDFVRFM

SEQ ID NO: 105 - protein, 15 aa, Unknown
  Peptide
TEENLLDFVRFMGVM

SEQ ID NO: 106 - protein, 15 aa, Unknown
  Peptide
NLLDFVRFMGVMSSC

SEQ ID NO: 107 - protein, 15 aa, Unknown
  Peptide
DFVRFMGVMSSCNNP

SEQ ID NO: 108 - protein, 15 aa, Unknown
  Peptide
SSCNNPAVNYWFHKT

SEQ ID NO: 109 - protein, 15 aa, Unknown
  Peptide
AVNYWFHKTIGNFKP

SEQ ID NO: 110 - protein, 15 aa, Unknown
  Peptide
YWFHKTIGNFKPYYP

SEQ ID NO: 111 - protein, 15 aa, Unknown
  Peptide
HKTIGNFKPYYPWNA

SEQ ID NO: 112 - protein, 15 aa, Unknown
  Peptide
FKPYYPWNAPPNENP

SEQ ID NO: 113 - protein, 15 aa, Unknown
  Peptide
RRGIKEHVIQNAFRK

SEQ ID NO: 114 - protein, 15 aa, Unknown
  Peptide
IKEHVIQNAFRKAQI

SEQ ID NO: 115 - protein, 15 aa, Unknown
  Peptide
QNAFRKAQIQGLSML

SEQ ID NO: 116 - protein, 15 aa, Unknown
  Peptide
SSVSQPNKPHRKHQD

SEQ ID NO: 117 - protein, 15 aa, Unknown
  Peptide
GPHIVTPPSARPRIM

SEQ ID NO: 118 - protein, 15 aa, Unknown
  Peptide
APPVVRMFMRERQLP

SEQ ID NO: 119 - protein, 15 aa, Unknown
  Peptide
MFMRERQLPQSTGRK

SEQ ID NO: 120 - protein, 15 aa, Unknown
  Peptide
FWEMRAGREITQMQQ

SEQ ID NO: 121 - protein, 15 aa, Unknown
  Peptide
PGYAGPWTPRSQHPC

SEQ ID NO: 122 - protein, 15 aa, Unknown
  Peptide
WTPRSQHPCYRHPWA

SEQ ID NO: 123 - protein, 15 aa, Unknown
  Peptide
RSQHPCYRHPWAPWS

SEQ ID NO: 124 - protein, 15 aa, Unknown
  Peptide
FPHLQSETGPPRLQL

SEQ ID NO: 125 - protein, 15 aa, Unknown
  Peptide
ETGPPRLQLSLVPLV

SEQ ID NO: 126 - protein, 15 aa, Unknown
  Peptide
RAPIRPIPTRFPPPP

SEQ ID NO: 127 - protein, 15 aa, Unknown
  Peptide
LLLLALLFWLYIVMS

SEQ ID NO: 128 - protein, 15 aa, Unknown
  Peptide
LEMLWRLGATIWQLL

SEQ ID NO: 129 - protein, 15 aa, Unknown
  Peptide
LAILIWMYYHGQRHS

SEQ ID NO: 130 - protein, 15 aa, Unknown
  Peptide
LIWMYYHGQRHSDEH

SEQ ID NO: 131 - protein, 15 aa, Unknown
  Peptide
MYYHGQRHSDEHHHD

SEQ ID NO: 132 - protein, 15 aa, Unknown
  Peptide
MARRLPKPTLQGRLE

SEQ ID NO: 133 - protein, 15 aa, Unknown
  Peptide
FCYEEYVQRTFGVPR

SEQ ID NO: 134 - protein, 15 aa, Unknown
  Peptide
TFGVPRRQRAIDKRQ

SEQ ID NO: 135 - protein, 15 aa, Unknown
  Peptide
RASVAGAGAHAHLGG

SEQ ID NO: 136 - DNA, 18 bp, Artificial Sequence
  forward Primer for EBER 1
tcccgggtacaagtcccg

SEQ ID NO: 137 - DNA, 20 bp, Artificial Sequence
  reverse primer for EBER-1
tgaccgaagacggcagaaag

SEQ ID NO: 138 - DNA, 28 bp, Artificial Sequence
  Detection probe
tggtgaggacggtgtctgtggttgtgtt

SEQ ID NO: 139 - DNA, 19 bp, Artificial Sequence
  forward primer
ccagagcgcagctatcggt

SEQ ID NO: 140 - DNA, 21 bp, Artificial Sequence
  reverse primer
cacgtttggcatggatagcac

SEQ ID NO: 141 - DNA, 28 bp, Artificial Sequence
  Detection probe
gcaagtgaggcctatctggttggccttt

SEQ ID NO: 142 - DNA, 20 bp, Artificial Sequence
  forward primer for EBNA-1
tacaggacctggaaatggcc

SEQ ID NO: 143 - DNA, 19 bp, Artificial Sequence
  reverse primer for EBNA-1
tctttgaggtccactgccg

SEQ ID NO: 144 - DNA, 26 bp, Artificial Sequence
  Detection probe for EBNA-1
agggagacacatctggaccagaaggc

SEQ ID NO: 145 - DNA, 29 bp, Artificial Sequence
  forward primer for BZLF-1
aaatttaagagatcctcgtgtaaaacatc

SEQ ID NO: 146 - DNA, 20 bp, Artificial Sequence
  reverse primer for BZLF-1
cgcctcctgttgaagcagat

SEQ ID NO: 147 - DNA, 28 bp, Artificial Sequence
  Detection probe for BZLF-1
ataatggagtcaacatccaggcttgggc

SEQ ID NO: 148 - protein, 22 aa, artificial
  EBNA1_EBVB9_394 - 414 peptide
PPRRPPPGRRPFFHPVGEADYF

SEQ ID NO: 149 - protein, 15 aa, artificial
  EBNA1_EBVB9_0394 peptide
PPRRPPPGRRPFFHP

SEQ ID NO: 150 - protein, 15 aa, artificial
  EBNA1_EBVB9_0397 peptide
RPPPGRRPFFHPVGE

SEQ ID NO: 151 - protein, 15 aa, artificial
  EBNA1_EBVB9_0400
PGRRPFFHPVGEADY

SEQ ID NO: 152 - protein, 22 aa, artificial
  EBNA6_EBVB9_741 - 779
PPRRPPPGRRPFFHPVGEADYF

SEQ ID NO: 153 - protein, 15 aa, artificial
  EBNA6_EBVB9_0748 peptide
QGYQEPPAPQAPYQG

SEQ ID NO: 154 - protein, 15 aa, artificial
  EBNA6_EBVB9_0751 peptide
QEPPAPQAPYQGYQE

SEQ ID NO: 155 - protein, 15 aa, artificial
  EBNA6_EBVB9_0754 peptide
PAPQAPYQGYQEPPP

(19) United States
(12) Patent Application Publication
(10) Pub. No.: US 2016/0153994 A1
(43) Pub. Date: Jun. 2, 2016

(54) EBV PROTEINS AS MARKERS IN METHODS OF DIAGNOSING CHRONIC FATIGUE SYNDROME (CFS)

(71) Applicants: JPT Peptide Technologies GmbH, Berlin (DE); Chartié-Universitatsmedizin Berlin (Charité), Berlin (DE)

(72) Inventors: Carmen Scheibenbogen, Berlin (DE); Hans-Dieter Volk, Berlin (DE); Josef Priller, Berlin (DE); Klemens Ruprecht, Berlin (DE); Ulf Reimer, Berlin (DE); Holger Wenschuh, Berlin (DE)

(21) Appl. No.: 14/896,261

(22) PCT Filed: Jun. 10, 2014
(86) PCT No.: PCT/EP2014/061984

§ 371 Date: Dec. 4, 2015

(30) Foreign Application Priority Data
Jun. 10, 2013 (EP) 13171260.6

(51) Int. Cl. G01N 33/569; C12Q 1/70

(52) U.S. Cl. CPC G01N 33/56994; C12Q 1/705; C12Q 2600/158; C12Q 2600/112; G01N 2333/05; G01N 2469/10

(57) ABSTRACT

The present invention relates to an in vitro method for diagnosing Chronic Fatigue Syndrome by determining the presence, absence or the amount of at least one marker characteristic of an Epstein-Barr virus (EBV) infection in a sample obtained from the body of an individual. Specifically, the marker characteristic of EBV infection is selected from the group consisting of EBNA1, EBNA3, EBNA4, EBNA6, BZLF1, LMP1 and VP26. Furthermore, the invention relates to a device for the diagnosis of Chronic Fatigue Syndrome, wherein the device comprises a solid phase having immobilized thereon at least one marker or protein fragment thereof, wherein the marker is selected from the group consisting of EBNA1, EBNA3, EBNA4, EBNA6, BZLF1, LMP1 and VP26. In addition, the invention relates to the use of at least one marker or a protein fragment thereof for diagnosis of Chronic Fatigue, wherein the marker is selected from the group consisting of EBNA1 EBNA3, EBNA4, EBNA6, BZLF1, LMP1 and VP26.

The present application claims the right of priority to European Patent Application 13171260.6, filed 10 Jun. 2013, the entire content of which is incorporated herein by reference.

### FIELD OF THE INVENTION

The present invention relates to the development of an in vitro method for diagnosing Chronic Fatigue Syndrome (CFS) by determining the presence, absence or the amount of at least one specific marker characteristic of an Epstein-Barr virus (EBV) infection in a sample obtained from the body of an individual. In addition, the invention relates to a device for the diagnosis of Chronic Fatigue Syndrome using at least one specific marker characteristic of Epstein-Barr virus.

### BACKGROUND

Chronic Fatigue Syndrome (CFS) is a complex disease characterized by overwhelming fatigue accompanied by physical symptoms resembling a severe flu-like illness, including muscle pain, impaired memory or mental concentration, insomnia, and post-exertion malaise. CFS can persist for years and is difficult to diagnose since no diagnostic tests are available. At present, CFS is diagnosed mainly according to the Fukuda-Criteria which were established in 1994. These guidelines are based on the fulfillment of two major criteria: chronic fatigue causing substantial reduction of occupational, personal and social activities, lasting more than 6 months and the exclusion of associated medical and psychiatric conditions (Fukuda et al. Ann. Intern. Med. 1994, 121: 953-959). In 2003, the Center of Disease Control published recommendations for use of the definition, standardization of classification instruments and study design to improve the identification of CFS in patients. Even though CFS has an estimated prevalence of 0.3% in the general population and is relatively frequent, it is often not diagnosed correctly. So far, CFS is a purely clinical diagnosis, which can only be provided by experienced physicians. The distinction between CFS and other physical disorders, such as depression or burn-out, is often difficult. At present, there is neither a diagnostic marker nor approved drug to treat CFS.

Numerous viral or microbial pathogens have been considered as possible causes of CFS, since many patients report an acute onset of symptoms with an infectious disease (Prins, et al., 2006, Lancet, 367(9507):346-355). Several human viruses (e.g. Epstein Barr Virus (EBV), Human Herpes Virus (HHV), Xenotropic murine leukemia virus-related virus (XMRV), Enteroviruses) have been associated with CFS, but their pathogenic relationship to the syndrome has not been demonstrated conclusively (Frémont M et al., 2009, In Vivo, 23(2):209-13; Wallace HL, 1999, Clin. Diagn. Lab. Immunol., 6(2):216-23; Lombardi et al., 2009, Science, 326(5952):585-589; van Kuppeveld, 2010, BMJ, 25:340). In this regard, various attempts have been made to diagnose CFS using the connection to pathogens such as Herpes Viruses, however none of these have so far led to a satisfactory result.

In U.S. Pat. No. 6,894,056 B2, it is disclosed that CFS may be detected by combining the steps of monitoring the patients for T-wave abnormalities with an electrocardiograph to document the cardiac pathology considered the basis for CFS and evaluating the patient for serologic evidence of EBV and HCMV infection by measuring antibodies to these viruses. For the EBV, the capsid antigen (VCA) IgM and total early antigens (EA, EA-D) were measured, to see if an active or persistent multiplication of this virus could be determined, which was possible in a subset of patients having cardiac pathology indicative of CFS. However, a direct relationship between EBV and CFS could not be observed as healthy control individuals also tested positive for these EBV-specific antibodies. Rather, the serologic evidence provided a basis for the differential treatment of CFS patients with antiviral medications.

Based on these studies, the same inventor continued the research, which was published as WO 2009/054957 A2. Again, this document relates to possible methods for diagnosing and treating chronic fatigue syndrome in different subclasses of patients. Here, patients were tested for the presence of serum antibodies to herpes viruses such as e.g. EBV (VCA, EA-D, IgM-antigen-viral capsid p18), human cytomegalovirus (HCMV) and human herpes virus 6 (HHV6). Patients being only seropositive for EBV represented a subclass of CFS patients, since other subgroups of patients were tested seropositive for only HCMV or HHV6. However, CFS patients were not compared to control groups, but rather subclassified to allow differential pharmacological treatment of CFS, so that while this publication indicates a possible connection between CFS and EBV, HHV6 or HCMV, it does not provide a method for diagnosing CFS. In fact, these studies imply that EBV may only be involved in a subset of CFS patients.

Thus despite this research into the possible causes of CFS and possible treatment approaches, there remains a need for a diagnostic marker or test to differentiate CFS from other physical disorders with similar symptoms. So far there is no diagnostic marker available that can distinguish between a healthy individual and an individual affected by CFS.

### SUMMARY OF THE INVENTION

The present invention has been made by the comparison of samples from healthy individuals to samples from individuals suffering from CFS as diagnosed by experienced physicians. The inventors surprisingly found that in patients suffering from CFS, the immune response to several markers characteristic of EBV is different from that found in healthy individuals. Based on these differences, the present invention provides an in vitro method for diagnosing Chronic Fatigue Syndrome. The method of the invention comprises determining the presence, absence or the amount of at least one marker characteristic of Epstein-Barr virus (EBV) infection in a sample obtained from the body of an individual, wherein the presence of Chronic Fatigue Syndrome can be concluded from the presence, absence or the amount of the marker in the sample.

In the most preferred embodiment, the marker is selected from the group consisting of Epstein-Barr nuclear antigen 1 (EBNA1), Epstein-Barr nuclear antigen 3 (EBNA3), Epstein-Barr nuclear antigen 4 (EBNA4), Epstein-Barr nuclear antigen 6 (EBNA6), Trans-activator protein BZLF1 of Epstein Barr Virus (BZLF1), LMP1 and VP26. In one embodiment of the invention, the absence of one, two or all of the markers EBNA1, EBNA3, EBNA4 and EBNA6 in the sample indicates the presence of Chronic Fatigue Syndrome. In another embodiment, the presence of one or both markers BZLF1 and VP26 indicates the presence of Chronic Fatigue Syndrome.

The method of the invention also allows for a distinction between Chronic Fatigue Syndrome and a different physical disorder, such as depression or burnout syndrome, by determining the presence, absence or amount of at least one of the markers EBNA3, EBNA4, and EBNA6.

In the methods of the present invention, the presence, absence of the amount of the marker can be determined directly or indirectly by means of a suitable assay. Specifically, the assay can determine the presence, absence or amount of each marker directly by measuring the presence, absence or amount of the corresponding protein or a fragment thereof or the corresponding nucleotide encoding the protein, or indirectly by measuring the presence, absence of amount of an immune response to the corresponding protein or a fragment thereof.

In a preferred embodiment of the invention, the immune response is determined by an immunoassay. Specifically, the immunoassay can comprise at least one of the following protein fragments. The protein fragments can be between 4 and 70 amino acids in length from the specified regions of the following sequences: 80-105 of SEQ ID NO.: 1; amino acid 158-245 of SEQ ID NO.: 1; amino acid 80-105 of SEQ ID NO.: 2; amino acid 158-245 of SEQ ID NO.: 2; amino acid 80-105 of SEQ ID NO.: 3; amino acid 158-245 of SEQ ID NO.: 3; amino acid 479-555 of SEQ ID NO.: 4; amino acid 479-555 of SEQ ID NO.: 5; amino acid 479-555 of SEQ ID NO.: 6; amino acid 1-30 of SEQ ID NO.: 7; amino acid 76-110 of SEQ ID NO.: 7; amino acid 154-431 of SEQ ID NO.: 7; amino acid 489-548 of SEQ ID NO.: 7; amino acid 784-828 of SEQ ID NO.: 7; amino acid 1-30 of SEQ ID NO.: 8; amino acid 77-111 of SEQ ID NO.: 8; amino acid 155-432 of SEQ ID NO.: 8; amino acid 503-567 of SEQ ID NO.: 8; amino acid 803-828 of SEQ ID NO.: 8; amino acid 1-30 of SEQ ID NO.: 9; amino acid 77-111 of SEQ ID NO.: 9; amino acid 155-432 of SEQ ID NO.: 9; amino acid 503-567 of SEQ ID NO.: 9; amino acid 794-819 of SEQ ID NO.: 9; amino acid 161-204 of SEQ ID NO.: 10; amino acid 230-259 of SEQ ID NO.: 10; amino acid 290-351 of SEQ ID NO.: 10; amino acid 383-450 of SEQ ID NO.: 10; amino acid 524-549 of SEQ ID NO.: 10; amino acid 614-719 of SEQ ID NO.: 10; amino acid 790-815 of SEQ ID NO.: 10; amino acid 922-946 of SEQ ID NO.: 10; amino acid 161-204 of SEQ ID NO.: 11; amino acid 230-259 of SEQ ID NO.: 11; amino acid 290-351 of SEQ ID NO.: 11; amino acid 383-450 of SEQ ID NO.: 11; amino acid 524-549 of SEQ ID NO.: 11; amino acid 614-711 of SEQ ID NO.: 11; amino acid 782-807 of SEQ ID NO.: 11; amino acid 914-938 of SEQ ID NO.: 11; amino acid 161-204 of SEQ ID NO.: 12; amino acid 230-259 of SEQ ID NO.: 12; amino acid 290-351 of SEQ ID NO.: 12; amino acid 383-450 of SEQ ID NO.: 12; amino acid 524-549 of SEQ ID NO.: 12; amino acid 614-711 of SEQ ID NO.: 12; amino acid 782-807 of SEQ ID NO.: 12; amino acid 914-938 of SEQ ID NO.: 12; amino acid 53-204 of SEQ ID NO.: 13; amino acid 236-306 of SEQ ID NO.: 13; amino acid 519-575 of SEQ ID NO.: 13; amino acid 632-696 of SEQ ID NO.: 13; amino acid 855-980 of SEQ ID NO.: 13; amino acid 53-204 of SEQ ID NO.: 14; amino acid 236-306 of SEQ ID NO.: 14; amino acid 518-543 of SEQ ID NO.: 14; amino acid 605-669 of SEQ ID NO.: 14; amino acid 785-903 of SEQ ID NO.: 14; amino acid 53-204 of SEQ ID NO.: 15; amino acid 236-306 of SEQ ID NO.: 15; amino acid 518-543 of SEQ ID NO.: 15; amino acid 570-634 of SEQ ID NO.: 15; amino acid 802-920 of SEQ ID NO.: 15; amino acid 26-51 of SEQ ID NO.: 16; amino acid 122-147 of SEQ ID NO.: 16; amino acid 173-204 of SEQ ID NO.: 16; amino acid 26-51 of SEQ ID NO.: 17; amino acid 122-147 of SEQ ID NO.: 17; amino acid 173-204 of SEQ ID NO.: 17; amino acid 26-51 of SEQ ID NO.: 18; amino acid 122-147 of SEQ ID NO.: 18; amino acid 173-204 of SEQ ID NO.: 18; amino acid 26-51 of SEQ ID NO.: 19; amino acid 122-147 of SEQ ID NO.: 19; amino acid 173-204 of SEQ ID NO.: 19; amino acid 26-51 of SEQ ID NO.: 20; amino acid 122-147 of SEQ ID NO.: 20; amino acid 173-204 of SEQ ID NO.: 20; amino acid 1-21 of SEQ ID NO.: 21; or amino acid 50-99 of SEQ ID NO.: 21, or any one of SEQ ID NOs.:22-135. In a preferred embodiment, the fragments have a length of 15 amino acids. In a further preferred embodiment, the fragments correspond to one or more of SEQ ID NOs.: 22-135, SEQ ID NOs.: 149-151 or SEQ ID NOs: 153-155. The sequences referred to above are summarized in Table 1 of the detailed description.

In one embodiment of the invention, an immunoassay is used to determine the presence, absence or amount of at least one marker, and the protein fragments are immobilized on a solid phase and brought into contact with the sample obtained from the body of the individual, to determine the immune response (antibody response) of the individual. In a preferred embodiment, the immune response to at least one of the markers which are selected from the group consisting of EBNA1, EBNA3, EBNA4, EBNA6, BZLF1, LMP1 and VP26 is determined.

In some embodiments of the method of the invention, the method further comprises determining the number of memory B cells of the individual. In this embodiment, the total number of memory B cells and the number of EBV-specific memory B cells can be determined, wherein a reduction in the number of EBV-specific memory B cells relative to the number of EBV specific memory B-cells from an EBV seropositive control group of healthy individuals indicates the presence of Chronic Fatigue Syndrome.

In another embodiment of the invention, the method further comprises determining the response of TNF-alpha or interferon gamma-producing memory T cells specific to EBV. In this embodiment a reduction of TNF-alpha or interferon gamma-producing memory T cells relative to the number of TNF-alpha or interferon gamma-producing memory T cells of an EBV seropositive control group of healthy individuals indicates the presence of Chronic Fatigue Syndrome.

In the methods of the invention, the sample obtained from the body of an individual is selected from the group consisting of a blood sample, a saliva sample, a urine sample, and a sample from a pharyngeal wash.

The present invention also encompasses a device for the diagnosis of Chronic Fatigue Syndrome, wherein the device comprises a solid phase having immobilized thereon a protein comprising or consisting of at least one marker characteristic of a Epstein-Barr virus (EBV) infection or a protein fragment thereof. In a preferred embodiment, the marker is selected from the group consisting of EBNA1, EBNA3, EBNA4, EBNA6, BZLF1, LMP1 and VP26.

Finally, the invention relates to a protein comprising or consisting at least one marker or protein fragment thereof for use in the diagnosis of Chronic Fatigue Syndrome, wherein the marker is selected from the group consisting of EBNA1 EBNA3, EBNA4, EBNA6, BZLF1, LMP1 and VP26.

In one embodiment of the invention, the preferred marker is EBNA3, EBNA4 or EBNA6 or a fragment thereof.

### BRIEF DESCRIPTION OF THE DRAWINGS

The invention will be better understood with reference to the detailed description when considered in conjunction with the non-limiting examples and the accompanying drawings, which are:

FIG. 1: (A) Serum IgG titer were assessed for healthy donors (Control) and CFS patients by ELISA for EBV-VCA IgG (control n=57, CFS n=63),
(B) EBNA IgG (control n=57, CFS n=63),
(C) EBV-VCA IgM (control n=57, CFS n=63) and
(D) CMV IgG (control n=32, CFS n=41) and (E) CMV IgM (control n=32, CFS n=41). Statistical analysis was performed using the two-tailed Mann-Whitney-U test and for EBNA IgG and EBV-VCA IgM Fisher's exact one-tailed test for association analysis with *p<0.05

FIG. 2: (A) Serum IgG titer were assessed for CFS patients by ELISA for EBV-IgG and EBV-EBNA-1 IgG (n=397), (B) EBNA-1 negative (neg, n=7) and positive (pos, n=8) CFS patients from the cohort of A were compared for total IgG, and (C) the frequencies of CD19⁺ B-cells, (D) IgD⁺ IgM⁺ CD27⁺ marginal zone B-cells, and (D) IgD⁻ CD27⁺ class-switched memory B-cells. Statistical analysis was performed using the two-tailed Mann-Whitney-U test with ***p<0.001 and n.s.—not significant.

FIG. 3: Replicating EBV can be detected in CFS patients. (A) EBV DNA was analyzed via nested real-time PCR in total PBMCs of healthy donors (Controls, n=29) and CFS patients (n=26) for EBER-1. (B) The same analysis as described in A was performed for EBV-BZLF-1 in PBMCs of CFS patients and healthy controls (n=27). Correlation of BZLF-1 and EBER-1 is displayed for responders of CFS patients (white circles) and responders of healthy controls (black circles) that showed detectable copy numbers of either BZLF-1 or EBER-1. EBER-1 and BZLF-1 copies were calculated in accordance to Namalwa standard. Statistical analysis was performed using the one-tailed Mann-Whitney-U test with *p<0.05. Correlation is displayed as linear regression analysis with n.s.—not significant.

FIG. 4: EBV specific memory B-cells are reduced in CFS patients. (A) Compared are the frequencies of EBV-specific memory B-cells between healthy donors (Controls) and CFS patients after 7 days of polyclonal stimulation. Secreted total IgG was assessed with the ELISpot assay after 6 h of incubation on a 96 well plate coated with anti-IgG (n=10), (B) EBV (n=10), HSV and CMV lysate (control n=6, CFS n=4), (C) VCA protein (n=10), and (D) EBNA-1 protein (n=10). IgG secreting B-cells are shown as frequencies from 1*10⁶ seeded cells. Statistical analysis was performed using the two-tailed Mann-Whitney-U test with *p<0.05, **p<0.001 and n.s.—not significant.

FIG. 5: CFS patients show diminished cytokine response against EBV. Whole blood of healthy donors (Control) and CFS patients was analyzed by CBA for (A) IFN-γ production after stimulation with either EBV lysate (control n=29, CFS n=22), EBNA-1 peptide (control n=24, CFS n=11) and SEB (control n=21, CFS n=11) in cell supernatants and (B) after EBV lysate stimulation for TNF-α (control n=29, CFS n=22), IL-2 (control n=29, CFS n=22) and IL-10 (control n=25, CFS n=13). Statistical analysis was performed using the two-tailed Mann-Whitney-U test with *p<0.05 and **p<0.01.

FIG. 6 CFS patients show reduced EBV specific memory T-cell response. Comparison of cytokine producing CD8⁺ (upper panels) and CD4⁺ T-cells (lower panels) of CFS patients (n=15) and healthy donors (Controls, n=9) after 10 days of antigen specific stimulation with EBNA-1. Cytokines were assessed by intracellular staining of isolated PBMCs after incubation with Brefeldin A for 16 h. Boolean gating strategy was applied to analyze (A) IFN-γ, TNF-α and IL-2 single, (B) IFN-γ/TNF-α double and (C) IFN-γ/TNF-α/IL-2 triple cytokine producing T-cells. Statistical analysis was performed using the two-tailed Mann-Whitney-U test with *p<0.05 and n.s.—not significant.

FIG. 7: Heatmap of BLRF2 in Normal, Depression and CFS patients. The x-axis represents the samples used in the screening experiment, the y axis shows peptides representing a scan through the respective protein with a peptide length of 15 amino acids and an overlap of 11 amino acids (N-terminus—bottom, C-terminus—top).

FIG. 8: Heatmap of BZLF1 in Normal, Depression and CFS patients.

FIG. 9 Heatmap of EBNA1 in Normal, Depression and CFS patients.

FIG. 10: Heatmap of EBNA3 in Normal, Depression and CFS patients.

FIG. 11: Heatmap of EBNA4 in Normal, Depression and CFS patients.

FIG. 12: Heatmap of EBNA6 in Normal, Depression and CFS patients.

FIG. 13: Heatmap of LMP1 in Normal, Depression and CFS patients.

FIG. 14: Heatmap of VP26 in Normal, Depression and CFS patients.

FIG. 15: Heatmap of signal intensities for selected peptides for all patients Selection criterion is a p-value<0.01 for the signal intensities of CFS-patients vs. controls and CFS-patients vs. patients diagnosed with depression.

FIG. 16A-16I: Boxplots of signal intensities for individual peptides fragments with a p-value<0.01: CFS patients vs. Depression patients

### DETAILED DESCRIPTION OF THE INVENTION

As already mentioned above, the present invention relies on the comparison of samples from healthy individuals to samples from individuals suffering from CFS as diagnosed by experienced physicians. While former studies, such as those cited above, indicated a possible involvement of EBV, HHV6 and CMV in CFS and allowed for the classification of subgroups of CFS patients for differential treatment with antivirals, no significant differences between the immune response to specific antigens of these viruses (for EBV: VCA, EA-D, IgM-antigen-viral capsid p18) was found between CFS and healthy individuals.

Thus the technical problem of the present invention is to develop an in vitro method for diagnosing CFS in a sample obtained from the body of an individual. Surprisingly, contrary to previous findings, it was found that in patients suffering from CFS, the immune response to several markers characteristic of EBV is different from that found in healthy individuals and in individuals with other physical disorders such as depression. In the present application it was found that the analysis of specific antibodies and of cellular defects is suitable for the diagnosis of CFS. Especially, microarrays using protein fragments are excellently suitable for the diagnosis of CFS. Below, the experiments performed to arrive at the invention are summarized briefly after which each of the methods for diagnosing CFS of the present invention will be described in more detail. In this experiment, it was found that a subset of CFS patients shows abnormal EBV serology.

In a preliminary study, EBV-specific antibody titers (EBNA-1 IgG, VCA IgG and IgM), were determined and analysed via ELISA (Example 1). The most prominent finding was the deficiency of EBNA1-IgG antibodies (≦20 U/ml) in 12.7% of EBV-seropositive CFS patients, in contrast to 3.5% of healthy EBV-seropositive controls (p<0.01). In addition, it was found that while titers of VCA IgG were similar in CFS patients and controls, 17.5% of the CFS patients but only 3.5% of the controls had elevated VCA IgM levels. Thus Elevated EBV-VCA IgM and lack of EBNA IgG were detected in different patient subsets accounting for 30% of CFS patients with an abnormal EBV serology. As a comparison, HSV and CMV IgG were measured and were detected in all groups, revealing no difference between CFS patients and healthy controls. This finding led the inventors to focus on EBV. In order to determine if CFS-patients bear a higher EBV load than healthy subjects. In a further preliminary study, real-time PCR (Example 2) was used to detect viral transcripts in samples obtained from the body of individuals. However, it was found that this method did not allow the unambiguous differentiation of CFS patients from healthy individuals.

As mentioned above, EBV infects B-cells and remains latent in these cells after the acute infection has passed, so that EBV-specific memory B-cells are present in the host after the infection. This allowed for the investigation of EBV-specific memory B-cells after polyclonal stimulation in CFS-patients and healthy controls (Example 3), and the ELISASpot assay was used to enumerate IgG-producing cells. US patent application 2010/0267009 A1 describes an “ELISASpot” assay for differentiation between acute infections and latent or overcome infections, which can be EBV, that comprises incubating eukaryotic cells with an antigen and testing for cells secreting antigen-specific antibodies. In the Experiments described in Example 3, was found that EBV specific memory B-cells are low in number or absent in most CFS patients. No difference between CFS patients and healthy controls was detected for total IgG producing B-cells quantified by ELISpot assay. Remarkably, the in vitro differentiation of memory B cells into both EBNA-1 and VCA-antibody secreting plasma cells was strongly diminished in patients with CFS.

After this, Antigen-specific T-cell responses were measured in a subset of patients by cytokine production in culture supernatants (Examples 4 and 5). Here it was found that peripheral blood mononuclear cells (PBMCs) of patients with CFS produced significantly lower levels of TNF-α and IFN-γ in response to EBV lysate and certain EBV peptides. In vitro expanded EBNA-specific memory T cells had a significantly diminished fraction of multifunctional CD4 and CD8 T cells. U.S. Pat. No. 7,897,357 B2 and US patent application 2010/0035282 A1 describe an assay for the detection of chemokines having activity that is upregulated by Th-1 cytokines (such IFN-γ) and chemokines that upregulate the activity of Th-1 cytokines (such as IFN-γ). The detection of the chemokine monokine induced by gamma interferon (MIG) provides a measure of the biological effect of IFN-γ rather than direct quantitation of IFN-γ or IFN-γ secreting cells per se.

As already mentioned above, both the ELISPOT described in US 2010/0267009 A1 as well as the EBV-T-cell assay (U.S. Pat. No. 7,897,357 B2; and US patent application 2010/0035282 A1) are standard methods, which were developed for the detection of an infection-specific immune response. Other protein/peptide based techniques such as Luminex technology to detect antibodies or flow cytometry using biotin-labeled proteins or peptides to detect antibody-bearing memory B cells could also be used in this context. Taken together, the Experiments described above and in Examples 1-5 provide evidence for an exhausted B and T cell memory response against EBV in patients with CFS. This suggests a chronic low level of EBV replication in patients with CFS.

These findings prompted the inventors to test the samples from CFS patients, healthy volunteers and depression patients for an immune response to several specific EBV-antigens known to be characteristic for latent EBV infection, as well as several antigens characteristic for acute EBV infection by means of microarrays in order to identify specific markers for CFS (Example 6). The strongest markers for CFS identified are EBNA3, EBNA4 and EBNA6, though differences were also found in the immune response to BZLF1, EBNA1, LMP1 and VP26.

Based on these differences, the present invention provides an in vitro method for diagnosing Chronic Fatigue Syndrome. Specifically, three different tests for diagnosing CFS are provided. As summarized above, the primary method for diagnosing CFS involves determining the presence, absence or amount of at least one marker for EBV selected from the group of EBNA1, EBNA3, EBNA4, EBNA6, BZLF1, LMP1 and VP26. Of course this method also relates to determining the presence, absence or amount of two, three, four, five, six or all of these markers. After this, the presence, absence or amount of further markers for EBV can also be determined.

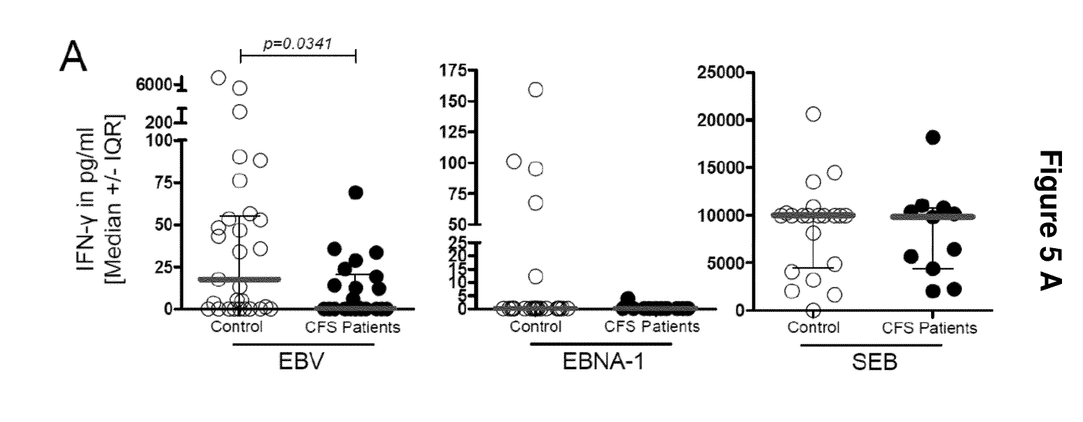

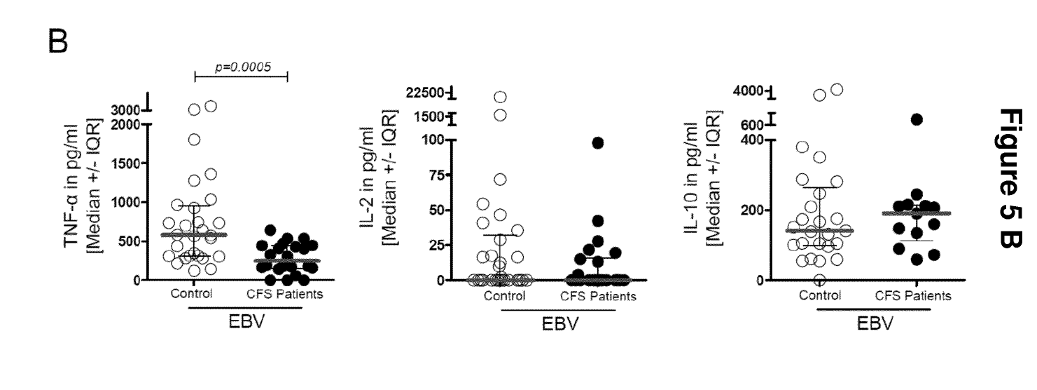

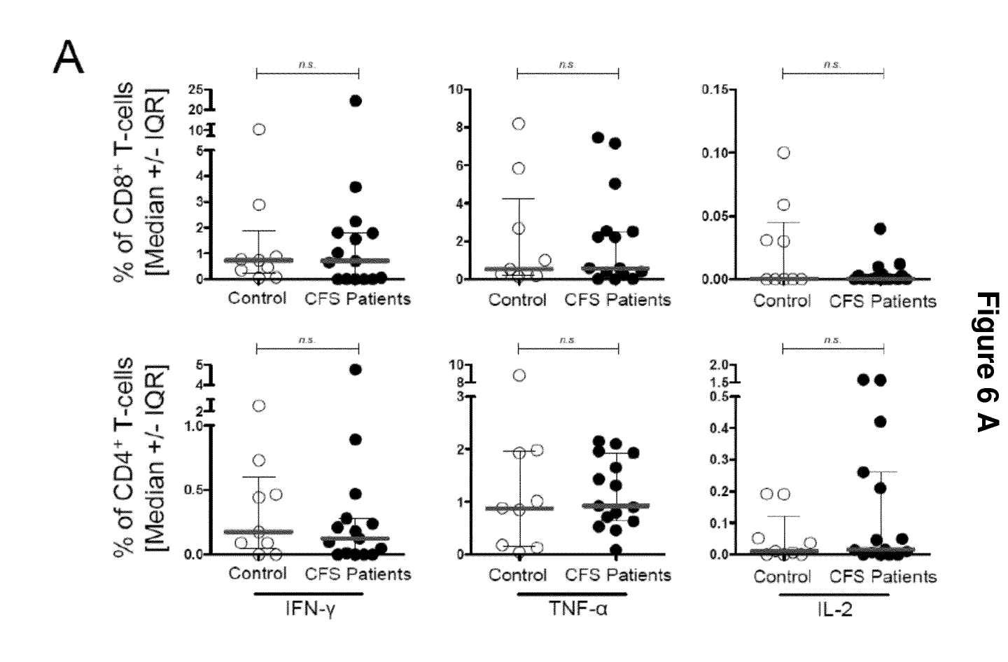

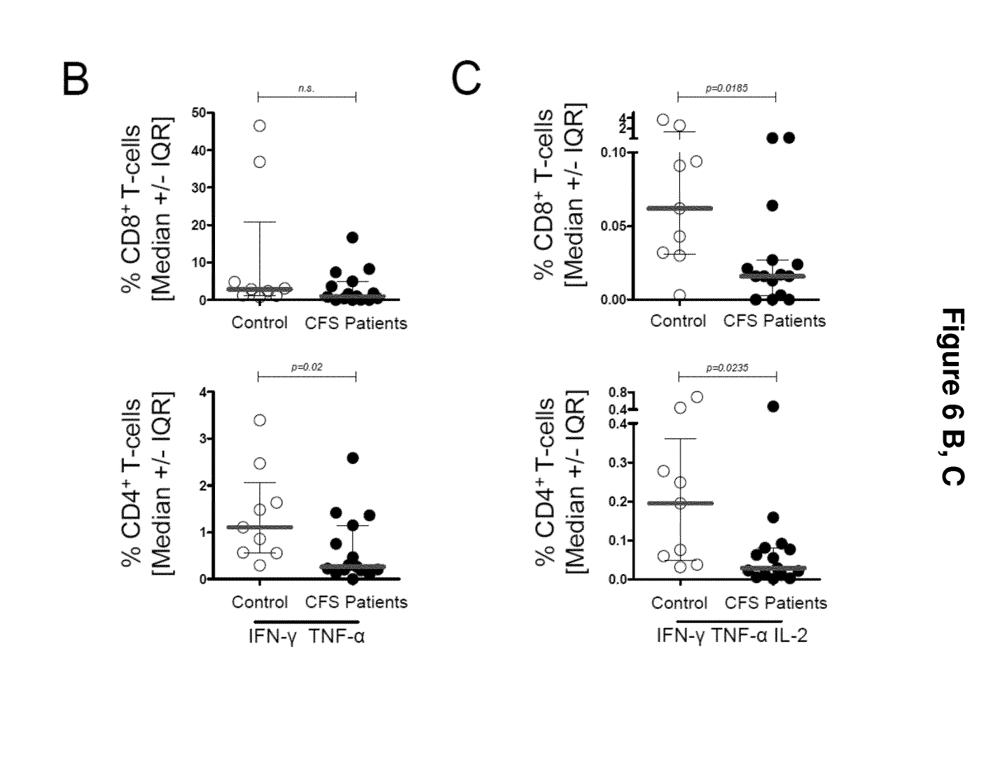

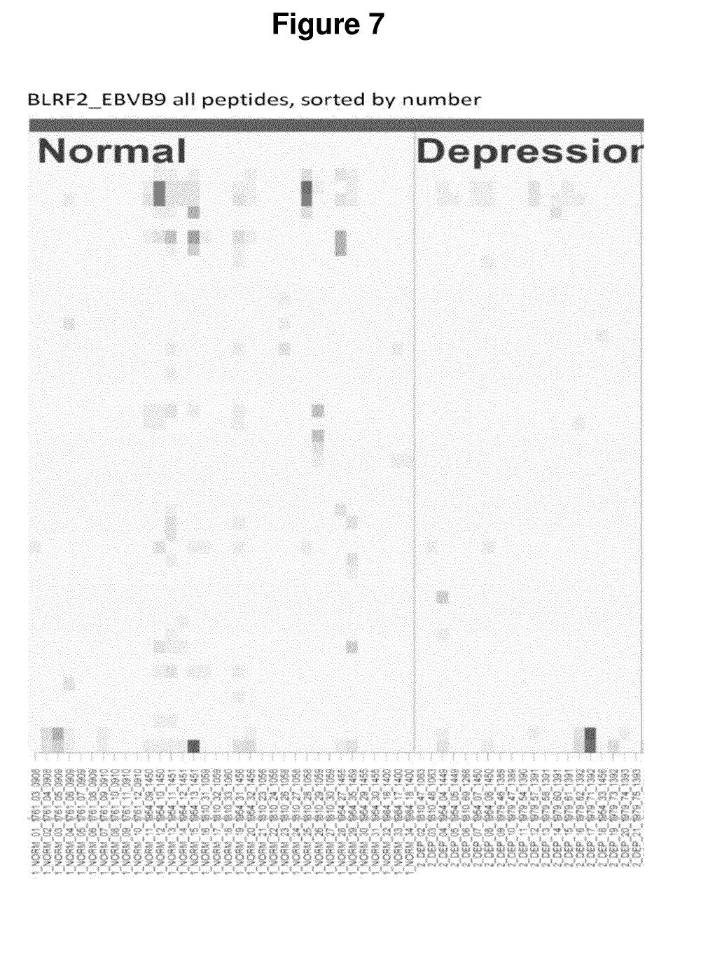

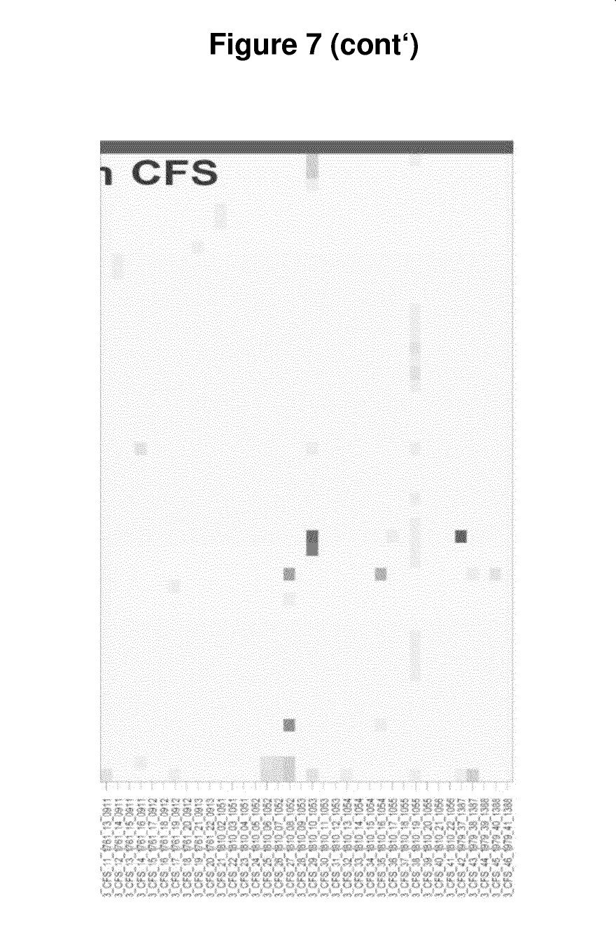

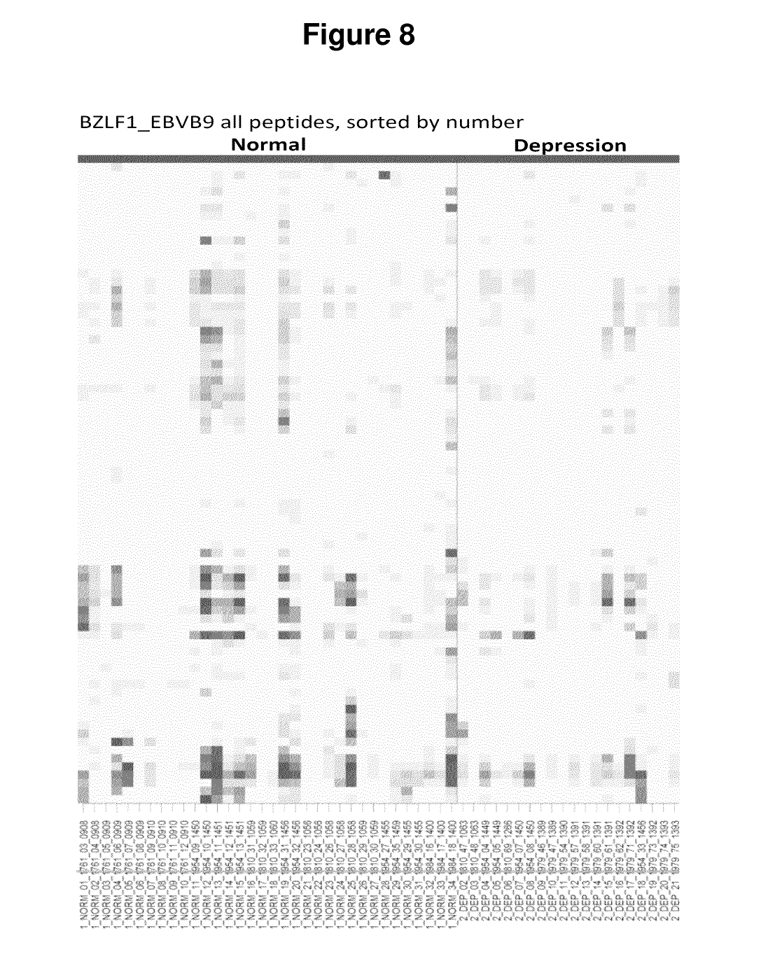

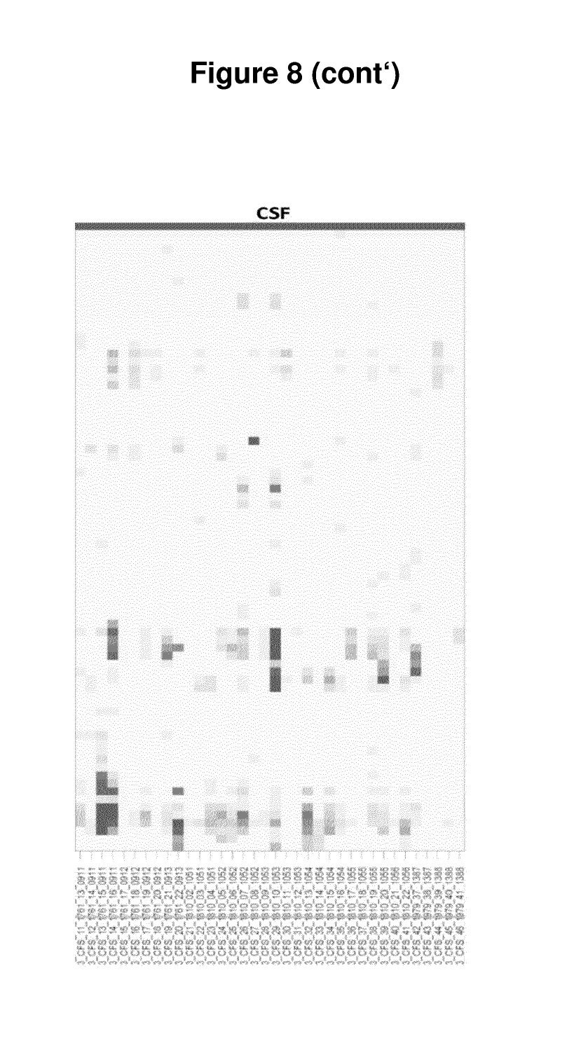

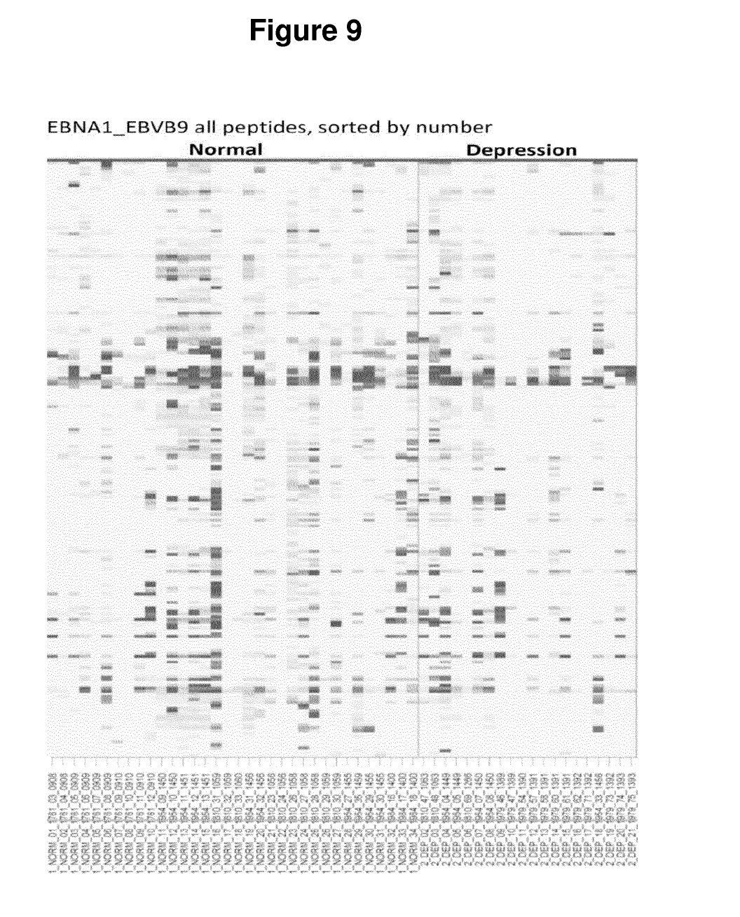

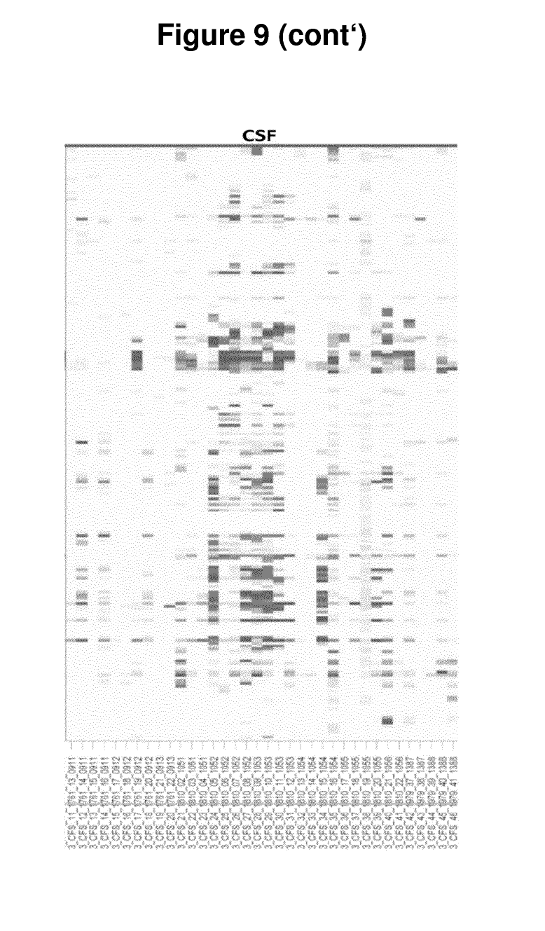

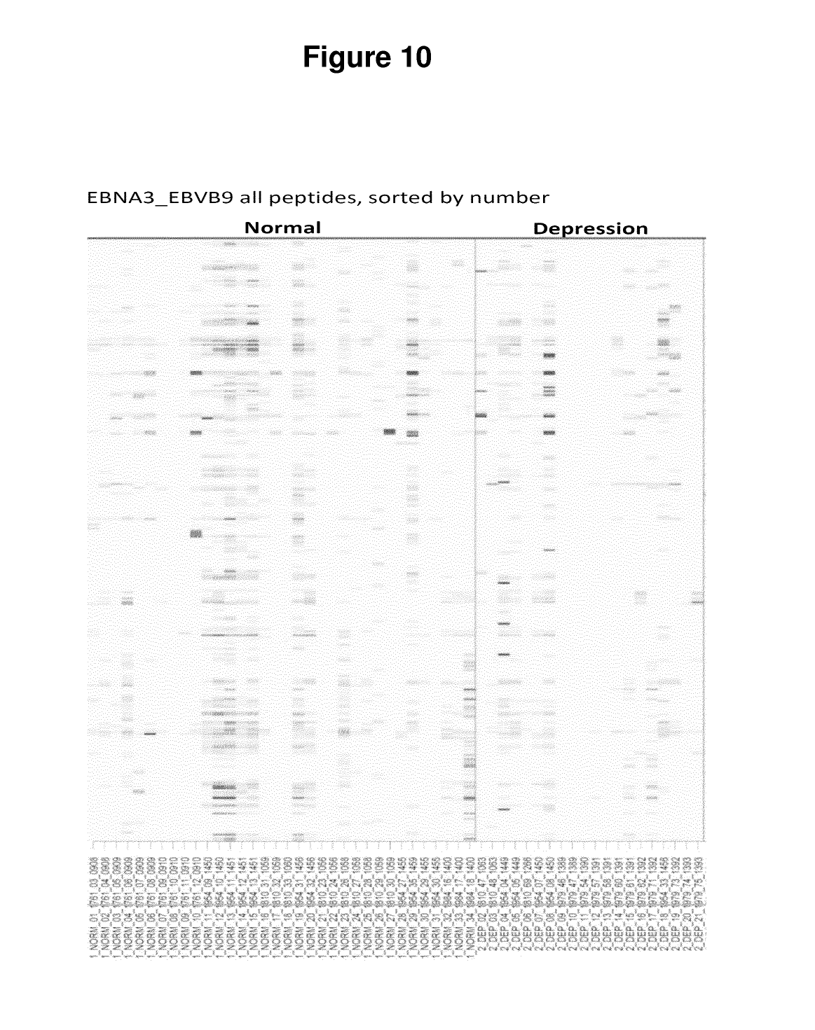

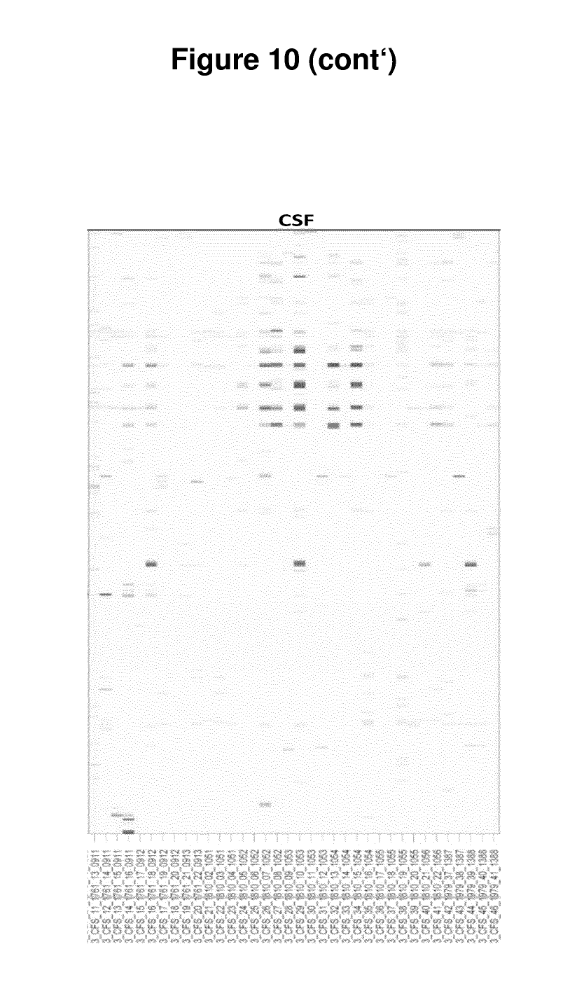

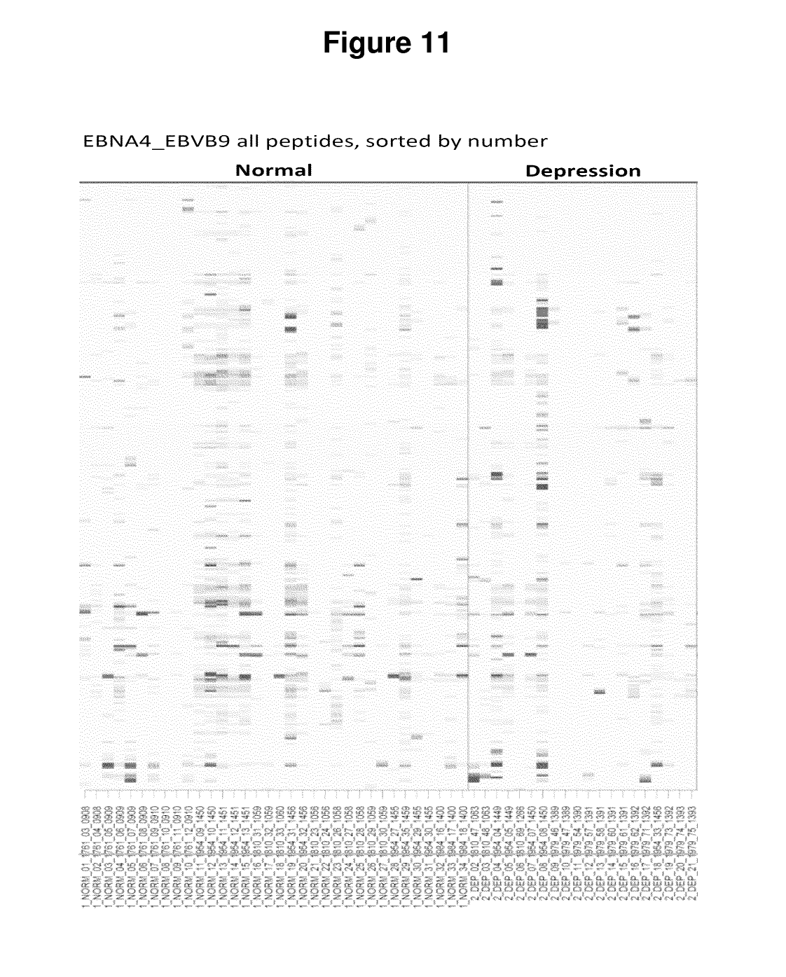

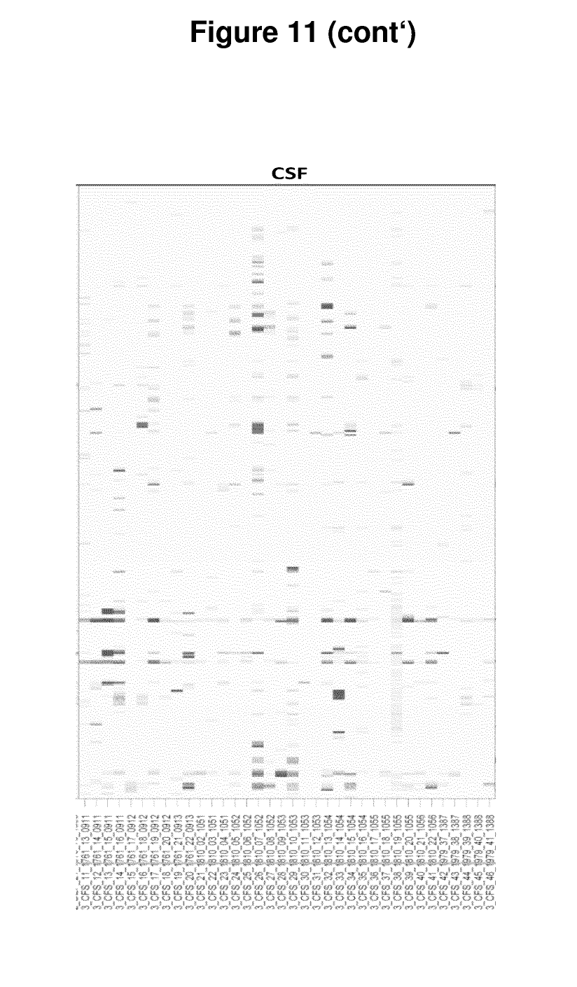

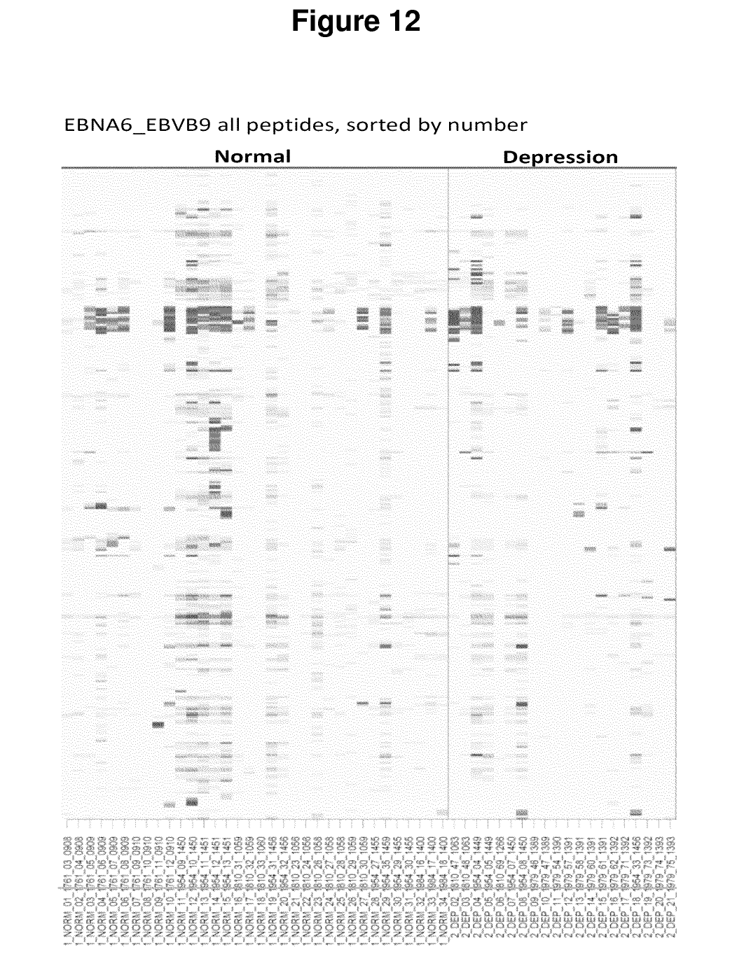

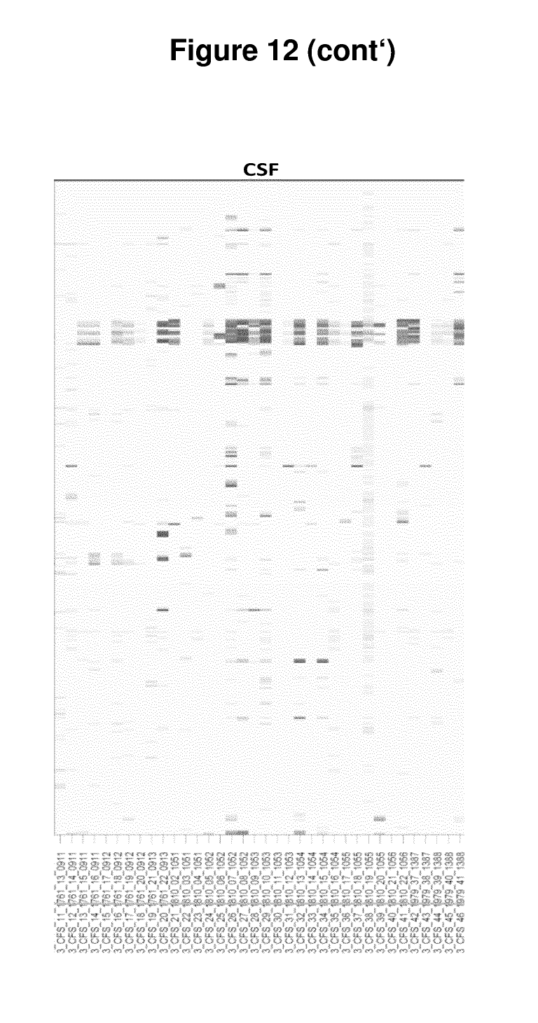

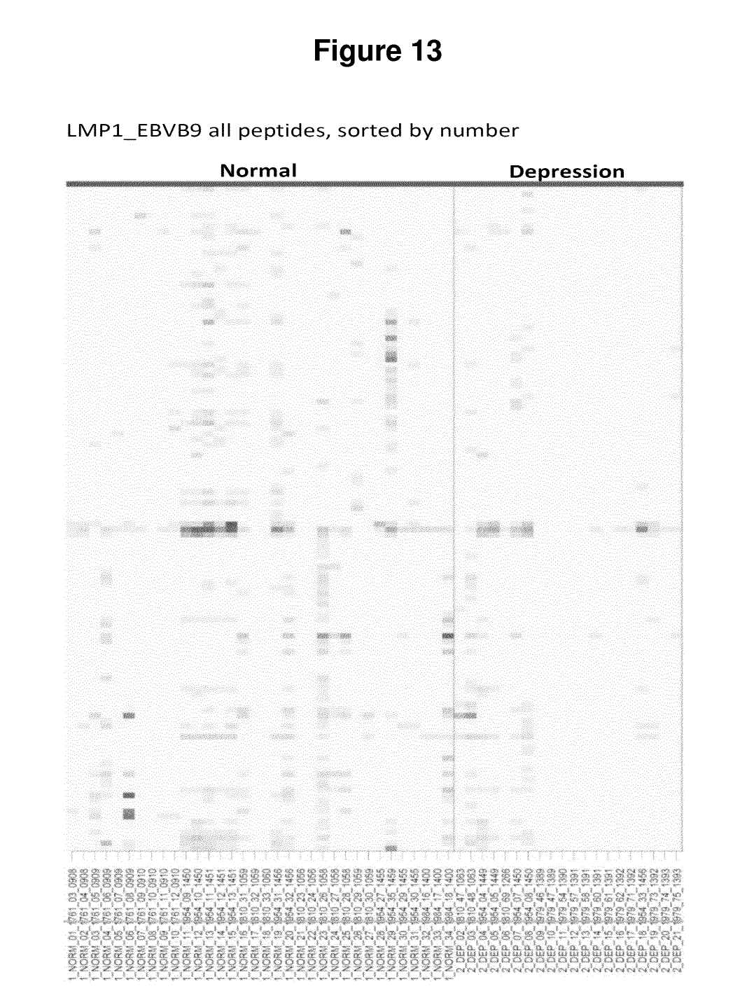

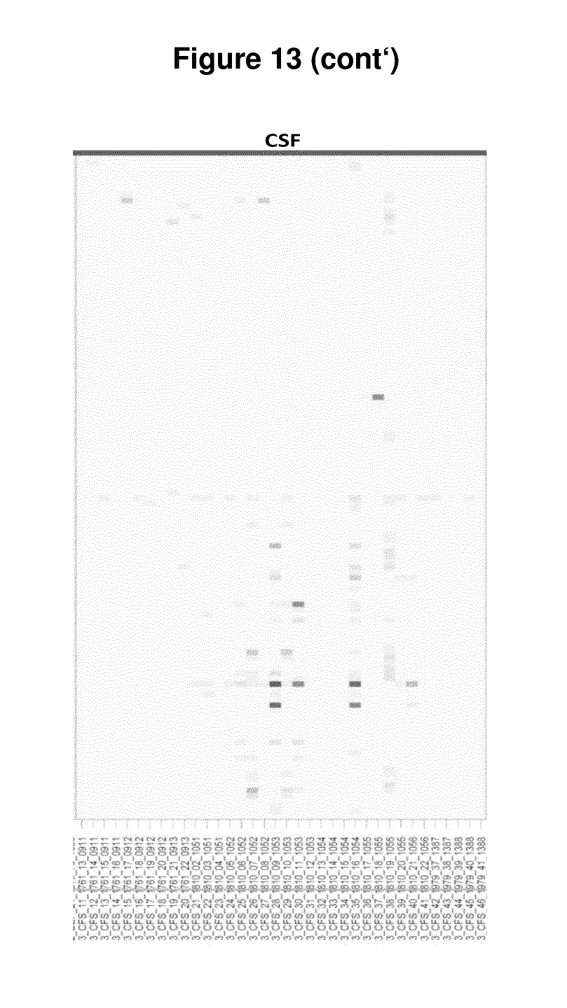

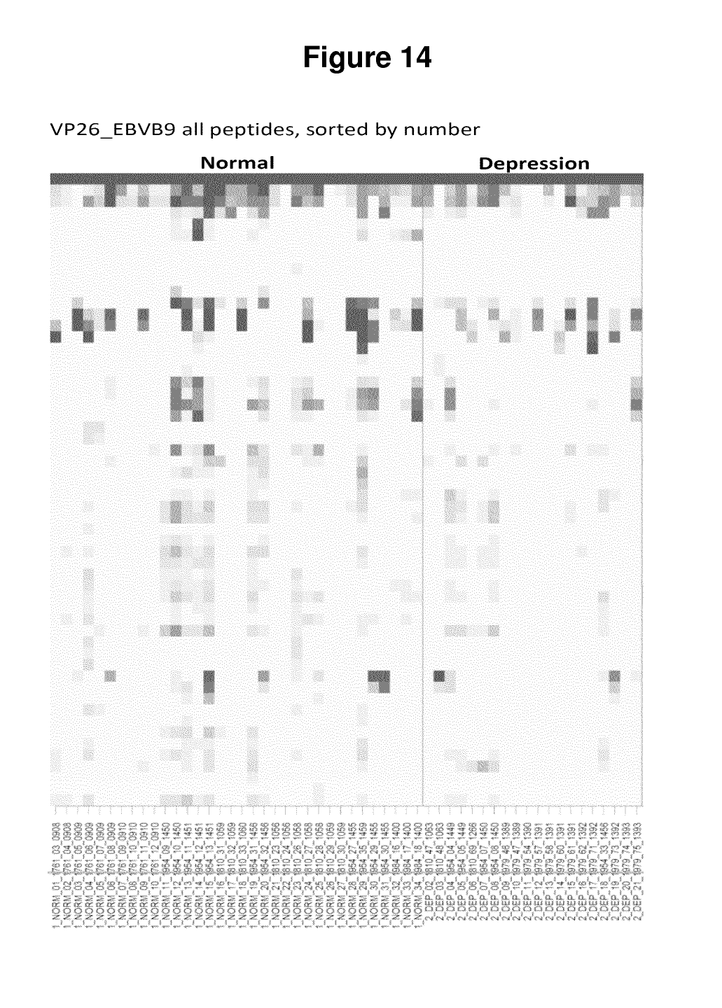

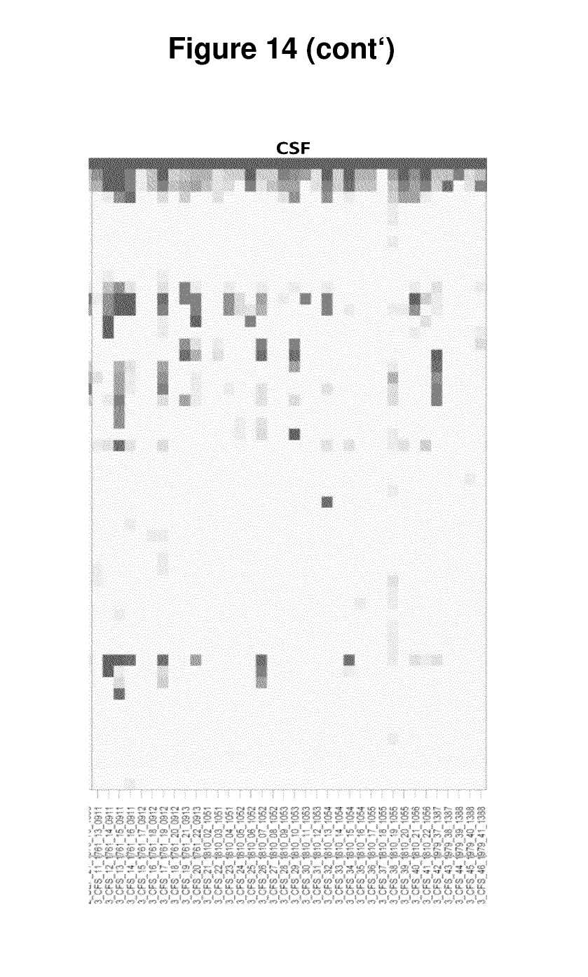

In this regard it should be noted that the presence, absence or amount of the marker can be determined by any assay suitable to detect the presence or absence of the marker directly or indirectly. The marker is measured “directly” by measuring the presence, absence or amount of the corresponding protein or a fragment thereof or the corresponding nucleotide encoding the protein in the sample. The marker can also be measured “indirectly” by measuring the presence, absence of amount of an immune response to the corresponding protein or a fragment thereof. In the methods described in Example 6, the absence of the markers of interest in CFS patients was determined indirectly using an immunoassay with fragments of the proteins of interest bound to microarrays. The microarray libraries were then incubated with the samples from patients with CFS, patients with depression and healthy volunteers as described in Example 6. The heatmaps of FIGS. 7 to 15 show the distinct immune response (seroreactivity) of individuals with CFS as opposed to the healthy controls and the patients suffering from depression. This shows that the specific markers of the invention also allow for the differentiation between CFS and other physical disorders such as depression.

Specifically, in patients suffering from CFS, the immune response to at least one of EBNA1, EBNA3, EBNA4 or EBNA6 was absent or reduced. In some cases, the immune response to 2, 3 or all of these markers was absent or reduced. Thus the absence of an immune response to the full length protein or fragments of EBNA1, EBNA3, EBNA4 or EBNA6 (or to two, three or all of these) indicates that the patient is suffering from CFS. Likewise, the presence of an immune response to the full length protein or fragments of BZLF1 or VP26 is indicative of CFS.

Another sensitive approach to determine the B cell response against EBV is by analysing the number of EBV-specific B cells, which are severely reduced or absent in most patients with CFS. In a similar manner the memory T cell response against EBV is reduced in patients with CFS as outlined in detail below.

After this first step, the total number of memory B cells and the number of EBV-specific memory B cells is determined and compared to the number of EBV-specific memory B cells from a seropositive control group of healthy individuals. A reduction in the number of EBV-specific memory B cells is indicative of CFS.

Specifically, the ELISpot assay was used as a readout to analyze antigen-specific memory B-cells. It was demonstrated that CFS—patients show significantly reduced frequencies of EBV-specific memory B-cells after polyclonal stimulation. For two EBV-specific antigens used in these experiments (EBV-VCA protein and EBV-EBNA-1 protein) and EBV-lysate patients displayed a reduced response compared to healthy controls. Also the total amount of IgG secreting cell is similar in healthy controls and CFS-patients. This strongly suggests that overall humoral immune response in CFS-patients is not altered.

To assess whether the reduction of EBV-specific memory B-cells is due to T-cells with cytotoxic activity that recognize the MHC-EBV-peptide complex on the B-cells surface enriched B-cells were cultivated without T-cells. Frequencies of EBV-specific memory B-cells resulting from this stimulation are comparable to the results obtained with polyclonal stimulation. Even though frequencies detected after stimulation were higher, it has to be considered that enriched B-cells were used for this protocol. Overall same frequencies were found for EBV-lysate specific B-cell as for the polyclonal stimulation. These findings suggest that disruption of memory B-cells by T-cells in the course of differentiation is unlikely. Higher frequencies would have been expected for the stimulation with soluble CD40 ligand, since this stimulation is independent of donor-derived T-cells.

A further method for diagnosing EBV involves determining the response of TNF-alpha or interferon gamma-producing memory T cells, wherein the reduction of TNF-alpha or interferon gamma-producing memory T cells of an EBV seropositive control group relative to the number of TNF-alpha or interferon gamma-producing memory T cells of healthy individuals indicates the presence of Chronic Fatigue Syndrome. This method can be performed as described in the prior art mentioned above or according to Example 4. In this regard it was found that the PBMCs of patients with CFS produced significantly lower levels of TNFα and IFNγ in response to EBV lysate and certain EBV peptides. In vitro expanded EBNA-specific memory T cells had a significantly diminished fraction of multifunctional CD4 and CD8 T cells.

This “exhausted” T-cell response is often observed in HIV and HCV infection (Rajziewicz et al., 2007, J. Virol. 81: 2545-2553). It has been studied thoroughly in LCMV-infection in mice and mechanisms for the underlying causes have been proposed. T-cell dysfunction is controlled by cytokines and negative regulatory receptors. It is proposed that persistence and continuous exposure to antigen hyperimmune activation together with several other factors (i.e. presence of IL-10 and upregulation of immune-suppressor molecules) gradually drives T cells into exhaustion (El-Far et al., 2008, Curr. HIV/AIDS Rep. 5: 13-19). Cellular exhaustion as a consequence of persistent viral stimulus in CFS-patients has been described in the past though only IFNγ was assessed (Klimas et al., 1990, J Clin Microbiol 28: 1403-1410; Visser et al., 1998, J. Infect. Dis., 177: 451-454).

In a further embodiment, the invention relates to a device for the diagnosis of CFS comprising a solid phase comprising at least one marker for EBV or a fragment thereof immobilized on it. In a preferred embodiment, the marker for EBV selected from the group of EBNA1, EBNA3, EBNA4, EBNA6, BZLF1, LMP1 and VP26 or any suitable fragment thereof. In the most preferred embodiment, at least one of the markers is EBNA3, EBNA4 or EBNA6 or a fragment thereof. Suitable fragments are described below, most preferable are those provided in SEQ ID NOs.:22-135, SEQ ID NOs. 149-151 and SEQ ID NOs: 153-155. The device of the invention can be any solid phase test device such as a microarray, an immunoassay such as an ELISA plate, or a polymer bead, to which the fragments can be attached by methods known in the art, for example following the instructions published elsewhere (Barouch et al., (2013) J Infect Dis 207: 248-256).

These embodiments are characterized and described herein, illustrated in the Examples, and reflected in the claims. In the following section, the terms used in the claims and in the description are defined.

In the context of the present invention, the term “in vitro method” refers to a method that is performed outside of the human body, in contrast to an in vivo method. The method of the present invention is a diagnostic assay, so that the word “method” could be replaced with “test” or “assay”.

The aim of the in vitro method of the invention is to diagnose Chronic Fatigue Syndrome. The expression “diagnose” means identifying CFS in a patient based on the methods of the invention.

As already mentioned above, the term “Chronic Fatigue Syndrome” (CFS) refers to a clinical condition of uncertain cause that is characterized by overwhelming fatigue, as diagnosed mainly according to the Fukuda-Criteria which were established in 1994. These guidelines are based on the fulfillment of two major criteria: chronic fatigue causing substantial reduction of occupational, personal and social activities, lasting more than 6 months and the exclusion of associated medical and psychiatric conditions (Fukuda et al. Ann. Intern. Med. 1994, 121: 953-959).

The term “determining the presence, absence or amount” reflects the use of the method to investigate the presence or absence of the marker and, if present, determining the amount thereof. The presence, absence or amount of the marker can be determined by an assay suitable to detect the presence or absence of the marker directly or indirectly, such as a peptide microarray, ELISA, Luminex or Luminex-like platforms, ELISPOT assay for antibody and B cell responses and specific cytokine release in response to the marker assessed by ELISA or flow cytometry for T cell responses both ex vivo or after in vitro expansion.

The marker can be measured “directly” by measuring the presence, absence or amount of the corresponding protein or a fragment thereof or the corresponding nucleotide encoding the protein in the sample. The marker can also be measured indirectly by measuring the presence, absence of amount of an immune response to the corresponding protein or a fragment thereof. The immune response can be measured from a body fluid sample (for example, a plasma, serum or whole blood sample, or a lymph sample) obtained from the individual who is suspected of suffering from CFS. The reactivity of the immunoglobulins (antibodies) that are present in this body sample can then be analyzed in a solid phase assay (cf. also Example 6 in this regard). For this purpose, fragments of the full length marker proteins considered here (i.e. fragments of EBNA1, EBNA3, EBNA4, EBNA6, BZLF1, LMP1 and VP26) can be immobilized on a solid support. Since the amino acid sequence of all the marker proteins used herein are known, it is possible to divide the linear polypeptide sequence of these marker proteins into short fragments of a length of, for example, about 10, 15, 20, 30, or 40 amino acids (the fragments may also have any length within this range, for example 15 or 25 amino acids). These fragments are then synthesized and immobilized on a solid support. By so doing a peptide array that represents epitope of the full length marker protein is created for each marker protein. The body sample is then contacted with this peptide array and antibodies that have been raised by the immune system of the individual against (an epitope) of one of the markers proteins will bind the respective immobilized fragment of the peptide array (cf. again Example 6). Any suitable fragment of any of EBNA1, EBNA3, EBNA4, EBNA6, BZLF1, LMP1 and VP26 can be used in such a solid phase peptide array. As indicated in Table 1 below (see SEQ ID NOs: 1-21 in Table 1), the full-length marker protein and/or the peptide fragment can be derived from any EBV virus strain, including but not limited to the strains AG876, B95-8, GD-1, Cao, or Raji, to name only a few illustrative known strains. Specific fragments that are used in exemplary embodiments of the inventions are provided in SEQ ID Nos.: 22-135, SEQ ID NOs: 149-151 and SEQ ID NOs: 153-155. It is of course possible to use any other suitable fragments of the marker protein sequences of SEQ ID Nos.: 1-21 as defined below. It is within the knowledge of the skilled person to select suitable fragments of the marker proteins (see again Example 6). Any other protein/peptide based technique, such as Luminex technology, or flow cytometry using biotin-labeled proteins or peptides to detect antibody-bearing memory B cells can also be used to detect antibodies that bind to the peptide array. Likewise the absence of a marker can also refer to the absence of an indirect measurement to the marker, such as the absence of an immune response to a fragment of the marker.

In the context of the present invention, the term “marker” refers to a protein found in a sample from the body of an individual that is characteristic of a certain condition or an immune response directed towards this protein or a fragment thereof. Thus a “marker characteristic of Epstein-Barr virus infection” is a protein that is known to be expressed in either acute or latent Epstein-Barr syndrome and that can therefore be identified in a sample from the body of an individual suffering from Epstein-Barr syndrome. The markers of particular interest of the present invention are EBNA3, EBNA4, EBNA6, EBNA1, LMP-1, BZLF1 and VP26, having the respective protein sequences of SEQ ID NOs.:1-21. Most preferably, the markers of the present invention are selected from EBNA1, EBNA4 and EBNA6. These markers can have an amino acid sequence corresponding to any one of SEQ ID NOs.:4, 5, 6, 10, 11, 12, 13, 14 or 15. The identity of these sequences is summarized in Table 1 below.

In the context of the invention, a “protein fragment” is a fragment of one of the markers of the invention mentioned above. The term “protein fragment” could be replaced with the term “peptide” or “polypeptide fragment”. A protein fragment according to the invention can be any fragment of any one of SEQ ID NOs.: 1-21. In a preferred embodiment, the protein fragment is between 4 and 70 amino acids, between 4 and 25 amino acids or between 4 and 15 amino acids in length from the specified regions of the following sequences: amino acid 80-105 of SEQ ID NO.: 1; amino acid 158-245 of SEQ ID NO.: 1; amino acid 80-105 of SEQ ID NO.: 2; amino acid 158-245 of SEQ ID NO.: 2; amino acid 80-105 of SEQ ID NO.: 3; amino acid 158-245 of SEQ ID NO.: 3; amino acid 479-555 of SEQ ID NO.: 4; amino acid 394-414 of SEQ ID NO: 5, amino acid 479-555 of SEQ ID NO.: 5; amino acid 479-555 of SEQ ID NO.: 6; amino acid 1-30 of SEQ ID NO.: 7; amino acid 76-110 of SEQ ID NO.: 7; amino acid 154-431 of SEQ ID NO.: 7; amino acid 489-548 of SEQ ID NO.: 7; amino acid 784-828 of SEQ ID NO.: 7; amino acid 1-30 of SEQ ID NO.: 8; amino acid 77-111 of SEQ ID NO.: 8; amino acid 155-432 of SEQ ID NO.: 8; amino acid 503-567 of SEQ ID NO.: 8; amino acid 803-828 of SEQ ID NO.: 8; amino acid 1-30 of SEQ ID NO.: 9; amino acid 77-111 of SEQ ID NO.: 9; amino acid 155-432 of SEQ ID NO.: 9; amino acid 503-567 of SEQ ID NO.: 9; amino acid 794-819 of SEQ ID NO.: 9; amino acid 161-204 of SEQ ID NO.: 10; amino acid 230-259 of SEQ ID NO.: 10; amino acid 290-351 of SEQ ID NO.: 10; amino acid 383-450 of SEQ ID NO.: 10; amino acid 524-549 of SEQ ID NO.: 10; amino acid 614-719 of SEQ ID NO.: 10; amino acid 790-815 of SEQ ID NO.: 10; amino acid 922-946 of SEQ ID NO.: 10; amino acid 161-204 of SEQ ID NO.: 11; amino acid 230-259 of SEQ ID NO.: 11; amino acid 290-351 of SEQ ID NO.: 11; amino acid 383-450 of SEQ ID NO.: 11; amino acid 524-549 of SEQ ID NO.: 11; amino acid 614-711 of SEQ ID NO.: 11; amino acid 782-807 of SEQ ID NO.: 11; amino acid 914-938 of SEQ ID NO.: 11; amino acid 161-204 of SEQ ID NO.: 12; amino acid 230-259 of SEQ ID NO.: 12; amino acid 290-351 of SEQ ID NO.: 12; amino acid 383-450 of SEQ ID NO.: 12; amino acid 524-549 of SEQ ID NO.: 12; amino acid 614-711 of SEQ ID NO.: 12; amino acid 782-807 of SEQ ID NO.: 12; amino acid 914-938 of SEQ ID NO.: 12; amino acid 53-204 of SEQ ID NO.: 13; amino acid 236-306 of SEQ ID NO.: 13; amino acid 519-575 of SEQ ID NO.: 13; amino acid 632-696 of SEQ ID NO.: 13; amino acid 855-980 of SEQ ID NO.: 13; amino acid 53-204 of SEQ ID NO.: 14; amino acid 236-306 of SEQ ID NO.: 14; amino acid 518-543 of SEQ ID NO.: 14; amino acid 605-669 of SEQ ID NO.: 14; amino acid 785-903 of SEQ ID NO.: 14; amino acid 741-779 of SEQ ID NO: 14 amino acid 53-204 of SEQ ID NO.: 15; amino acid 236-306 of SEQ ID NO.: 15; amino acid 518-543 of SEQ ID NO.: 15; amino acid 570-634 of SEQ ID NO.: 15; amino acid 802-920 of SEQ ID NO.: 15; amino acid 26-51 of SEQ ID NO.: 16; amino acid 122-147 of SEQ ID NO.: 16; amino acid 173-204 of SEQ ID NO.: 16; amino acid 26-51 of SEQ ID NO.: 17; amino acid 122-147 of SEQ ID NO.: 17; amino acid 173-204 of SEQ ID NO.: 17; amino acid 26-51 of SEQ ID NO.: 18; amino acid 122-147 of SEQ ID NO.: 18; amino acid 173-204 of SEQ ID NO.: 18; amino acid 26-51 of SEQ ID NO.: 19; amino acid 122-147 of SEQ ID NO.: 19; amino acid 173-204 of SEQ ID NO.: 19; amino acid 26-51 of SEQ ID NO.: 20; amino acid 122-147 of SEQ ID NO.: 20; amino acid 173-204 of SEQ ID NO.: 20; amino acid 1-21 of SEQ ID NO.: 21; or amino acid 50-99 of SEQ ID NO.: 21.

In one embodiment, the fragments can be 5, 6, 7, 8, 9, 10, 11, 12, 13, 14, 15, 16, 17, 18, 19, 20, 21, 22, 23, 24, or 25 amino acids in length. In a preferred embodiment, the fragments are 15 amino acids. In a further preferred embodiment, the fragments correspond to one or more of SEQ ID NOs.: 22-135, of SEQ ID NO: 149-151 or of SEQ ID NO: 153-155. The above fragments can of course be part of a larger construct or vector and may have various additional modifications known in the art such as linkers or labels. The sequences referred to above are summarized in Table 1 below.

| | |
|---|---|
| SEQ ID NO: 1 | Trans-activator protein BZLF1 of |
| | Epstein Barr Virus Strain AG876 |
| | (UniProtKB accession number Q1HVG1 |
| SEQ ID NO: 2 | Trans-activator protein BZLF1 of |
| | Epstein Barr Virus Strain B95-8 |
| | (UniProtKB accession number P03206) |
| | (BZLF1_EBVB9) |
| SEQ ID NO: 3 | Trans-activator protein BZLF1 of |
| | Epstein Barr Virus Strain GD1 |
| | (UniProtKB accession number Q3KSS8) |
| SEQ ID NO: 4 | Epstein-Barr nuclear antigen 1 |
| | (EBNA1) of Strain AG876, |
| | UniProtKB accession number Q1HVF7 |
| SEQ ID NO: 5 | EBNA1 of Strain B95-8, UniProtKB |
| | accession number |
| | P03211 (EBNA1_EBVB9) |
| SEQ ID NO: 6 | EBNA1 of Strain GD1 (UniProtKB |
| | accession number Q3KSS4) |
| SEQ ID NO: 7 | Epstein-Barr nuclear antigen 3 |
| | (EBNA3) of Strain AG876, |
| | UniProtKB accession number Q69138) |
| SEQ ID NO: 8 | EBNA3 of Strain B95-8 (UniProtKB |
| | accession number |
| | P12977 (EBNA3_EBVB9) |
| SEQ ID NO: 9 | EBNA3 of Strain GD1 (UniProtKB |
| | accession number Q3KST2) |
| SEQ ID NO: 10 | Epstein-Barr nuclear antigen 4 |
| | (EBNA4) of Strain AG876; |
| | (UniProtKB accession number Q1HVG4) |
| SEQ ID NO: 11 | EBNA4 of Strain B95-8 |
| | UniProtKB accession number |
| | P03203 (EBNA4_EBVB9) |
| SEQ ID NO: 12 | EBNA4 Strain of GD1 UniProtKB |
| | accession number Q3KST1 |
| SEQ ID NO: 13 | Epstein-Barr nuclear antigen |
| | (EBNA6) of Strain AG876 |
| | UniProtKB accession number Q69140) |
| SEQ ID NO: 14 | EBNA6 of Strain B95-8 UniProtKB |
| | accession number |
| | P03204 (EBNA6_EBVB9) |
| SEQ ID NO: 15 | EBNA6 of Strain GD1 UniProtKB |
| | accession number |
| SEQ ID NO: 16 | Latent membrane protein 1 (LMP1) |
| | of Strain AG876 UniProtKB |
| | accession number Q1HVB3 |
| | (LMP1_EBVA8) |
| SEQ ID NO: 17 | LMP1 of Strain B95-8 UniProtKB |
| | accession number P03230 |
| | (LMP1_EBVB9) |
| SEQ ID NO: 18 | LMP1 of Strain Cao UniProtKB |
| | accession number P29362 |
| | (LMP1_EBVC) |
| SEQ ID NO: 19 | LMP1 of Strain GD1 UniProtKB |
| | accession number P00741 |
| | (LMP1_EBVG) |
| SEQ ID NO: 20 | LMP1 of Strain Raji UniProtKB |
| | accession number P13198 |
| | (LMP1_EBVR) |
| SEQ ID NO: 21 | VP26 of Strain B95-8 UniProtKB |
| | accession number P14348 |
| | (VP26_EBVB9) |
| SEQ ID NO: 22 | BZLF1_EBVB9_0085: AYQAYAAPQLFPVSD |
| SEQ ID NO: 23 | BZLF1_EBVB9_0163: QQPESLEECDSELEI |
| SEQ ID NO: 24 | BZLF1_EBVB9_0169: EECDSELEIKRYKNR |
| SEQ ID NO: 25 | BZLF1_EBVB9_0172: DSELEIKRYKNRVAS |
| SEQ ID NO: 26 | BZLF1_EBVB9_0193: FKQLLQHYREVAAAK |
| SEQ ID NO: 27 | BZLF1_EBVB9_0196: LLQHYREVAAAKSSE |
| SEQ ID NO: 28 | BZLF1_EBVB9_0223: PSLDVDSIIPRTPDV |
| SEQ ID NO: 29 | BZLF1_EBVB9_0226: DVDSIIPRTPDVLHE |
| SEQ ID NO: 30 | EBNA1_EBVB9_0484: GLRALLARSHVERTT |
| SEQ ID NO: 31 | EBNA1_EBVB9_0514: KTSLYNLRRGTALAI |
| SEQ ID NO: 32 | EBNA1_EBVB9_0517: LYNLRRGTALAIPQC |
| SEQ ID NO: 33 | EBNA1_EBVB9_0523: GTALAIPQCRLTPLS |
| SEQ ID NO: 34 | EBNA1_EBVB9_0526: LAIPQCRLTPLSRLP |
| SEQ ID NO: 35 | EBNA1_EBVB9_0532: RLTPLSRLPFGMAPG |
| SEQ ID NO: 36 | EBNA1_EBVB9_0535: PLSRLPFGMAPGPGP |
| SEQ ID NO: 37 | EBNA3_EBVB9_0004: DRPGPPALDDNMEEE |
| SEQ ID NO: 38 | EBNA3_EBVB9_0010: ALDDNMEEEVPSTSV |
| SEQ ID NO: 39 | EBNA3_EBVB9_0082: PPQPDLPGREAILRR |
| SEQ ID NO: 40 | EBNA3_EBVB9_0088: PGREAILRRFPLDLR |
| SEQ ID NO: 41 | EBNA3_EBVB9_0091: EAILRRFPLDLRTLL |
| SEQ ID NO: 42 | EBNA3_EBVB9_0160: KWRLQTLAAGWPMGY |
| SEQ ID NO: 43 | EBNA3_EBVB9_0163: LQTLAAGWPMGYQAY |
| SEQ ID NO: 44 | EBNA3_EBVB9_0184: YTDHQTTPTFVHLQA |
| SEQ ID NO: 45 | EBNA3_EBVB9_0196: LQATLGCTGGRRCHV |
| SEQ ID NO: 46 | EBNA3_EBVB9_0199: TLGCTGGRRCHVTFS |
| SEQ ID NO: 47 | EBNA3_EBVB9_0211: TFSAGTFKLPRCTPG |
| SEQ ID NO: 48 | EBNA3_EBVB9_0220: PRCTPGDRQWLYVQS |
| SEQ ID NO: 49 | EBNA3_EBVB9_0244: NPRYSIFFDYMAIHR |
| SEQ ID NO: 50 | EBNA3_EBVB9_0247: YSIFFDYMAIHRSLT |
| SEQ ID NO: 51 | EBNA3_EBVB9_0250: FFDYMAIHRSLTKIW |
| SEQ ID NO: 52 | EBNA3_EBVB9_0280: LGFLQRTDLSYIKSF |
| SEQ ID NO: 53 | EBNA3_EBVB9_0283: LQRTDLSYIKSFVSD |
| SEQ ID NO: 54 | EBNA3_EBVB9_0319: QAWNAGFLRGRAYGI |
| SEQ ID NO: 55 | EBNA3_EBVB9_0325: FLRGRAYGIDLLRTE |
| SEQ ID NO: 56 | EBNA3_EBVB9_0331: YGIDLLRTEGEHVEG |
| SEQ ID NO: 57 | EBNA3_EBVB9_0370: VSRGGPKVKRPPIFI |
| SEQ ID NO: 58 | EBNA3_EBVB9_0373: GGPKVKRPPIFIRRL |
| SEQ ID NO: 59 | EBNA3_EBVB9_0412: STYGTPRPPVPKPRP |
| SEQ ID NO: 60 | EBNA3_EBVB9_0508: PIVRPWEPSLTQAAG |
| SEQ ID NO: 61 | EBNA3_EBVB9_0544: ALERPVYPKPVRPAP |
| SEQ ID NO: 62 | EBNA3_EBVB9_0547: RPVYPKPVRPAPPKI |
| SEQ ID NO: 63 | EBNA3_EBVB9_0808: LGYTLHGLNHPGVPV |
| SEQ ID NO: 64 | EBNA4_EBVB9_0166: RWKLLSSCRSWRMGY |
| SEQ ID NO: 65 | EBNA4_EBVB9_0169: LLSSCRSWRMGYRTH |
| SEQ ID NO: 66 | EBNA4_EBVB9_0178: MGYRTHNLKVNSFES |
| SEQ ID NO: 67 | EBNA4_EBVB9_0184: NLKVNSFESGGDNVH |
| SEQ ID NO: 68 | EBNA4_EBVB9_0235: IETAFLMARRARSLS |
| SEQ ID NO: 69 | EBNA4_EBVB9_0238: AFLMARRARSLSAER |
| SEQ ID NO: 70 | EBNA4_EBVB9_0295: HIREWFRQCTGRPKA |
| SEQ ID NO: 71 | EBNA4_EBVB9_0307: PKAAKPWLRAHPVAI |
| SEQ ID NO: 72 | EBNA4_EBVB9_0310: AKPWLRAHPVAIPYD |
| SEQ ID NO: 73 | EBNA4_EBVB9_0331: EIDLAYARGQAMNIE |
| SEQ ID NO: 74 | EBNA4_EBVB9_0388: LPYNPTVYGRPAVFD |
| SEQ ID NO: 75 | EBNA4_EBVB9_0394: VYGRPAVFDRKSDAK |
| SEQ ID NO: 76 | EBNA4_EBVB9_0430: HRKKKAARTEQPRAT |
| SEQ ID NO: 77 | EBNA4_EBVB9_0529: PQQPRAGRRGPCVFT |
| SEQ ID NO: 78 | EBNA4_EBVB9_0619: AAPRQWPMPLRPIPM |
| SEQ ID NO: 79 | EBNA4_EBVB9_0622: RQWPMPLRPIPMRPL |
| SEQ ID NO: 80 | EBNA4_EBVB9_0628: LRPIPMRPLRMQPIP |
| SEQ ID NO: 81 | EBNA4_EBVB9_0631: IPMRPLRMQPIPFNH |
| SEQ ID NO: 82 | EBNA4_EBVB9_0634: RPLRMQPIPFNHPVG |
| SEQ ID NO: 83 | EBNA4_EBVB9_0664: PTWAQIGHIPYQPTP |
| SEQ ID NO: 84 | EBNA4_EBVB9_0691: PATMQTPPRAPTPMS |
| SEQ ID NO: 85 | EBNA4_EBVB9_787: LKLQAALERQAAAGW |
| SEQ ID NO: 86 | EBNA4_EBVB9_0919: LGLGDIAVSSPSSSE |
| SEQ ID NO: 87 | EBNA6_EBVB9_0058: WGQSRGDENRGWMQR |
| SEQ ID NO: 88 | EBNA6_EBVB9_0064: DENRGWMQRIRRRRR |
| SEQ ID NO: 89 | EBNA6_EBVB9_0073: IRRRRRRRAALSGHL |
| SEQ ID NO: 90 | EBNA6_EBVB9_0076: RRRRRAALSGHLLDT |
| SEQ ID NO: 91 | EBNA6_EBVB9_0088: LDTEDNVPPWLPPHD |
| SEQ ID NO: 92 | EBNA6_EBVB9_0097: WLPPHDITPYTARNI |
| SEQ ID NO: 93 | EBNA6_EBVB9_0103: ITPYTARNIRDAACR |
| SEQ ID NO: 94 | EBNA6_EBVB9_0109: RNIRDAACRAVKQSH |
| SEQ ID NO: 95 | EBNA6_EBVB9_0145: VMAARQRLQDIRRGP |
| SEQ ID NO: 96 | EBNA6_EBVB9_0160: LVAEGGVGWRHWLLT |
| SEQ ID NO: 97 | EBNA6_EBVB9_0163: EGGVGWRHWLLTSPS |
| SEQ ID NO: 98 | EBNA6_EBVB9_0169: RHWLLTSPSQSWPMG |
| SEQ ID NO: 99 | EBNA6_EBVB9_0178: QSWPMGYRTATLRTL |
| SEQ ID NO: 100 | EBNA6_EBVB9_0181: PMGYRTATLRTLTPV |
| SEQ ID NO: 101 | EBNA6_EBVB9_0184: YRTATLRTLTPVPNR |
| SEQ ID NO: 102 | EBNA6_EBVB9_0241: AREAEVRFLRGKWQR |
| SEQ ID NO: 103 | EBNA6_EBVB9_0271: HHIWQNLLQTEENLL |
| SEQ ID NO: 104 | EBNA6_EBVB9_0277: LLQTEENLLDFVRFM |
| SEQ ID NO: 105 | EBNA6_EBVB9_0280: TEENLLDFVRFMGVM |
| SEQ ID NO: 106 | EBNA6_EBVB9_0283: NLLDFVRFMGVMSSC |
| SEQ ID NO: 107 | EBNA6_EBVB9_0286: DFVRFMGVMSSCNNP |
| SEQ ID NO: 108 | EBNA6_EBVB9_0295: SSCNNPAVNYWFHKT |
| SEQ ID NO: 109 | EBNA6_EBVB9_0301: AVNYWFHKTIGNFKP |
| SEQ ID NO: 110 | EBNA6_EBVB9_0304: YWFHKTIGNFKPYYP |
| SEQ ID NO: 111 | EBNA6_EBVB9_0307: HKTIGNFKPYYPWNA |
| SEQ ID NO: 112 | EBNA6_EBVB9_0313: FKPYYPWNAPPNENP |
| SEQ ID NO: 113 | EBNA6_EBVB9_0331: RRGIKEHVIQNAFRK |
| SEQ ID NO: 114 | EBNA6_EBVB9_0334: IKEHVIQNAFRKAQI |
| SEQ ID NO: 115 | EBNA6_EBVB9_0340: QNAFRKAQIQGLSML |
| SEQ ID NO: 116 | EBNA6_EBVB9_0523: SSVSQPNKPHRKHQD |
| SEQ ID NO: 117 | EBNA6_EBVB9_0610: GPHIVTPPSARPRIM |
| SEQ ID NO: 118 | EBNA6_EBVB9_0625: APPVVRMFMRERQLP |
| SEQ ID NO: 119 | EBNA6_EBVB9_0631: MFMRERQLPQSTGRK |
| SEQ ID NO: 120 | EBNA6_EBVB9_0649: FWEMRAGREITQMQQ |
| SEQ ID NO: 121 | EBNA6_EBVB9_0790: PGYAGPWTPRSQHPC |
| SEQ ID NO: 122 | EBNA6_EBVB9_0796: WTPRSQHPCYRHPWA |
| SEQ ID NO: 123 | EBNA6_EBVB9_0799: RSQHPCYRHPWAPWS |
| SEQ ID NO: 124 | EBNA6_EBVB9_0850: FPHLQSETGPPRLQL |
| SEQ ID NO: 125 | EBNA6_EBVB9_0856: ETGPPRLQLSLVPLV |
| SEQ ID NO: 126 | EBNA6_EBVB9_0883: RAPIRPIPTRFPPPP |
| SEQ ID NO: 127 | LMP1_EBVB9_0031: LLLLALLFWLYIVMS |
| SEQ ID NO: 128 | LMP1_EBVB9_0127: LEMLWRLGATIWQLL |
| SEQ ID NO: 129 | LMP1_EBVB9_0178: LAILIWMYYHGQRHS |
| SEQ ID NO: 130 | LMP1_EBVB9_0181: LIWMYYHGQRHSDEH |
| SEQ ID NO: 131 | LMP1_EBVB9_0184: MYYHGQRHSDEHHHD |
| SEQ ID NO: 132 | VP26_EBVB9_0001: MARRLPKPTLQGRLE |
| SEQ ID NO: 133 | VP26_EBVB9_0055: FCYEEYVQRTFGVPR |
| SEQ ID NO: 134 | VP26_EBVB9_0064: TFGVPRRQRAIDKRQ |
| SEQ ID NO: 135 | VP26_EBVB9_0079: RASVAGAGAHAHLGG |
| SEQ ID NO: 148 | EBNA1_EBVB9_394-414: PPRRPPP |
| | GRRPFFHPVG EADYF |
| SEQ ID NO: 149 | EBNA1_EBVB9_0394: PPRRPPPGRRPFFHP |
| SEQ ID NO: 150 | EBNA1_EBVB9_0397: RPPPGRRPFFHPVGE |
| SEQ ID NO: 151 | EBNA1_EBVB9_0400: PGRRPFFHPVGEADY |
| SEQ ID NO: 152 | EBNA6_EBVB9_741-779: PPRRPPP |
| | GRRPFFHPVG EADYF |
| SEQ ID NO: 153 | EBNA6_EBVB9_0748: QGYQEPPAPQAPYQG |
| SEQ ID NO: 154 | EBNA6_EBVB9_0751: QEPPAPQAPYQGYQE |
| SEQ ID NO: 155 | EBNA6_EBVB9_0754: PAPQAPYQGYQEPPP |

A “latent Epstein-Barr virus (EBV) infection” is an EBV infection that is no longer acute. In latency, specific genes are expressed, in EBV these include EBERs and EBNAs, (see US patent application 2005/0074751 pages 4-5 for a discussion of EBV). According to this publication, in Type I latency, only EBNA-1 is expressed, in Type II latency, LMP-1, LMP-2A and LMP-2B are expressed and in Type III latency, LMP-1, LMP-2A, LMP-2B LP, EBNA-1, EBNA-2, EBNA-3A, EBNA-3B and EBNA-3C are expressed.

In the context of the present invention, a “sample obtained from the body of an individual” can be selected from the group consisting of a blood sample, a saliva sample, a urine sample, a nasopharygeal sample, a sample from a pharyngeal wash and a sample from a gastric or colonic biopsy.

The “physical disorder” to be differentiated from CFS refers to a physical disorder with very similar symptoms to CFS, such as depression or burn-out syndrome.

In the context of the present invention, an “immune response” is an antibody response found in a sample from an individual. The term immune response could also be replaced with the term antibody response or seroreactivity. The immune response can be measured by an immunoassay, which is in turn an assay that measures the immune response. Such an assay can be based on a peptide microarray or other systems where peptides are immobilized to solid phases. An immune response further is a T or B cell response.

The term “measuring the amount of a protein, a fragment thereof or the corresponding nucleotide” refers to standard methods known in the art such as immunostaining or quantative PCR.

It is noted that as used herein, the singular forms “a”, “an”, and “the”, include plural references unless the context clearly indicates otherwise. Thus, for example, reference to “a reagent” includes one or more of such different reagents and reference to “the method” includes reference to equivalent steps and methods known to those of ordinary skill in the art that could be modified or substituted for the methods described herein.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the present invention.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or sometimes when used herein with the term “having”.

When used herein “consisting of” excludes any element, step, or ingredient not specified in the claim element. When used herein, “consisting essentially of” does not exclude materials or steps that do not materially affect the basic and novel characteristics of the claim.

As described herein, “preferred embodiment” means “preferred embodiment of the present invention”. Likewise, as described herein, “various embodiments” and “another embodiment” means “various embodiments of the present invention” and “another embodiment of the present invention”, respectively.

Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention.

### EXAMPLES

The following examples illustrate the invention. These examples should not be construed as to limit the scope of this invention. The examples are included for purposes of illustration and the present invention is limited only by the claims.

### Methods:

### Study Population and Specimen Collection

Patients who were diagnosed with CFS according to Fukuda criteria without evidence of other severe medical, neurological or psychiatric disease (Fukuda 1994) were recruited from the outpatient clinic for Adult Immunodeficiencies of the Department of Medical Immunology of the Charité University Medicine Berlin between 2007 and 2013. Informed consent was obtained from all patients and healthy control donors and the study was approved by the Institutional Ethics Committee.

### Blood Samples

Blood was obtained from CFS patients or healthy subjects. Peripheral blood mononuclear cells (PBMC) were isolated by density gradient centrifugation using Ficoll Hypaque and either cryopreserved for T-cell analysis or used in cell culture stimulation assay for memory B-cell analysis and B-cell separation. Serum was obtained separately from CFS patients, depression patients and healthy subjects and stored at −20° C. for serum antibody analysis.

### Pharyngeal Wash Samples

Patients gargle with 5 ml of water for 2 minutes.

### Example 1

### Enzyme-Linked Immunosorbent Assay (ELISA)

### Methods:

EBNA IgG, VCA IgG and VCA IgM were detected using an immuno chemiluminescence assay (CLIA, DiaSorin, S.p.A., Saluggia, Italy) according to the manufacture's instructions. An Enzyme Immunoassay was used to detect EBV EBNA-1 IgG in the Labor Berlin GmbH. Seronegative subjects and subjects with recent EBV-infection were excluded from the analysis.

### Results:

In this experiment, it was found that a subset of CFS patients shows abnormal EBV serology. We compared serum EBV-VCA IgG, IgM, and EBNA1 IgG from patients (n=63) and controls (n=57) (FIG. 1A). All but 1 patient was EBV-VCA IgG positive, who was excluded from this analysis. While we did not observe a difference between VCA-IgG titers, IgG antibodies against EBNA1 were undetectable (≦20 U/ml) in 12.7% of CFS patients in contrast to 3.5% of healthy controls (p=0.066, FIG. 1B). Further, VCA IgM was more frequent in patients with 17.5% compared to 3.5% in healthy controls (FIG. 1C, p=0.013). In contrast, titers of CMV IgG and IgM revealed no difference between CFS patients and healthy controls (FIGS. 1D and E). Elevated EBV-VCA IgM and lack of EBNA1 IgG were detected in different patient subsets accounting for 30% of CFS patients with an abnormal EBV serology.

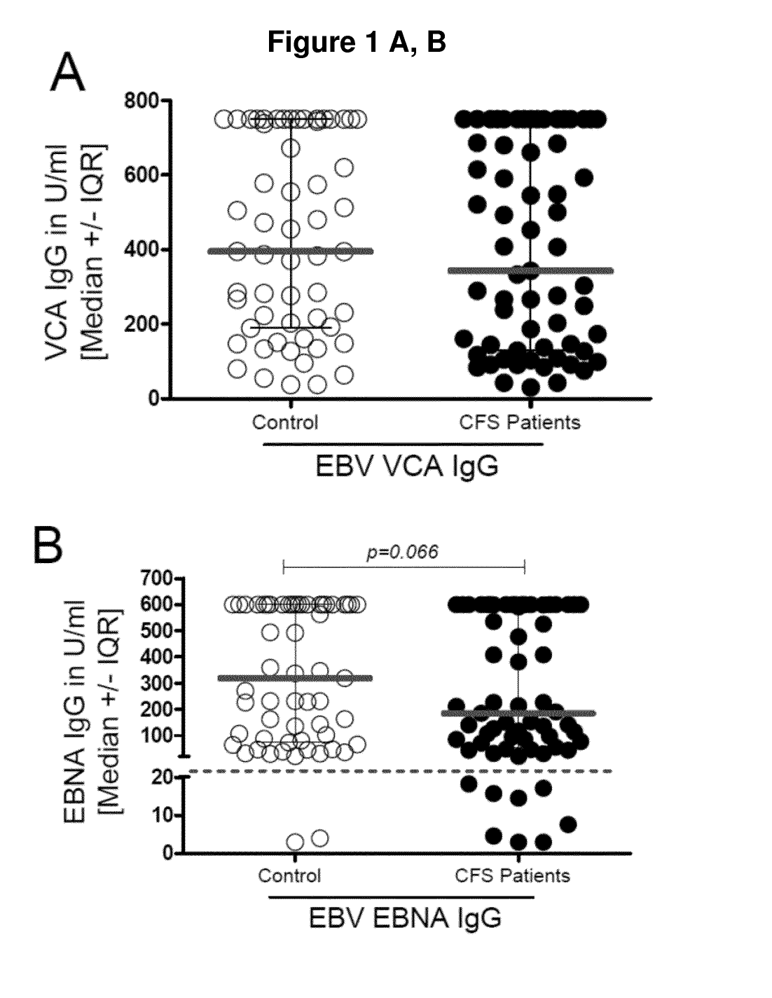

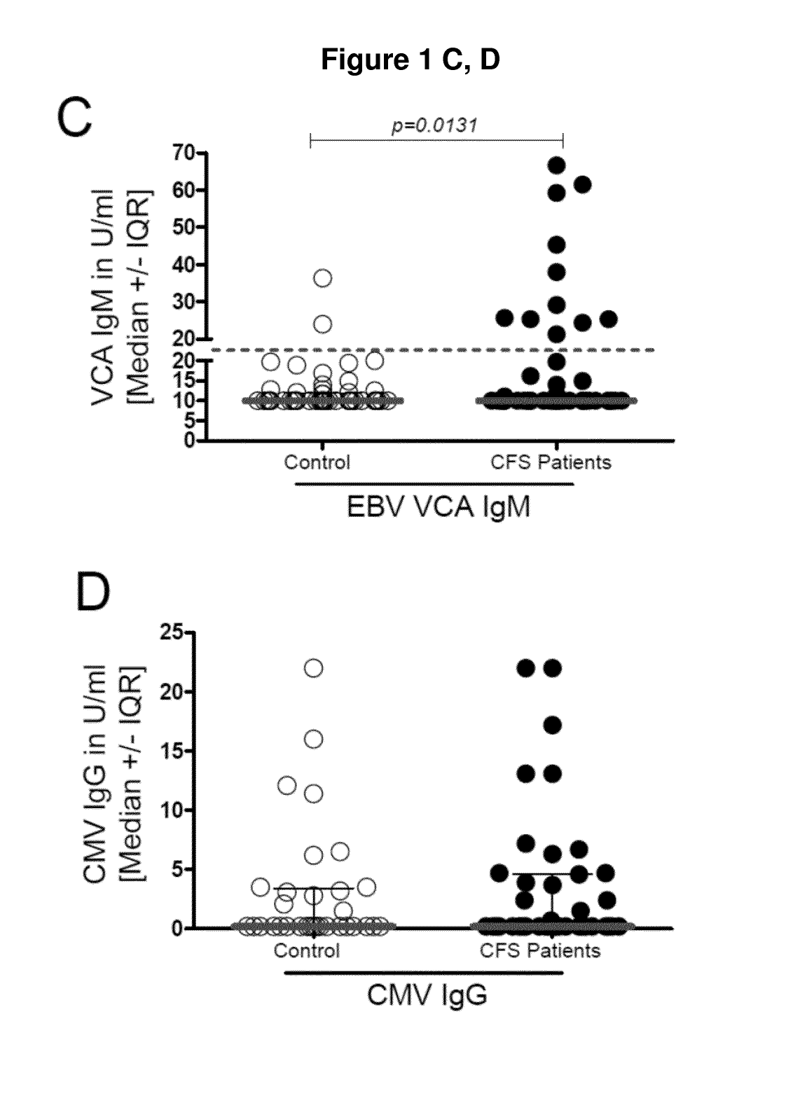

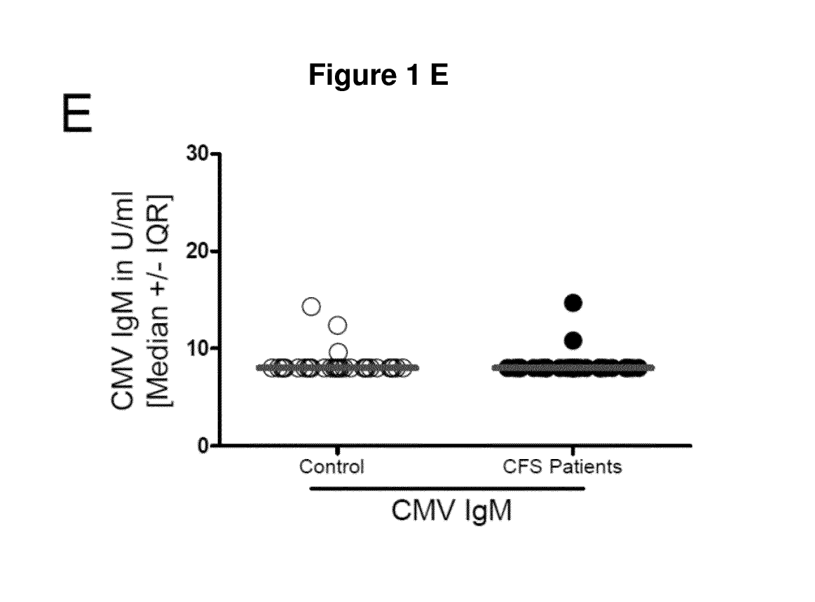

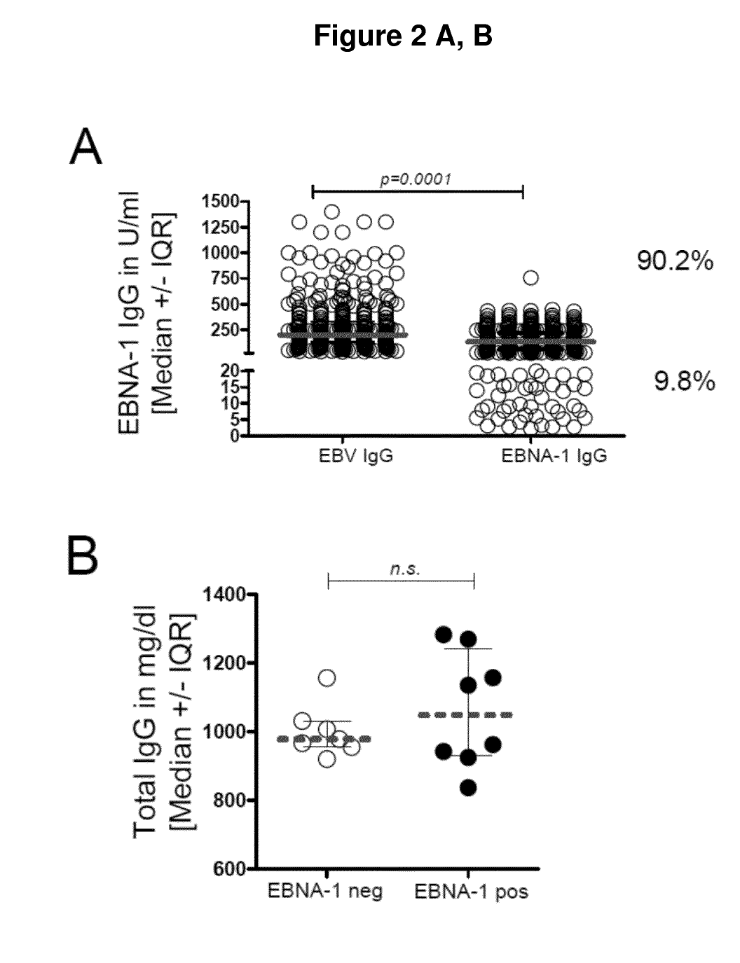

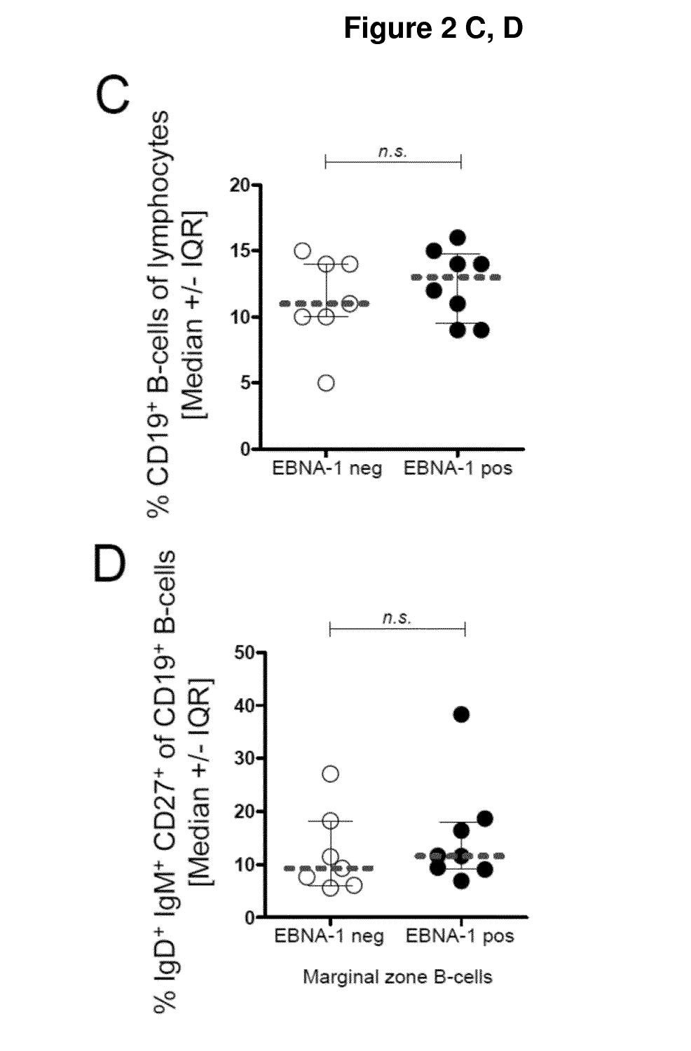

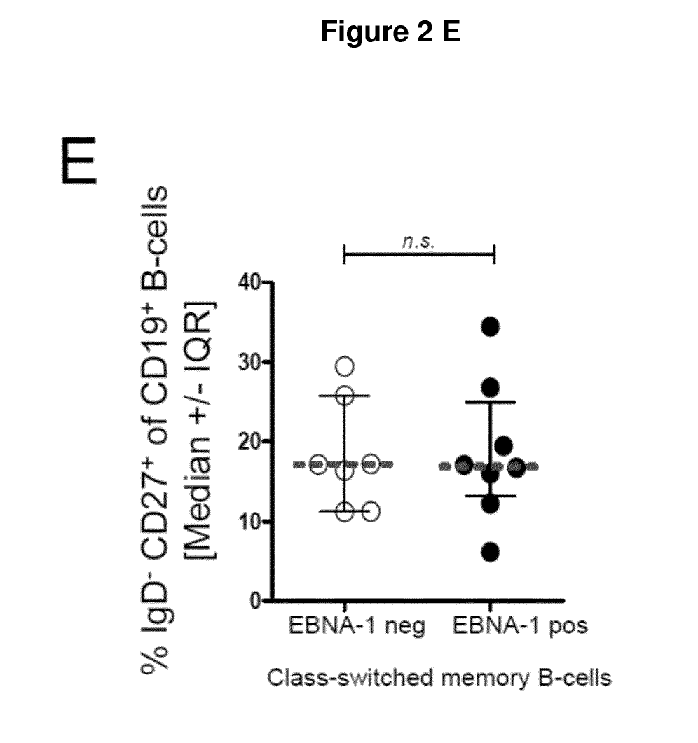

In a consecutive cohort of 397 CFS patients EBV-specific antibodies were measured with Enzyme Immunoassays determining IgG against a mixture of various EBV proteins or EBNA-1. Similarly, we observed a lack of EBNA-1 IgG in 9.8% of EBV-IgG positive patients (FIG. 2A). In a subset of EBNA-1 IgG positive and negative patients we further comparatively analyzed total IgG levels, frequencies of B-cells, and B-cell subsets. No difference in IgG as well as total and memory B-cell subset frequencies was seen in EBNA-1 IgG negative compared to EBNA-1 IgG positive CFS patients and numbers of B-cell subsets were within the normal range compared to the reference group of the Charité Immune diagnostic laboratory as indicated by the dashed line (FIG. 2B-E).

### Example 2

### Quantitative Real-Time PCR

### Methods:

Detection of EBV DNA in PBMCs was done by nested PCR for EBER-1 with the following primers forward 5′-TCC CGG GTA CAA GTC CCG-3′ (SEQ ID NO.: 136) and reverse 5′-TGA CCG AAG ACG GCA GAA AG-3′ (SEQ ID NO.: 137) at 900 nM. Detection has been performed with probe FAM-5′-TGG TGA GGA CGG TGT CTG TGG TTG TGT T-3′-TAMRA (SEQ ID NO.: 138) (Eurofins MWG Operon, Ebersberg Germany) at 5 pM. Amplification data were analyzed by an ABI PRISM 7700 Sequence Detection System (PE Applied Biosystems, California, USA). Successful DNA isolation was verified by histone replication with the primers forward 5′-CCA GAG CGC AGC TAT CGG T-3′ (SEQ ID NO.: 139) at 900 nM and reverse 5′-CAC GTT TGG CAT GGA TAG CAC-3′ (SEQ ID NO.: 140) at 50 nM and the probe FAM-5′-GCA AGT GAG GCC TAT CTG GTT GGC CTT T-3-TAMRA (SEQ ID NO.: 141) (Eurofins MWG Operon, Ebersberg Germany) at 5 pM. For EBNA-1 the following primers forward 5′-TACAGGACCTGGAAATGGCC-3′ (SEQ ID NO.: 142) and reverse 5′-TCTTTGAGGTCCACTGCCG-3′ (SEQ ID NO.: 143) at 15 pM were used. Detection has been performed with probe FAM-5′-AGGGAGACACATCTGGACCAGAAGGC-3′-TAMRA (SEQ ID NO.: 144) (Eurofins MWG Operon, Ebersberg Germany) at 10 pM. For BZLF-1 the following primers forward 5′-AAATTTAAGAGATCCTCGTGTAAAACATC-3′ (SEQ ID NO.: 145) and reverse 5′-CGCCTCCTGTTGAAGCAGAT-3′ (SEQ ID NO.: 146) at 30 pM were used. Detection has been performed with probe FAM-5′-ATAATGGAGTCAACATCCAGGCTTGGGC-3′-TAMRA (SEQ ID NO: 147) (Eurofins MWG Operon, Ebersberg Germany) at 10 pM. EBV copies/μg DNA were calculated as standard EBV copies/μl DNA divided by sample DNA concentration (μg/μl). Results above 35 copies/μg DNA were regarded as positive according to Holland et al. 1991, PNAS 88: 7276-7280).

### Results

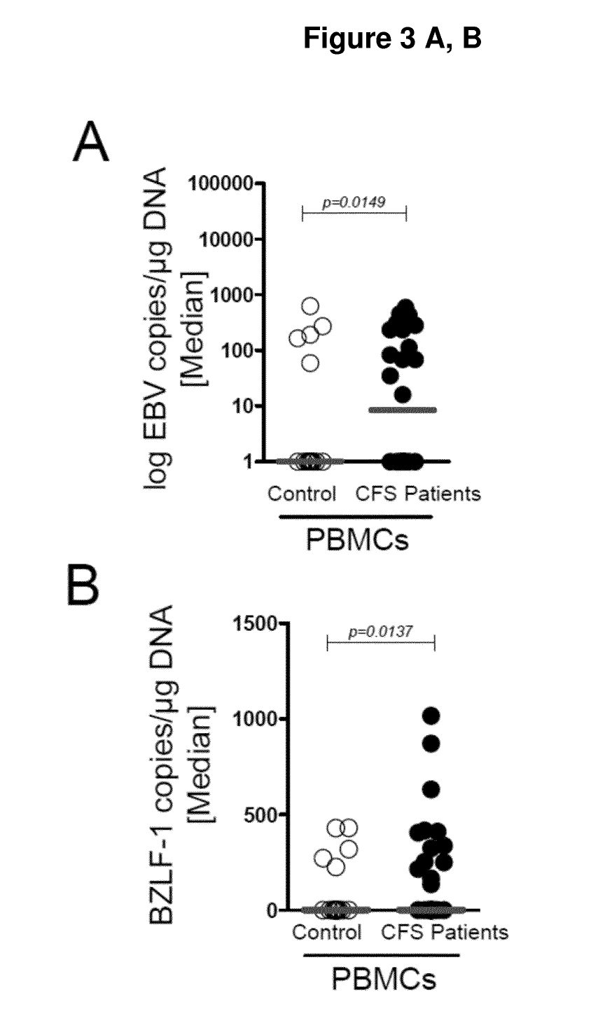

Real-time PCR was used to determine EBV viral load by EBER-1 gene expression in blood immune cells of patients with CFS and healthy controls. EBV-EBER-1 DNA copies were detected in 50% of patients (n=13/26) and in 17.2% (n=5/29) healthy controls in peripheral blood mononuclear immune cells (PBMC) (FIG. 3A). Further, EBV DNA was compared in pharyngeal washings of 10 CFS patients and 10 healthy controls revealing a positive EBER signal in 6 samples of both cohorts. Additionally, we tested 4 EBV seronegative CFS patients and detected no EBER-1 DNA in PBMCs (data not shown). We further tried to detect EBNA-1 copies in the DNA of EBER-1 positive CFS patients but only 2 of 6 patients were positive for EBNA-1. As a third gene we determined BZLF-1 copy numbers, revealing a signal in 13 of 27 CFS patients (48%) but only in 5 of 27 healthy controls (18%) (FIG. 3B). Interestingly no correlation between EBER-1 and BZLF-1 copies was seen in the patients and controls. Altogether EBV-EBER-1 or BZLF-1 copies could be detected in PBMC in 67% (n=18/27) of patients in contrast to 26% (n=7/27) of controls. To study lytic replication we further looked for EBV RNA but did not detect copies of BZLF-1 mRNA in either patients or controls, but low level transcripts of EBER-1 mRNA in most EBER-1 DNA positive patients but also in EBER1-DNA positive controls.

### Example 3

### B-Cell Memory Analysis by ELISpot-Assay

### Methods:

Analysis of memory B-cells was adapted from Crotty et al. (Crotty 2004, J. Immunol. Methods 286: 111-122). PBMCs were stimulated unspecifically with Pokeweed mitogen (PWM) at 10 ng/ml (Sigma Aldrich, Schnelldorf, Germany), Staphylococcus aureus Cowan at 1:10000 dilution (Merck, Darmstadt, Germany) and CpG at 6 μg/ml (InvivoGen, CA, USA) in RPMI 1640 (PAA Laboratories, Cölbe, Germany) supplemented with Penicillin/Streptomycin 100× (Biochrom, Berlin, Germany), L-Glutamine at 2 mM (Biochrom, Berlin, Germany) and β-Mercaptoethanol at 50 μM (Merck, Darmstadt, Germany) for 7 days at 37° C. in 5% CO₂. For T-cell independent stimulation B-cells from CFS patients were enriched with a RosetteSep CD3 depletion kit according to the manufacturer's instructions (Stemcell Technologies, Grenoble, France). 2.5·10⁶ B-cells/well were kept in 1 mL IMDM (PAA Laboratories, Cölbe, Germany) with 10% heat-inactivated AB serum (Valley Biomedical, Winchester, Va., USA), 5 μg/mL Insulin (Sigma Aldrich, Schnelldorf, Germany)/Transferrin (Sigma Aldrich, Schnelldorf, Germany), 5 ng/ml selenium (Sigma Aldrich, Schnelldorf, Germany), 1.25 μg/ml CpG (Invivogen, CA, USA), 300 U/ml IL-2 (Chiron-Behring, Liederbach, Germany), 12.5 ng/ml IL-10 (ImmunoTools, Friesoythe) and 500 ng/ml IL-21 (ImmunoTools, Friesoythe, Germany). Cells were cultured for 7 days at 37° C. in 5% CO₂. After stimulation, the cells were transferred at a concentration of 1×10⁶/100 μl into a 96-well multiscreen HTS-IP filter plate (Merck Millipore, MA, USA) coated with purified, recombinant EBV-VCA at 0.1 μg/well (tebu-bio, Le-Perray-en-Yvelines, France) and EBV-EBNA-1 at 1 μg/well (tebu-bio, Le-Perray-en-Yvelines, France) and purified EBV-lysate at 1:20 dilution (tebu-bio, Le-Perray-en-Yvelines, France). For the analysis of total IgG anti-human IgG Fc-fragment antibody (Jackson Immunoresearch, PA, USA) was coated at a concentration of 1.2 μg/well and cells were seeded at a concentration of 1.25×10⁴/100 μl, 6250/100 μl and 3125/100 μl for 6 h. Secreted IgGs were detected using an anti-human IgG, F(ab′)2 fragment coupled to Biotin at 1 μg/ml (Biosource, Life Technologies, Darmstadt, Germany) and Horseradish Peroxidase Avidin D at 5 ng/ml (Vector Laboratories, MI, USA). IgG spots were visualized by adding 3-Amino-9-ethylcarbazole (Sigma-Aldrich, Schnelldorf, Germany). Plates were scanned and spots enumerated on a CTL Immunoplate reader using Immunospot Academic software (Cellular Technology Ltd, OH, USA). Frequencies were expressed as the ratio of the mean number of antigen specific spots and mean number of total IgG spots.

### Results:

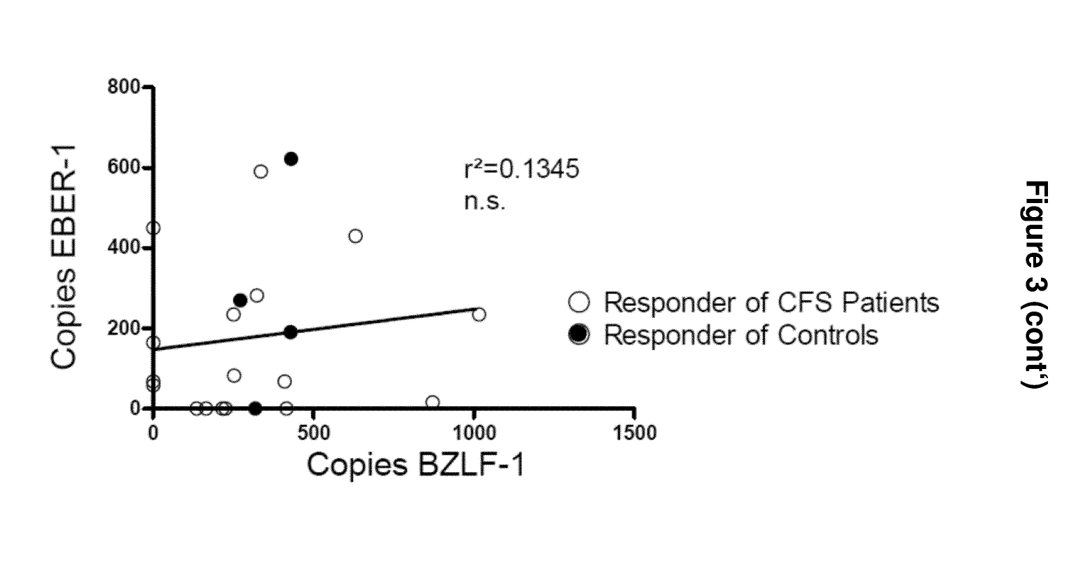

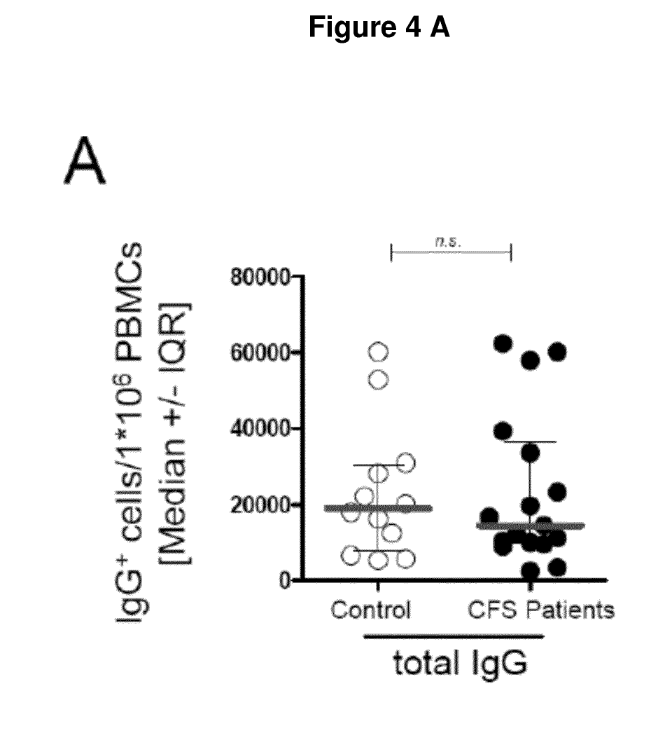

In this experiment, it was found that EBV specific memory B-cells are low or absent in most CFS patients. No difference between CFS patients and healthy controls was detected for total IgG producing B-cells quantified by ELISpot assay (FIG. 4A). However, the CFS patients had significantly reduced frequencies of B-cells producing antibodies against EBV lysate. In contrast we detected no difference in ASCs producing antibodies against HSV or CMV lysate analyzed in a subset of patients and controls (FIG. 4B). In addition using overlapping peptide pools, VCA-specific (FIG. 4C) and EBNA-1 IgG secreting B-cells were analyzed (FIG. 4D) which were low or absent in most CFS patients analyzed.

Taken together, the lack of memory B-cells despite the presence of normal antibody levels in many patients suggests a secondary immune deficiency or exhaustion of the EBV-specific B-cell response in patients with CFS.

### Example 4

### T Cell Response by Cytokine Analysis

### Methods:

Antigen specific T-cell response was measured by cytokine production in culture supernatants of PBMCs stimulated with either 1 μg/ml SEB (Sigma-Aldrich, Schnelldorf, Germany), 1 μg/ml EBV total lysate or 1 μg/ml of the EBV peptide EBNA-1 (JPT, Berlin, Germany) for 48 h. 2×10⁶ PBMCs were kept in 1 ml serumfree RPMI (PAA Laboratories, Cölbe, Germany) with 2% Hepes buffer, 1% L-Glutamin (Biochrom, Berlin, Deutschland) and 0.5% Gentamycin (Merck, Darmstadt, Germany). IFN-γ, IL-10, IL-2 and TNF-α were measured in cell culture supernatants with a 7-plex-Immunoassay (Merck Millipore, MA, USA) on a Luminex 200 (Luminex, TX, USA) according to manufacturer's instructions.

### Results:

In this experiment, it was found that CFS patients show diminished T-cell cytokine response against EBV. First, EBV lysate induced production of several cytokines that were analyzed in whole blood revealed a reduced number of IFN-γ responders in the patient group with 50% (n=11/22) vs. 69% (n=20/29) in the control group (FIG. 5A). Using overlapping 15-mer peptides from various EBV proteins for stimulation only few responders could be detected in healthy controls as shown for EBNA-1 induced IFN-γ. Similar IFN-γ levels were observed in response to the T-cell super-antigen SEB in patients and controls. In addition, significantly reduced levels of TNF-α and a lower number of patients producing IL-2 were observed, while the IL-10 response was not diminished in patients in response to EBV lysate (FIG. 5B).

### Example 5

### T-Cell Response Assessed by Flow Cytometry after In Vitro Expansion

### Methods:

EBV specific memory T-cells were analyzed after stimulation with EBNA-1 peptides and expansion in vitro as recently described (Guerreiro 2010). After overnight incubation of PBMCs in IMDM (PAA Laboratories, Cölbe, Germany) containing 10% AB serum (Valley Biomedical, Winchester, Va., USA) at 37° C. in 5% CO₂, cells were stimulated with 1 μg/mL EBNA-1 peptide pool (JPT, Berlin, Germany), 50 IU/mL rhIL-2 (Chiron-Behring, Liederbach, Germany) and 10 ng/mL rhIL-7 (ImmunoTools, Friesoythe, Germany) in 96-well round bottom plates at a concentration of 2×10⁵ cells per well. On day 3, 5 and 7 media and IL-2 50 ng/μl were renewed. IL-7 5 ng/μl was added on day 7 of culture, cells were harvested, washed and stained for cytokines.

### Multiparameter Flow Cytometry

Intracellular and extracellular staining was applied for T-cell analysis after 10 days of expansion. 2×10⁶ PBMCs were restimulated with EBNA-1 peptide pool (JPT, Berlin, Germany) (1 μg/mL) or DMSO (1 μg/mL) (Sigma Aldrich, Schnelldorf, Germany) as negative control for 5 h. Brefeldin A (7.5 μg/mL) (Sigma Aldrich, Schnelldorf, Germany) was added after 1 h of stimulation. Live/dead cells were discriminated using an amine reactive dye (Invitrogen, Life Technologies, Darmstadt, Germany) and stained with fluorescence conjugated monoclonal antibody against CD3, CD4, CD8 and IFN-γ, TNF-α and IL-2 (BD Biosciences, NJ, USA). Background events in DMSO controls were subtracted from events counted in response to EBNA-1 stimulation. Data acquisition was performed on BD LSR II (Becton Dickinson, NJ, USA) and analysis was done using FlowJo software.

### Statistical Analysis

Statistical data analyses were done using the software SPSS Statistics 19 and GraphPad Prism 5. Nonparametric statistical methods were used. Continuous variables were expressed as median and interquartile range (IQR), if not indicated otherwise. Univariate comparisons of two independent groups were done using the Mann-Whitney-U test. For association analysis Fisher's exact test was used. A p-value of <0.05 was considered statistically significant.

### Results:

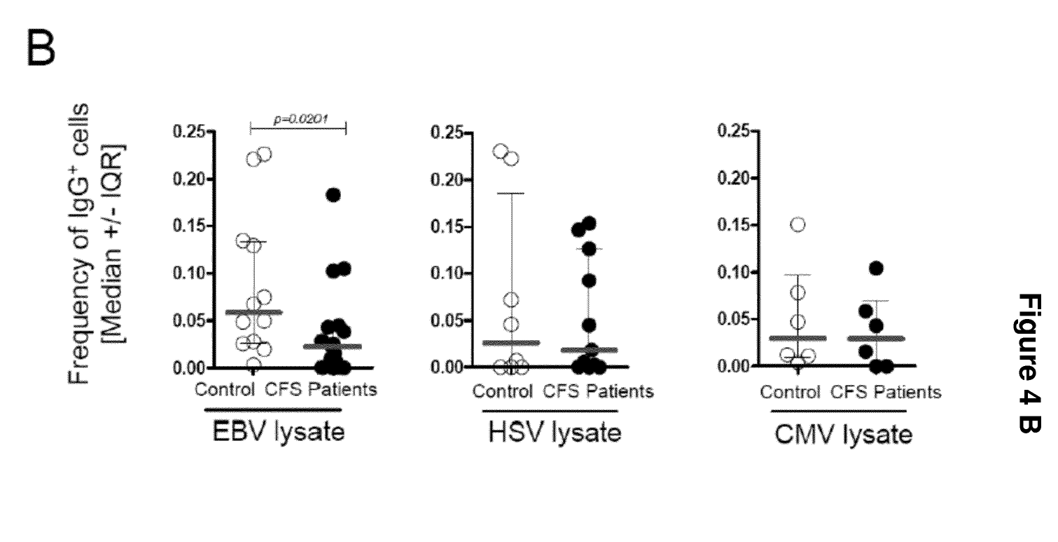

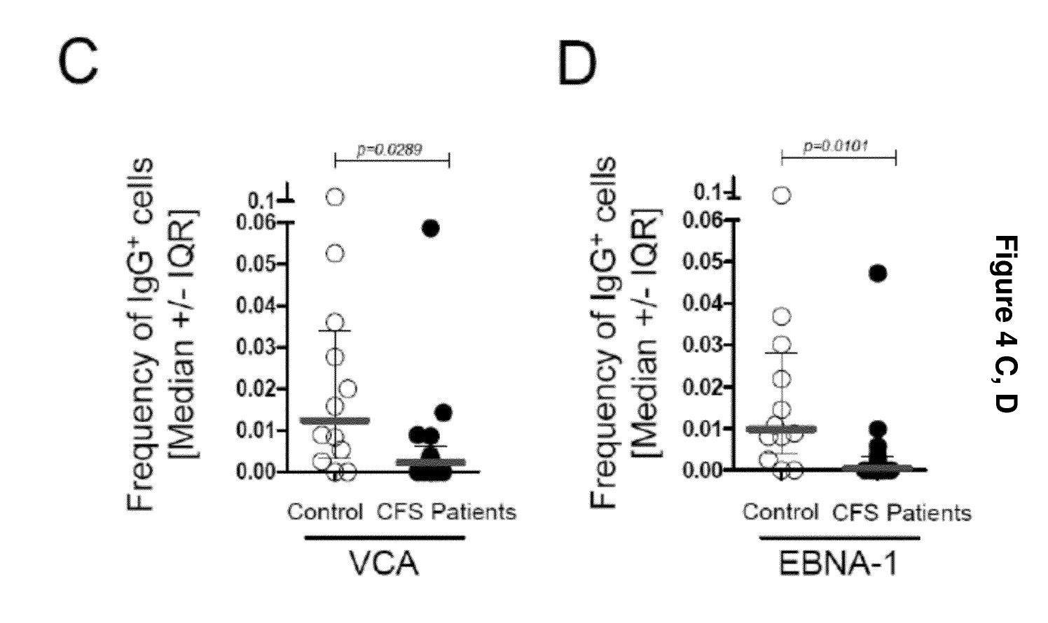

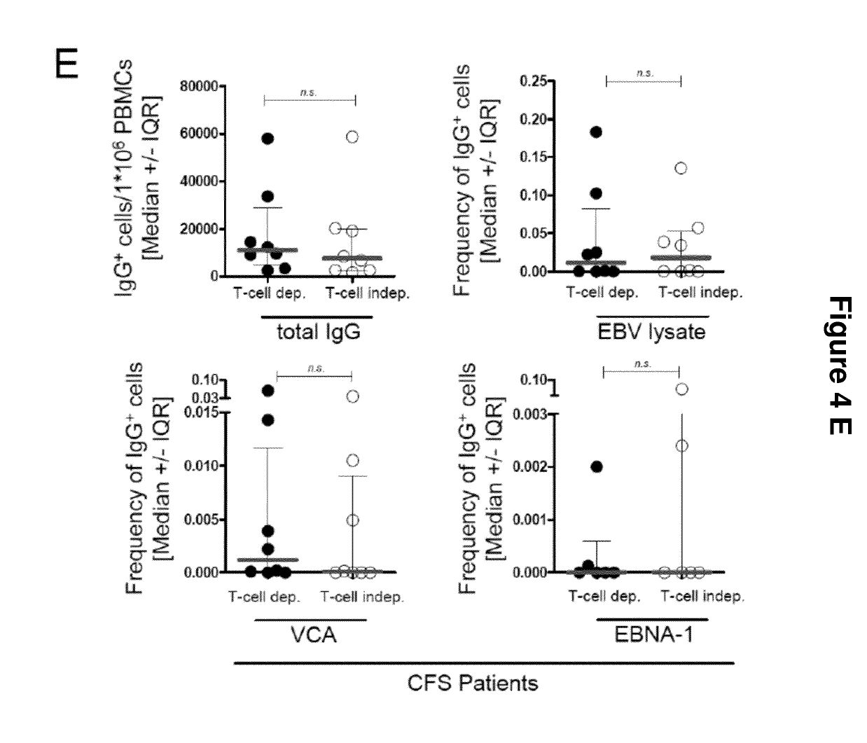

In this experiment, it was found that CFS patients show reduced EBV specific multifunctional memory T-cells. Interferon (IFN)-γ⁺ Tumor necrosis factor (TNF)-α⁺ and Interleukine (IL)-2⁺ producing CD3⁺ CD8⁺ and CD3⁺ CD4⁺ T-cells were analyzed by flow cytometry. While we observed similar frequencies of single producers in CFS patients and the control group frequencies of IFN-γ⁺ TNF-α⁺ double producing CD4⁺ T-cells and of IFN-γ⁺ TNF-α⁺ IL-2⁺ triple producing so called multifunctional CD8⁺ and CD4⁺ T-cells were significantly lower in the patient group (FIG. 6A-C). Frequencies of IFN-γ⁺ IL-2⁺ and TNF-α⁺ IL-2⁺ double positive T-cells were too low to be reliably determined.

### Example 6

### Screening of Sera from Patients with CFS, Depression and Healthy Donors

This experiment follows the observation that in CFS patients altered titers of EBV specific antibodies are observed. Peptide microarray experiments with sera from 36 CFS patients, 34 healthy volunteers and 20 depression patients show a skewed antibody repertoire against EBV-derived antigens with different responses against various peptides from EBNA1, EBNA3, EBNA4, EBNA6, BZLF1, LMP1, and VP26.

### Methods:

A library of 1465 peptides representing a 15/11 scan through EBV-antigens BLRF2, BZLF1, EBNA1, EBNA3, EBNA4, EBNA6, LMP1 and VP26 was applied to microarrays. The preparation of the peptide array (microarray) was performed as published elsewhere (Barouch et al., (2013) J Infect Dis 207: 248-256). Incubation of the microarrays was performed in an automated Hybridization Station and all steps were carried out at 30° C. The serum was diluted 1:200 in diluent buffer SuperBlock T20 (TBS, Pierce). The microarrays were blocked for 1 hour, incubated with the diluted serum for 2 hours and then incubated with Cy5-conjugated AffiniPure Mouse Anti-Human IgG secondary antibody (Jackson Immuno) for 45 minutes, with washing steps with 1×TBS+0.1% Tween20 in between. After the final wash steps microarrays were dried with nitrogen and the signal was read out with a Microarray Scanner. The signal intensity was displayed by relative fluorescence units and heatmaps of signal intensities for individual proteins were then evaluated (see FIGS. 7-15).

### Results:

A p-value was calculated for each peptide for the null hypothesis that the distribution of signal intensities is equal between patient groups:

a) CFS-Patients vs. healthy volunteers (NORM)
b) CFS-Patients vs. Depression Patients (DEP)

It was found that 114 peptide fragments fulfill the condition of p<0.01 for the comparison of signals from the CFS group to all other groups. These specific protein fragments are disclosed as SEQ ID NOs.: 22-135 in Table 1. In the table below, the number of peptides from each protein that are distinct between CFS patients and the other patient groups is shown:

TABLE 2

| Protein | BZLF1 | EBNA1 | EBNA3 | EBNA4 | EBNA6 | LMP1 | VP26 |
|---|---|---|---|---|---|---|---|
| No. of | 8 | 7 | 27 | 23 | 40 | 5 | 4 |
| peptides | | | | | | | |

From the above Table 2, it is apparent that the most differences were seen in the immune response towards peptides from EBNA3, EBNA4 and EBNA6. From this, it was concluded that these three peptides are the most suitable markers for the diagnosis of CFS, and that these also allow for the differentiation between CFS patients and depression patients.

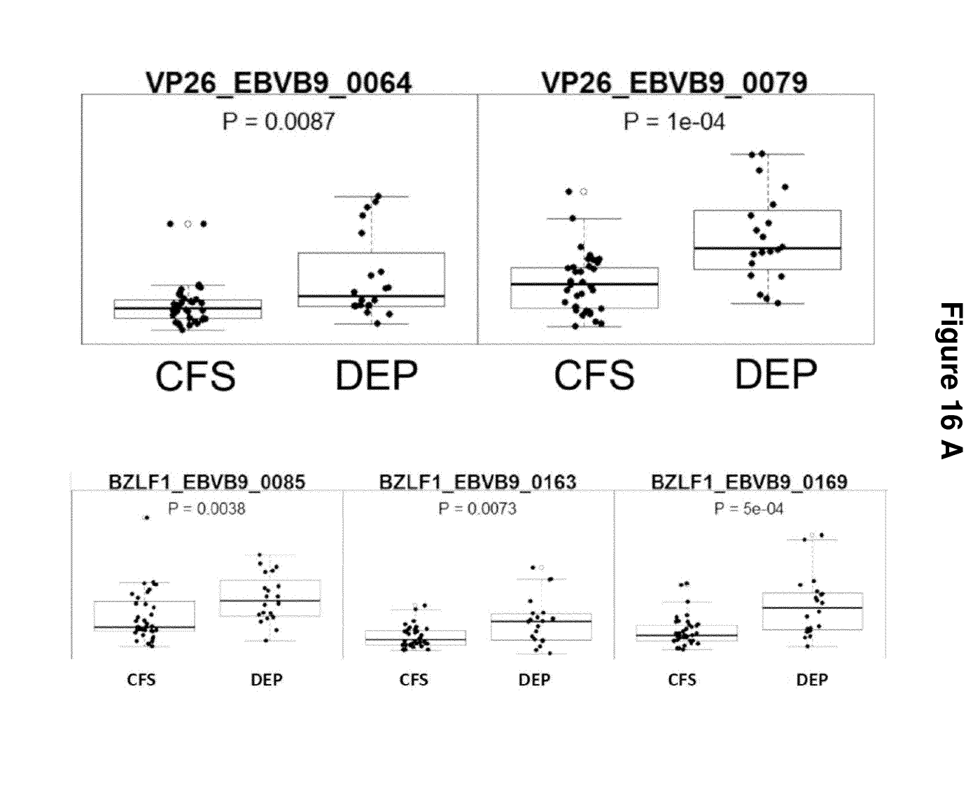

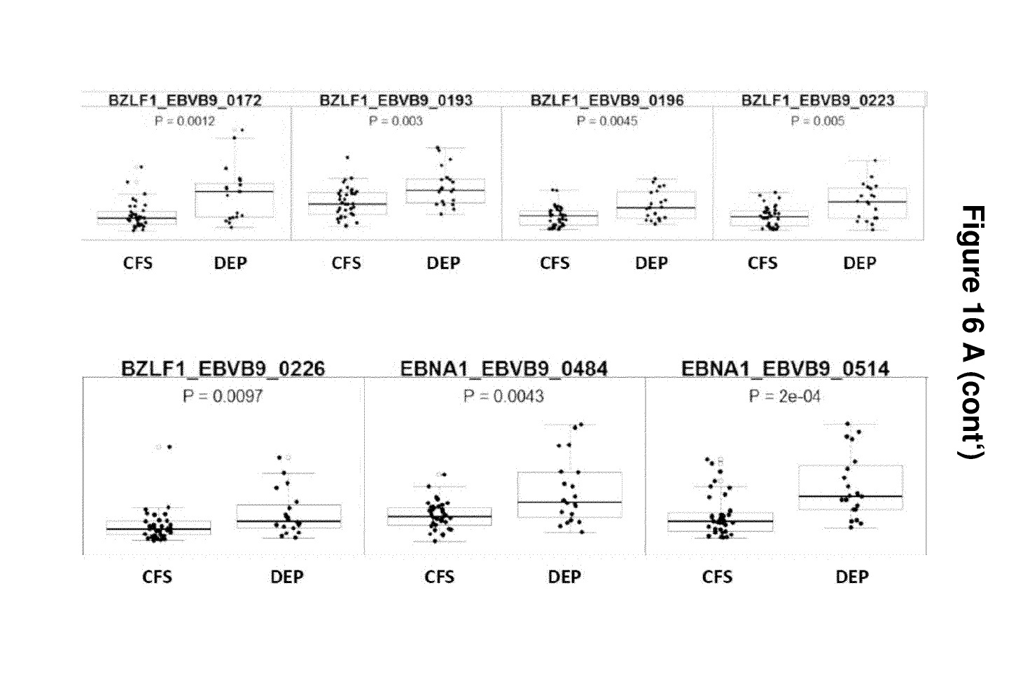

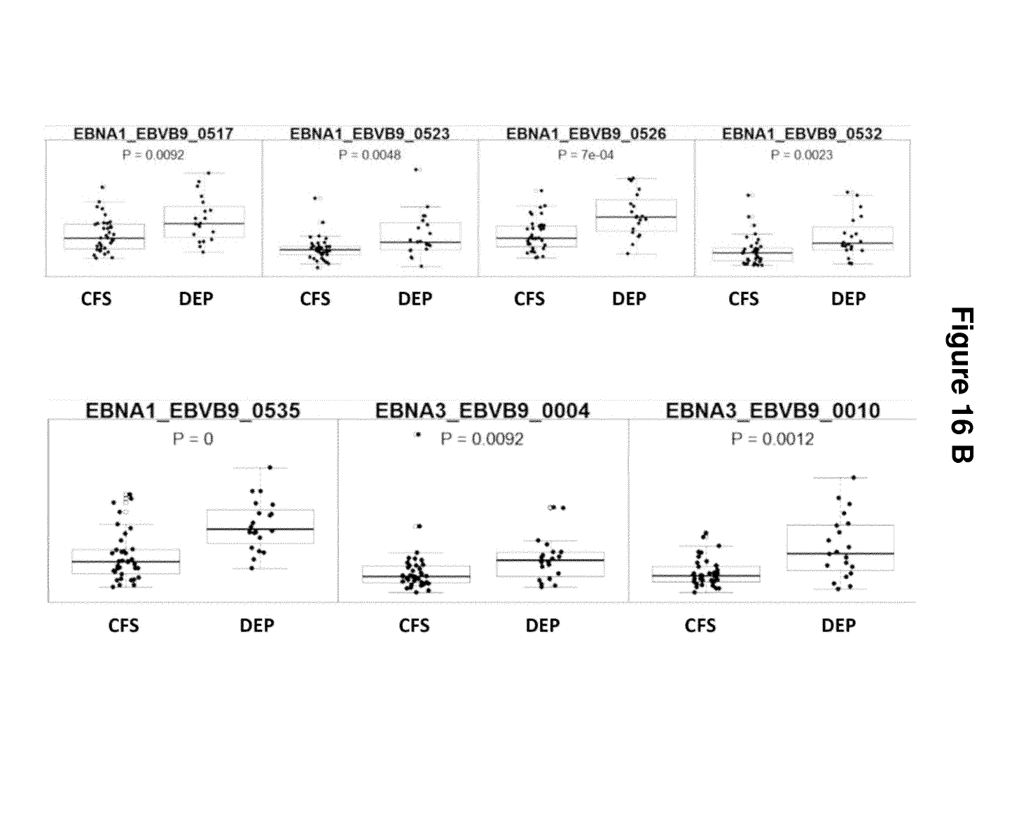

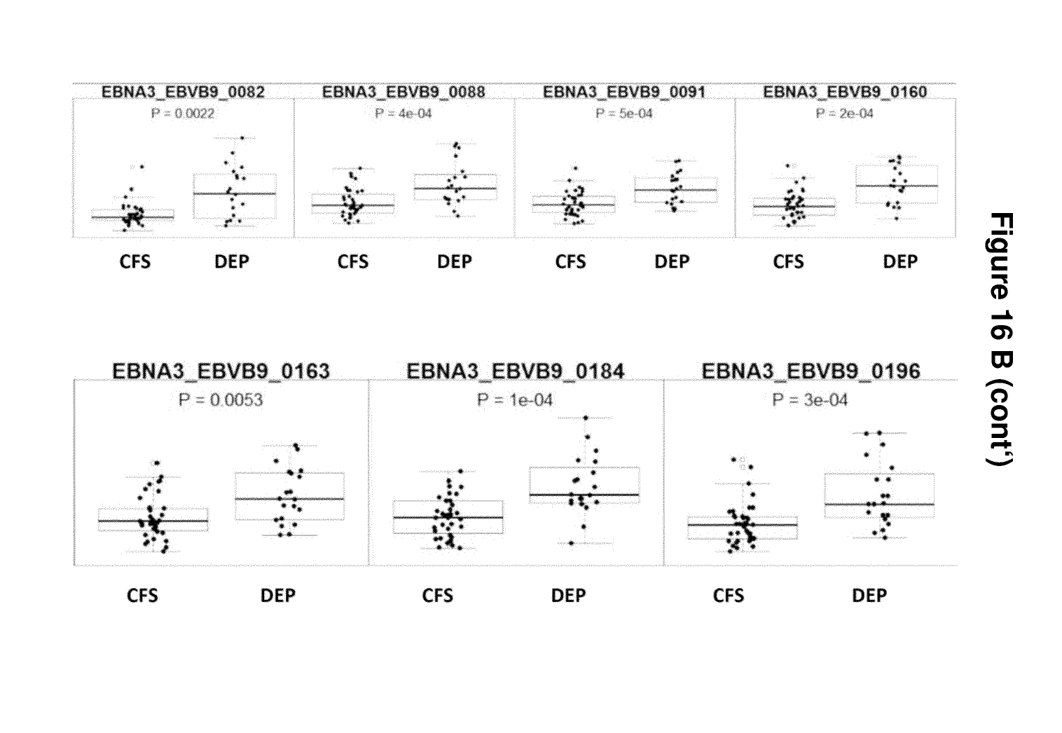

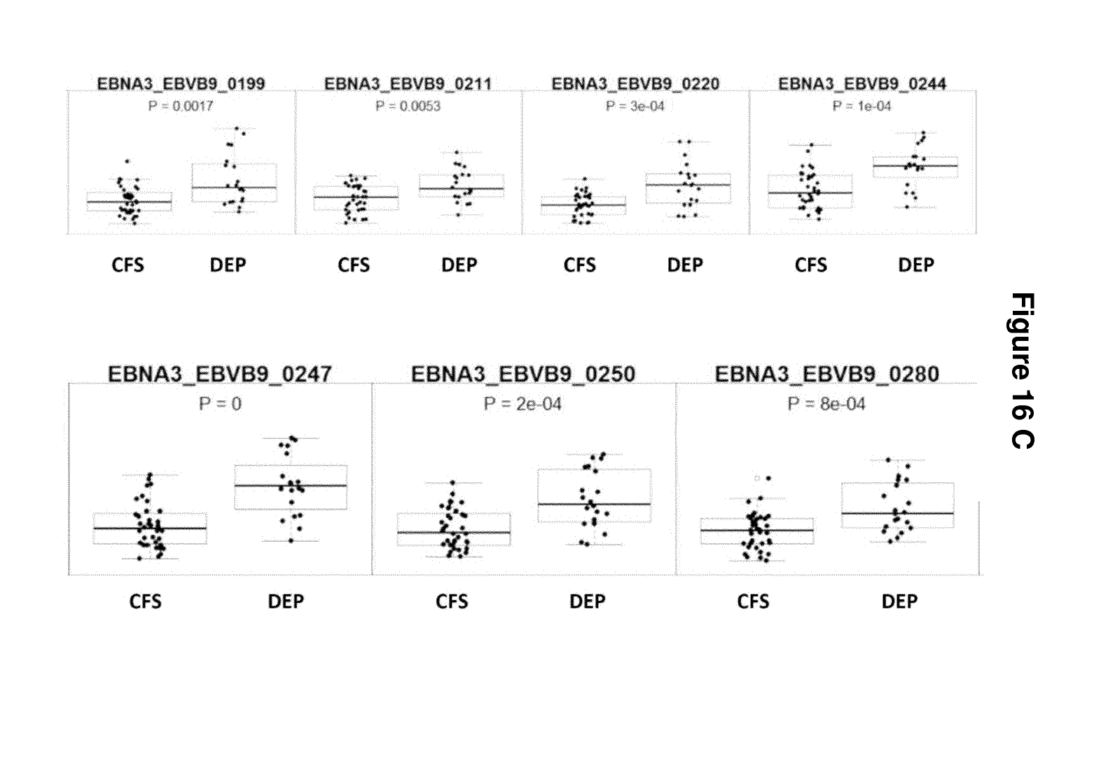

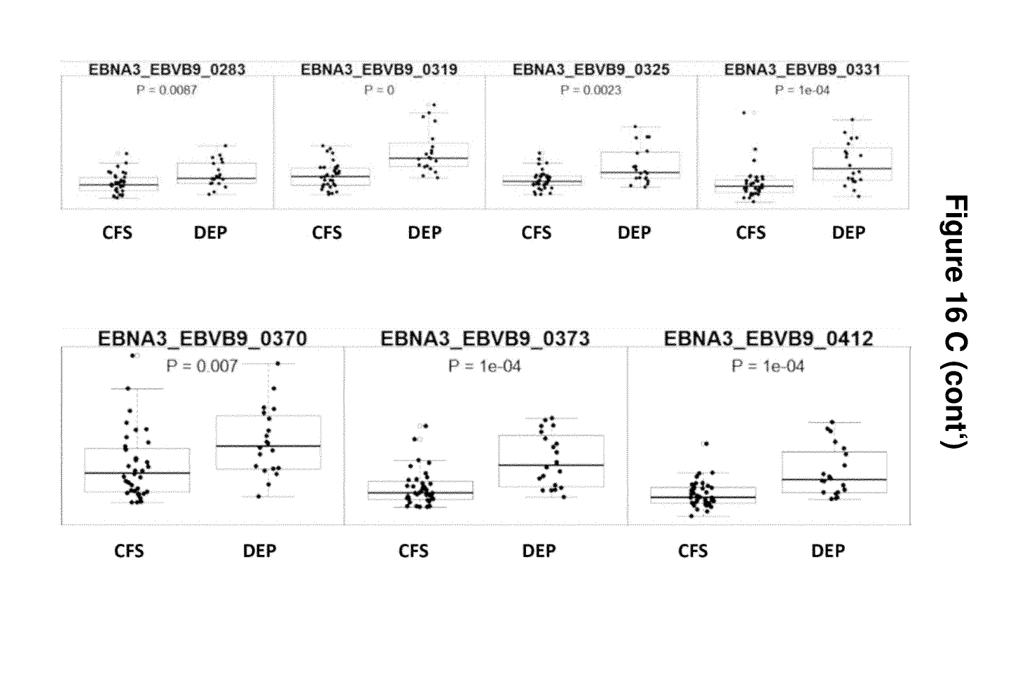

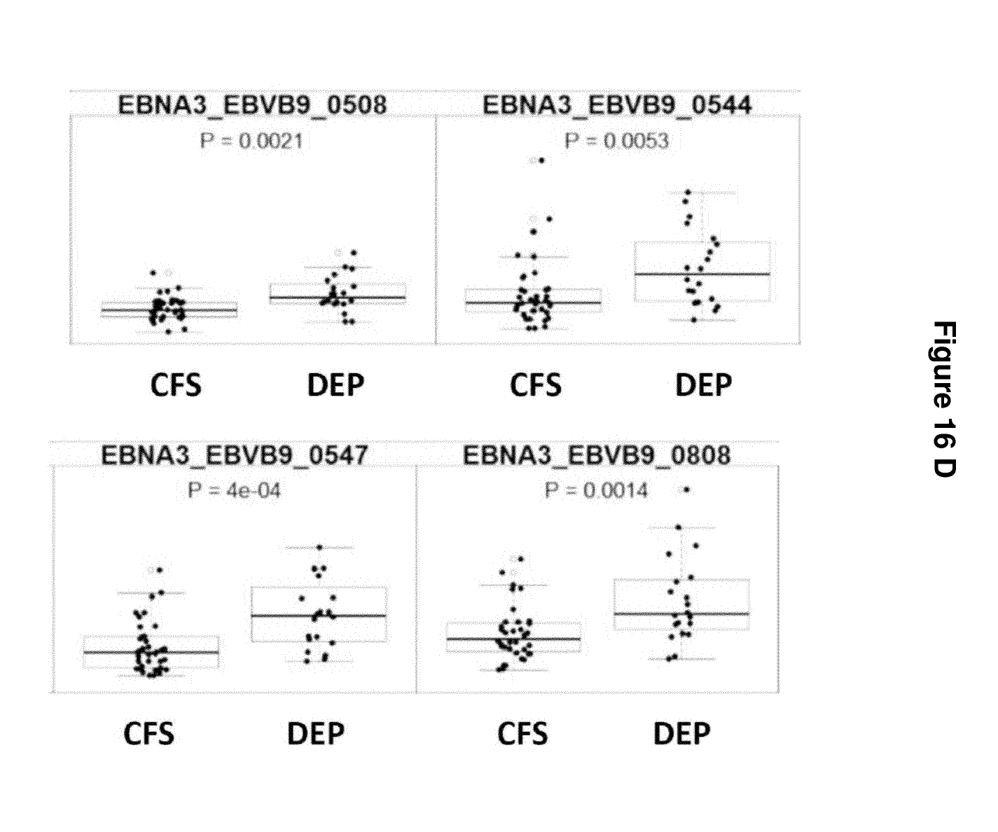

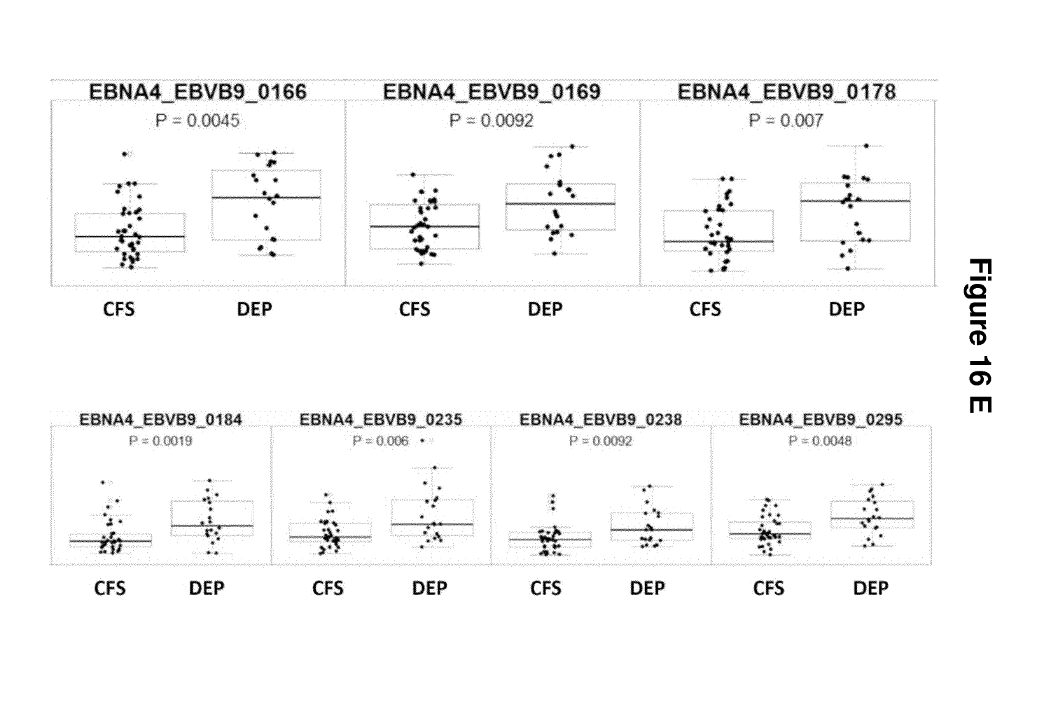

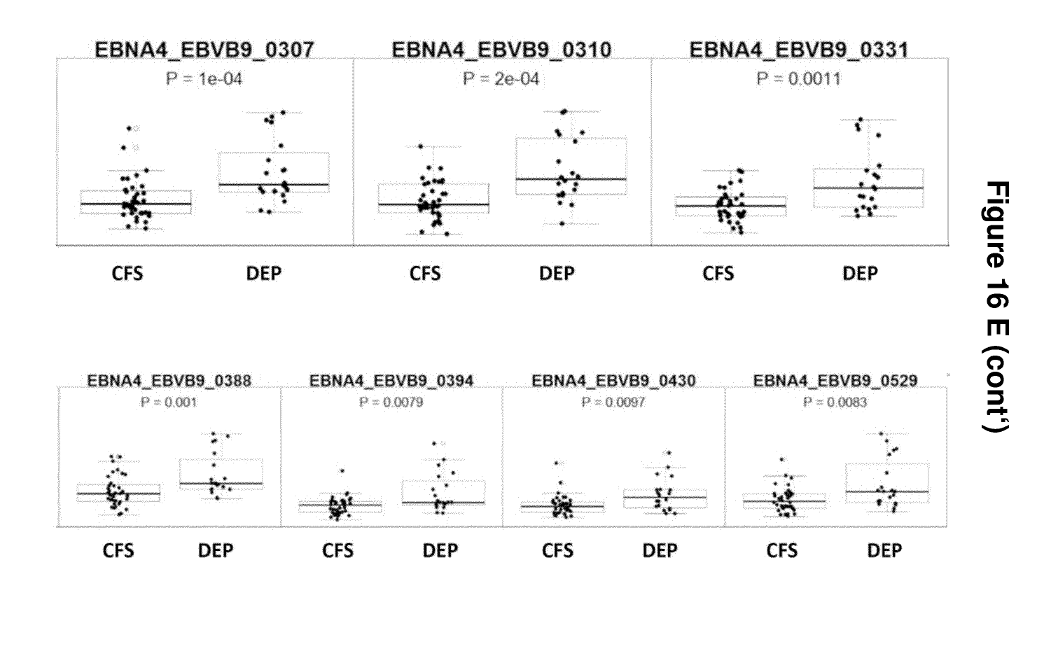

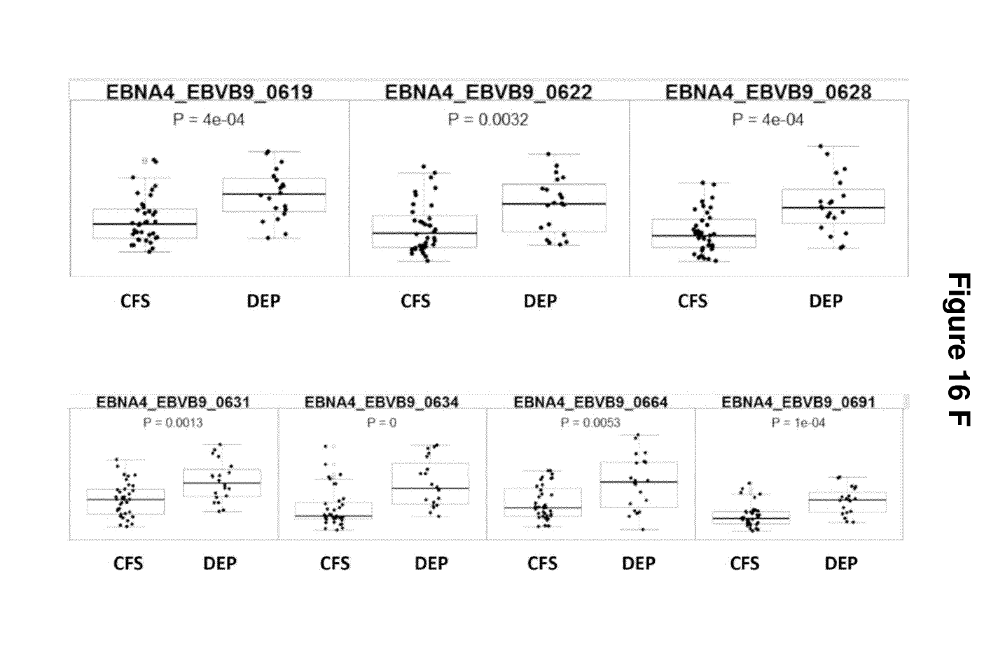

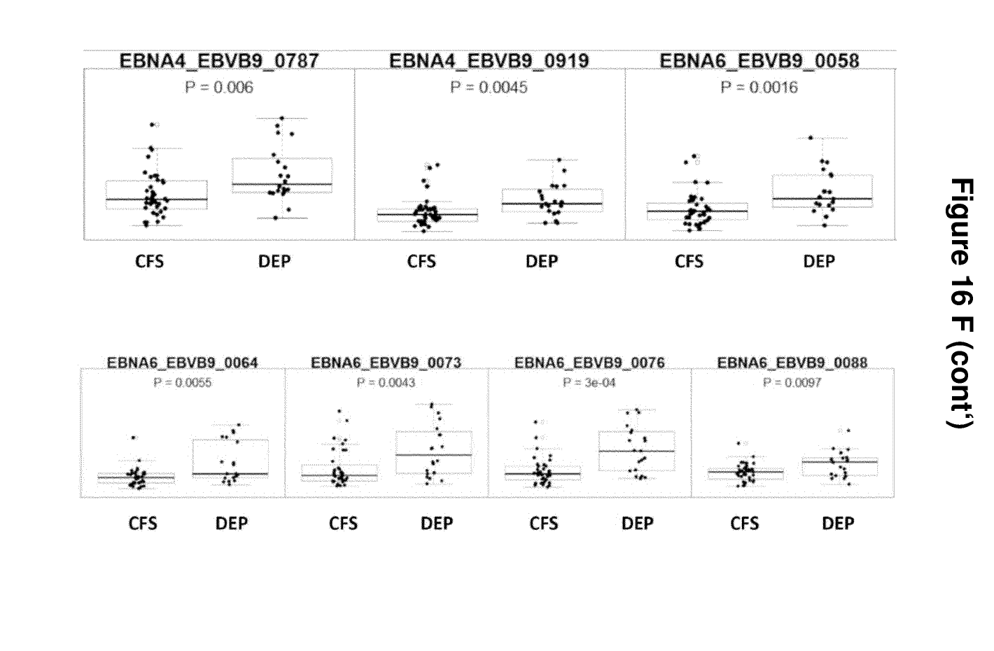

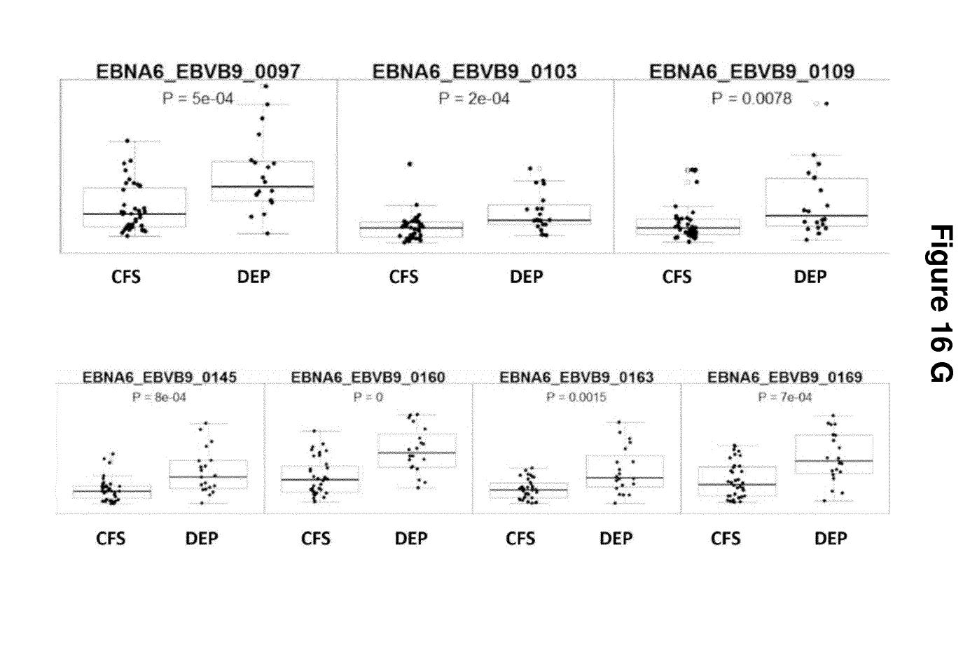

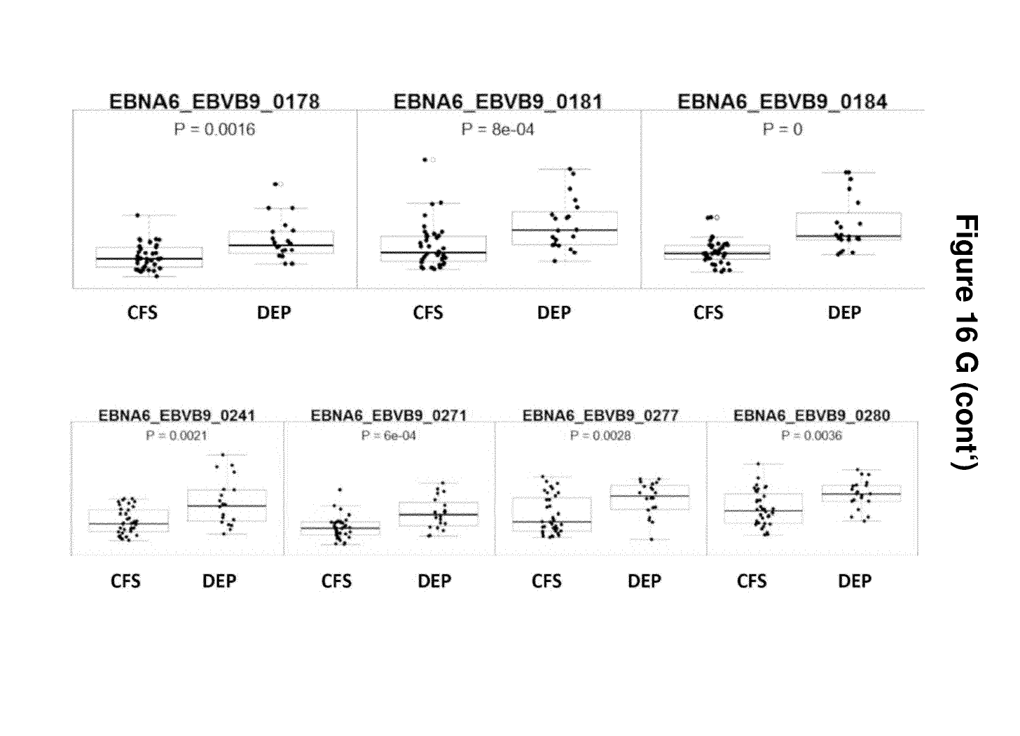

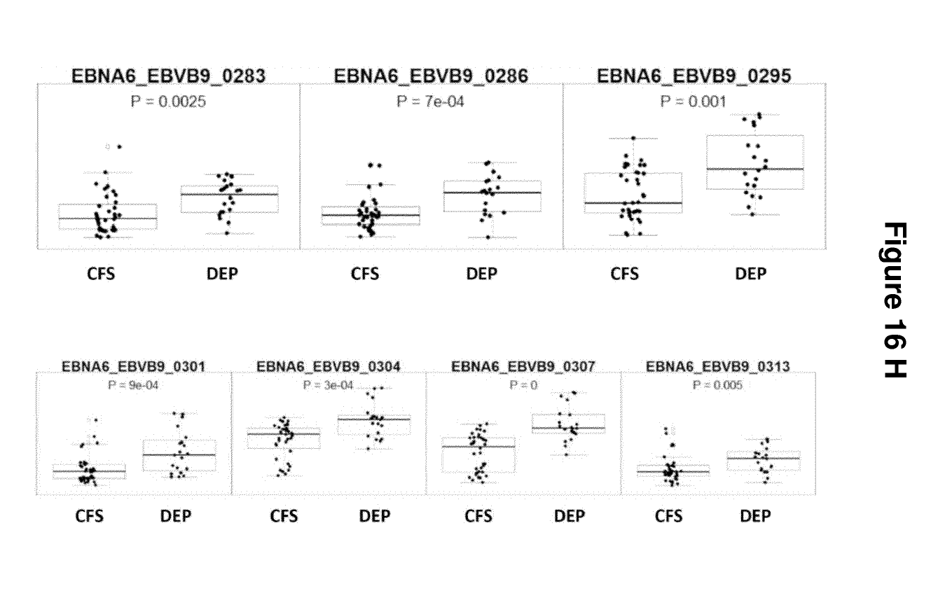

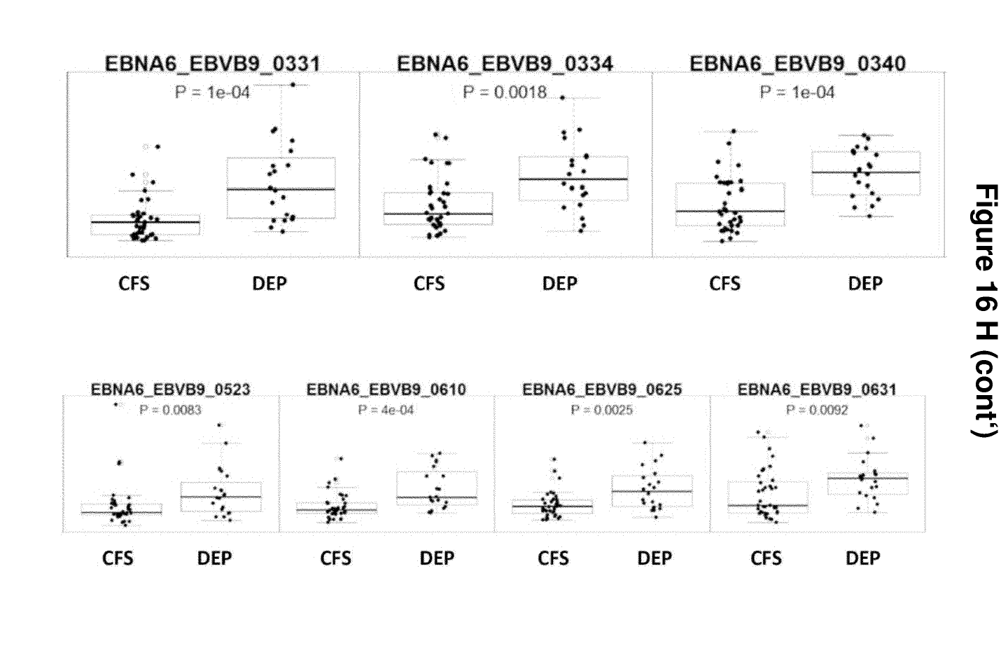

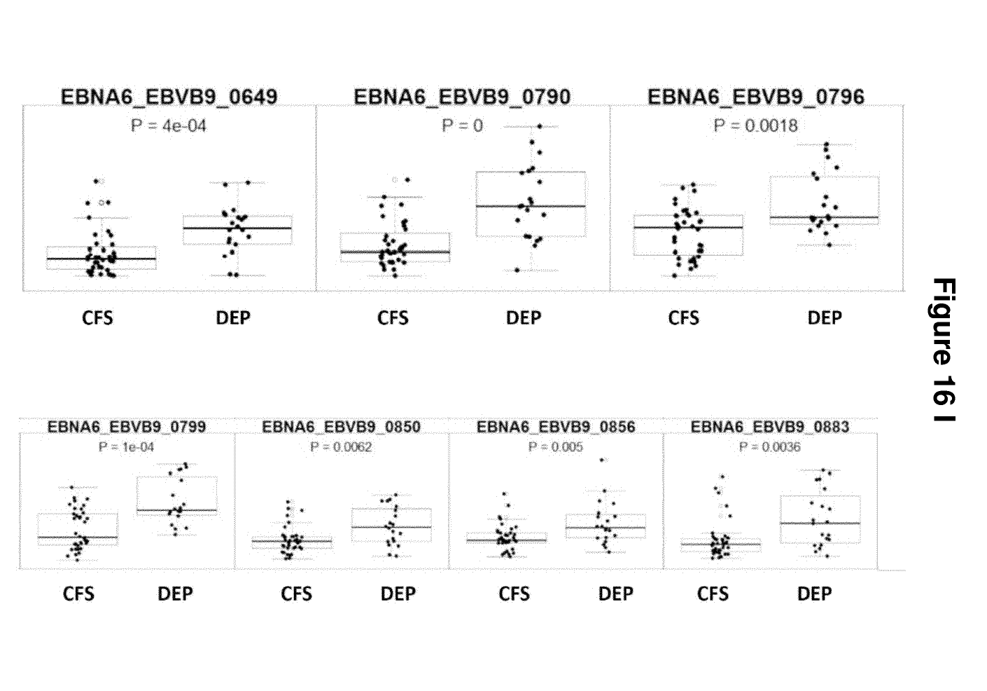

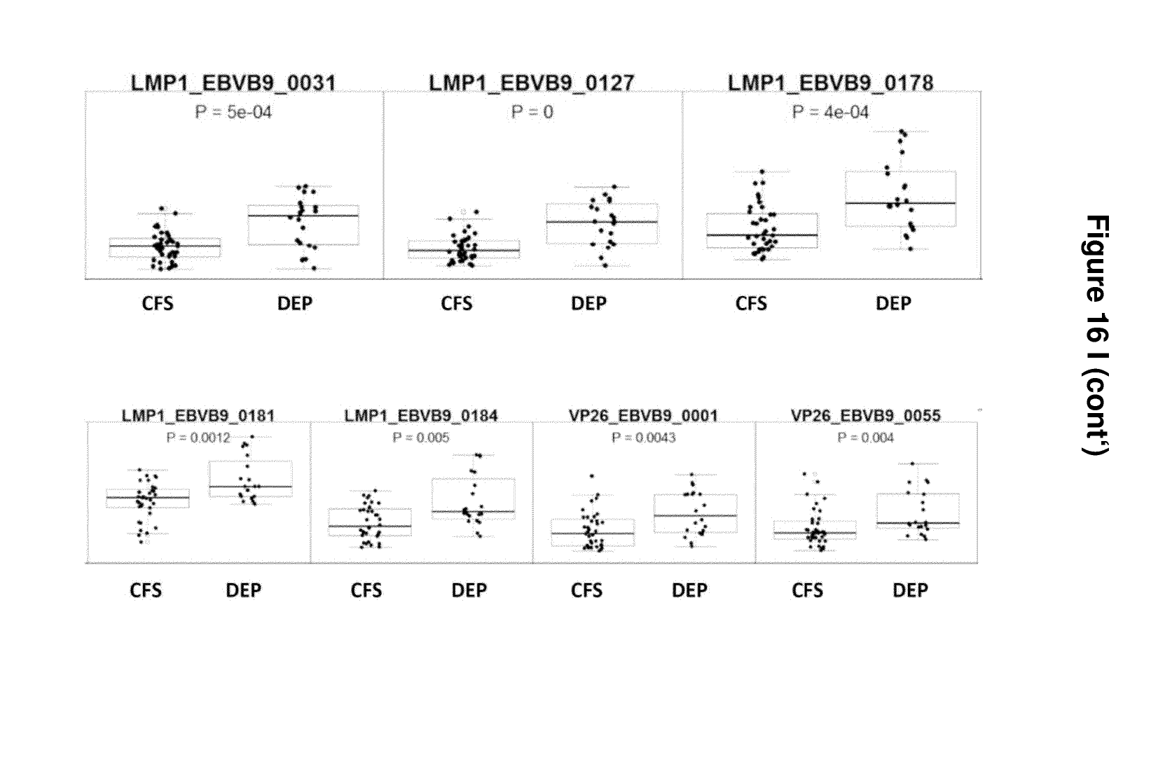

For the evaluation of the foregoing results, Boxplots of signal intensities for individual peptides fragments with a p-value<0.01: CFS patients vs. Depression patients are shown in FIG. 16A to 16I.

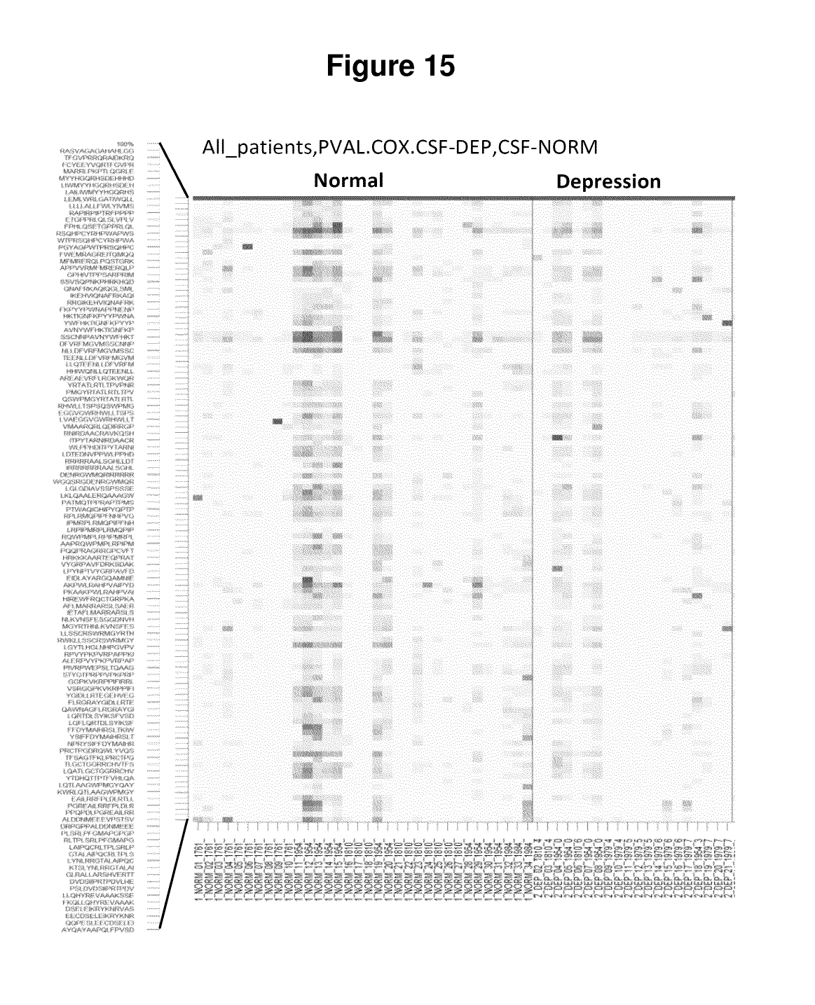

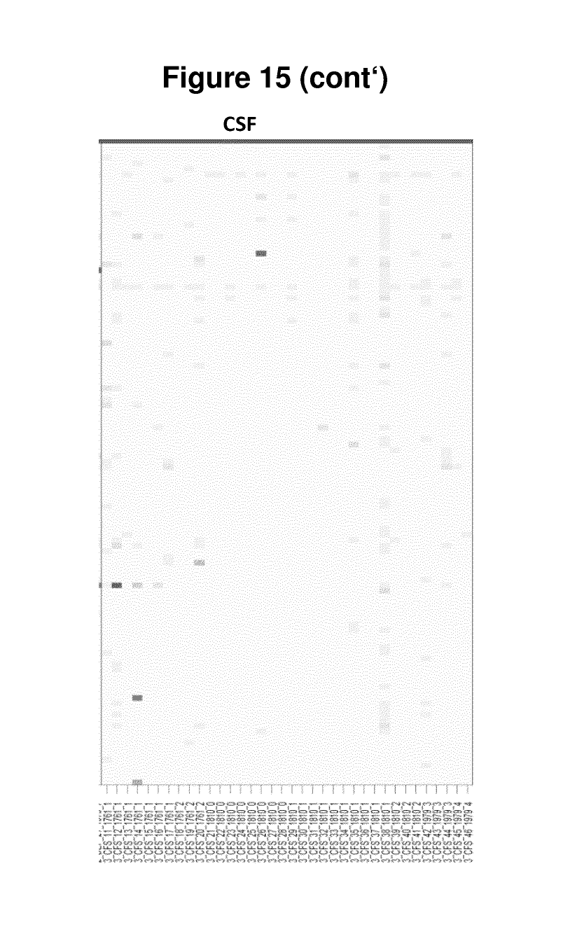

In FIG. 15 a p-value was calculated for each peptide for the null hypothesis that the distribution of signal intensities is equal between patient groups:
a) CFS-Patients vs. healthy volunteers (NORM)
b) CFS-Patients vs. Depression Patients (DEP).

The invention illustratively described herein may suitably be practiced in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by exemplary embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

1. An in vitro method for diagnosing Chronic Fatigue Syndrome, the method comprising determining the presence, absence or the amount of at least one marker characteristic of Epstein-Barr virus (EBV) infection in a sample obtained from the body of an individual, wherein the marker is selected from the group consisting of EBNA1, EBNA3, EBNA4, EBNA6, BZLF1, LMP1 and VP26, and wherein the presence of Chronic Fatigue Syndrome can be concluded from the presence, absence or the amount of the marker in the sample.

2. The method of claim 1, wherein at least one, two or all of the markers EBNA1, EBNA3, EBNA4 and EBNA6 is absent in the sample and the absence indicates the presence of Chronic Fatigue Syndrome.

3. The method of claim 1, wherein at least one, two or all of the markers BZLF1 and VP26 is present in the sample and the presence indicates the presence of Chronic Fatigue Syndrome.

4. The method of claim 1, wherein the presence, absence or amount of immune response to at least one further marker, which is characteristic of Epstein-Barr virus infection, is determined.

5. The method of claim 1, wherein a distinction between Chronic Fatigue Syndrome and a physical disorder can be made by determining the presence, absence or amount of at least one of the markers EBNA3, EBNA4, and EBNA6, wherein the physical disorder can be depression or burnout syndrome.

6. The method of claim 1, wherein the presence, absence of the amount of the marker can be determined directly or indirectly by an assay suitable to detect the presence, absence or the amount of the marker.

7. The method according to claim 6, wherein the assay can determine the presence, absence or amount of each marker directly by measuring the presence, absence or amount of the corresponding protein or a fragment thereof or the corresponding nucleotide encoding the protein, or wherein the presence, absence or the amount of the marker can be determined indirectly by measuring the presence, absence of amount of an immune response to the corresponding protein or a fragment thereof.

8. The method according to claim 7, wherein the immune response is determined by an immunoassay.

9. The method according to claim 8, wherein the immunoassay comprises at least one protein fragment of 4 to 70 amino acids in length from the specified regions of the following sequences: amino acid 80-105 of SEQ ID NO.: 1; amino acid 158-245 of SEQ ID NO.: 1; amino acid 80-105 of SEQ ID NO.: 2; amino acid 158-245 of SEQ ID NO.: 2; amino acid 80-105 of SEQ ID NO.: 3; amino acid 158-245 of SEQ ID NO.: 3; amino acid 479-555 of SEQ ID NO.: 4; amino acid 479-555 of SEQ ID NO.: 5; amino acid 394-414 of SEQ ID NO: 5; amino acid 479-555 of SEQ ID NO.: 6; amino acid 1-30 of SEQ ID NO.: 7; amino acid 76-110 of SEQ ID NO.: 7; amino acid 154-431 of SEQ ID NO.: 7; amino acid 489-548 of SEQ ID NO.: 7; amino acid 784-828 of SEQ ID NO.: 7; amino acid 1-30 of SEQ ID NO.: 8; amino acid 77-111 of SEQ ID NO.: 8; amino acid 155-432 of SEQ ID NO.: 8; amino acid 503-567 of SEQ ID NO.: 8; amino acid 803-828 of SEQ ID NO.: 8; amino acid 1-30 of SEQ ID NO.: 9; amino acid 77-111 of SEQ ID NO.: 9; amino acid 155-432 of SEQ ID NO.: 9; amino acid 503-567 of SEQ ID NO.: 9; amino acid 794-819 of SEQ ID NO.: 9; amino acid 161-204 of SEQ ID NO.: 10; amino acid 230-259 of SEQ ID NO.: 10; amino acid 290-351 of SEQ ID NO.: 10; amino acid 383-450 of SEQ ID NO.: 10; amino acid 524-549 of SEQ ID NO.: 10; amino acid 614-719 of SEQ ID NO.: 10; amino acid 790-815 of SEQ ID NO.: 10; amino acid 922-946 of SEQ ID NO.: 10; amino acid 161-204 of SEQ ID NO.: 11; amino acid 230-259 of SEQ ID NO.: 11; amino acid 290-351 of SEQ ID NO.: 11; amino acid 383-450 of SEQ ID NO.: 11; amino acid 524-549 of SEQ ID NO.: 11; amino acid 614-711 of SEQ ID NO.: 11; amino acid 782-807 of SEQ ID NO.: 11; amino acid 914-938 of SEQ ID NO.: 11; amino acid 161-204 of SEQ ID NO.: 12; amino acid 230-259 of SEQ ID NO.: 12; amino acid 290-351 of SEQ ID NO.: 12; amino acid 383-450 of SEQ ID NO.: 12; amino acid 524-549 of SEQ ID NO.: 12; amino acid 614-711 of SEQ ID NO.: 12; amino acid 782-807 of SEQ ID NO.: 12; amino acid 914-938 of SEQ ID NO.: 12; amino acid 53-204 of SEQ ID NO.: 13; amino acid 236-306 of SEQ ID NO.: 13; amino acid 519-575 of SEQ ID NO.: 13; amino acid 632-696 of SEQ ID NO.: 13; amino acid 855-980 of SEQ ID NO.: 13; amino acid 53-204 of SEQ ID NO.: 14; amino acid 236-306 of SEQ ID NO.: 14; amino acid 518-543 of SEQ ID NO.: 14; amino acid 605-669 of SEQ ID NO.: 14; amino acid 785-903 of SEQ ID NO.: 14; amino acids 741-779 of SEQ ID NO: 14, amino acid 53-204 of SEQ ID NO.: 15; amino acid 236-306 of SEQ ID NO.: 15; amino acid 518-543 of SEQ ID NO.: 15; amino acid 570-634 of SEQ ID NO.: 15; amino acid 802-920 of SEQ ID NO.: 15; amino acid 26-51 of SEQ ID NO.: 16; amino acid 122-147 of SEQ ID NO.: 16; amino acid 173-204 of SEQ ID NO.: 16; amino acid 26-51 of SEQ ID NO.: 17; amino acid 122-147 of SEQ ID NO.: 17; amino acid 173-204 of SEQ ID NO.: 17; amino acid 26-51 of SEQ ID NO.: 18; amino acid 122-147 of SEQ ID NO.: 18; amino acid 173-204 of SEQ ID NO.: 18; amino acid 26-51 of SEQ ID NO.: 19; amino acid 122-147 of SEQ ID NO.: 19; amino acid 173-204 of SEQ ID NO.: 19; amino acid 26-51 of SEQ ID NO.: 20; amino acid 122-147 of SEQ ID NO.: 20; amino acid 173-204 of SEQ ID NO.: 20; amino acid 1-21 of SEQ ID NO.: 21; or amino acid 50-99 of SEQ ID NO.: 21, or any one of SEQ ID NOs.:22-135, of SEQ ID NO: 149-151 or SEQ ID NOs: 153-155).

10. The method according to claim 9, wherein the protein fragments are immobilized on a solid phase and brought into contact with the sample obtained from the body of the individual, to determine the immune response (antibody response) of the individual to at least one marker which are selected from the group consisting of EBNA1, EBNA3, EBNA4, EBNA6, BZLF1, LMP1 and VP26.

11. The method of claim 1, wherein the method further comprises determining the number of memory B cells of the individual, wherein the total number of memory B cells and the number of EBV-specific memory B cells is determined, wherein a reduction in the number of EBV-specific memory B cells relative to the number of EBV specific memory B-cells from a EBV seropositive control group of healthy individuals indicates the presence of Chronic Fatigue Syndrome.

12. The method of claim 1, wherein the method further comprises determining the response of TNF-alpha or interferon gamma-producing memory T cells to EBV protein or peptides, wherein a reduction of TNF-alpha or interferon gamma-producing memory T cells specific to EBV relative to the number of TNF-alpha or interferon gamma-producing memory T cells of an EBV seropositive control group of healthy individuals indicates the presence of Chronic Fatigue Syndrome.

13. The method of claim 1, wherein a device is used that comprises a solid phase having immobilized thereon a protein comprising or consisting of at least one marker or protein fragment thereof, wherein the marker is selected from the group consisting of EBNA1, EBNA3, EBNA4, EBNA6, BZLF1, LMP1 and VP26.

14. (canceled)

15. The method of claim 1, wherein at least one of the markers is EBNA3, EBNA4 or EBNA6 or a fragment thereof.

16. A device for the diagnosis of Chronic Fatigue Syndrome, wherein the device comprises a solid phase having immobilized thereon a protein comprising or consisting of at least one marker or protein fragment thereof, wherein the marker is selected from the group consisting of EBNA1, EBNA3, EBNA4, EBNA6, BZLF1, LMP1 and VP26.
